(19) United States
(12) Patent Application Publication
(10) Pub. No.: US 2013/0034495 A1
(43) Pub. Date: Feb. 7, 2013

(54) ISOTOPOLOGUES OF 4-[9-(TETRAHYDRO-FURAN-3-YL)-8-(2,4,6-TRIFLUORO-PHENYLAMINO)-9H-PURIN-2-YLAMINO]-CYCLOHEXAN-1-OL

(71) Applicants: Marie Georges Beauchamps, Flanders, NJ (US); Louise Michelle Cameron, Nazareth, PA (US); Mohit Atul Kothare, Bridgewater, NJ (US); Manohar T. Saindane, Monmouth Junction, NJ (US)

(72) Inventors: Marie Georges Beauchamps, Flanders, NJ (US); Louise Michelle Cameron, Nazareth, PA (US); Mohit Atul Kothare, Bridgewater, NJ (US); Manohar T. Saindane, Monmouth Junction, NJ (US)

(21) Appl. No.: 13/514,319

(22) PCT Filed: Dec. 9, 2009
(86) PCT No.: PCT/US09/67313

§ 371 Date: Oct. 15, 2012

(51) Int. Cl. C07D 473/32; A61P 35/00; A61K 51/04; A61P 3/00; A61P 9/00; A61P 29/00; A61K 31/52; A61P 37/00

(52) U.S. Cl. 424/1.81; 544/277; 514/263.23

(57) ABSTRACT

Provided herein are isotopologues of Compound 1, which are enriched with isotopes such as, for example, deuterium. Pharmaceutical compositions comprising the isotope-enriched compounds, and methods of using such compounds are also provided.

### 1. FIELD

Provided herein are isotopologues of certain haloaryl substituted aminopurine compounds, compositions comprising the isotopologues, methods of making the isotopologues, and methods of their use for treatment or prevention of diseases and conditions including, but not limited to, inflammatory diseases, autoimmune diseases, and cancers.

### 2. BACKGROUND

The connection between abnormal protein phosphorylation and the cause or consequence of diseases has been known for over 20 years. Accordingly, protein kinases have become a very important group of drug targets. See Cohen, Nature, 1:309-315 (2002). Various protein kinase inhibitors have been used clinically in the treatment of a wide variety of diseases, such as cancer and chronic inflammatory diseases, including diabetes and stroke. See Cohen, Eur. J. Biochem., 268:5001-5010 (2001).

Because protein kinases regulate nearly every cellular process, including metabolism, cell proliferation, cell differentiation, and cell survival, they are attractive targets for therapeutic intervention for various disease states. For example, cell-cycle control and angiogenesis, in which protein kinases play a pivotal role are cellular processes associated with numerous disease conditions such as but not limited to cancer, inflammatory diseases, abnormal angiogenesis and diseases related thereto, atherosclerosis, macular degeneration, diabetes, obesity, and pain.

Protein kinases have become attractive targets for the treatment of cancers. Fabbro et al., Pharmacology & Therapeutics 93:79-98 (2002). It has been proposed that the involvement of protein kinases in the development of human malignancies may occur by: (1) genomic rearrangements (e.g., BCR-ABL in chronic myelogenous leukemia), (2) mutations leading to constitutively active kinase activity, such as acute myelogenous leukemia and gastrointestinal tumors, (3) deregulation of kinase activity by activation of oncogenes or loss of tumor suppressor functions, such as in cancers with oncogenic RAS, (4) deregulation of kinase activity by over-expression, as in the case of EGFR and (5) ectopic expression of growth factors that can contribute to the development and maintenance of the neoplastic phenotype. Fabbro et al., Pharmacology & Therapeutics 93:79-98 (2002).

The elucidation of the intricacy of protein kinase pathways and the complexity of the relationship and interaction among and between the various protein kinases and kinase pathways highlights the importance of developing pharmaceutical agents capable of acting as protein kinase modulators, regulators or inhibitors that have beneficial activity on kinases or kinase pathways. Accordingly, there remains a need for new kinase modulators.

The compound chemically named 4-[9-(tetrahydro-furan-3-yl)-8-(2,4,6-trifluoro-phenylamino)-9H-purin-2-ylamino]-cyclohexan-1-ol was disclosed in U.S. patent application Ser. No. 11/332,617, filed Jan. 12, 2006, International Pub. No. WO 2006/076595, U.S. patent application Ser. No. 11/977,759, filed Oct. 26, 2007, International Pub. No. WO 2008/057252, the entireties of each of which is incorporated herein by reference, and was shown to have utility for, inter alia, treatment of cancer, cardiovascular disease, renal disease, autoimmune conditions, inflammatory conditions, macular degeneration, ischemia-reperfusion injury, pain and related syndromes, disease-related wasting, asbestos-related conditions, pulmonary hypertension, central nervous system (CNS) injury/damage or conditions treatable or preventable by inhibition of a kinase pathway.

Citation or identification of any reference in Section 2 of this application is not to be construed as an admission that the reference is prior art to the present application.

### 3. SUMMARY

Embodiments provided herein encompass particular isotopologues of 4-[9-(tetrahydro-furan-3-yl)-8-(2,4,6-trifluoro-phenylamino)-9H-purin-2-ylamino]-cyclohexan-1-ol (“Compound 1”) or a pharmaceutically acceptable salt or solvate thereof. Compound 1 has the following structure:

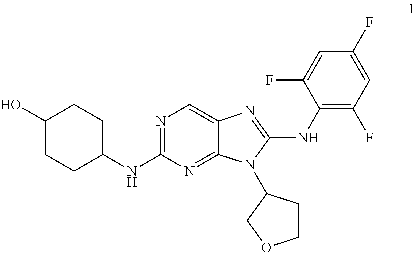 [SMILES: OC1CCC(Nc2ncc3nc(Nc4c(F)cc(F)cc4F)n(C4CCOC4)c3n2)CC1]

Compound 1 encompasses the following molecules, and mixtures thereof:

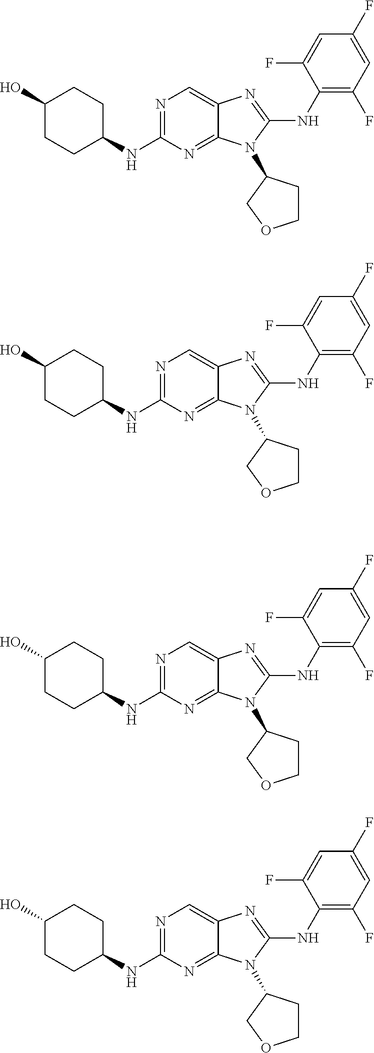 [SMILES: O[C@H]1CC[C@@H](Nc2ncc3nc(Nc4c(F)cc(F)cc4F)n([C@H]4CCOC4)c3n2)CC1 ; O[C@H]1CC[C@@H](Nc2ncc3nc(Nc4c(F)cc(F)cc4F)n([C@@H]4CCOC4)c3n2)CC1 ; O[C@H]1CC[C@H](Nc2ncc3nc(Nc4c(F)cc(F)cc4F)n([C@H]4CCOC4)c3n2)CC1 ; O[C@H]1CC[C@H](Nc2ncc3nc(Nc4c(F)cc(F)cc4F)n([C@@H]4CCOC4)c3n2)CC1]

In one embodiment, the isotopologue is an isotopologue of

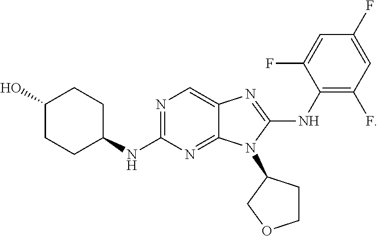 [SMILES: O[C@H]1CC[C@H](Nc2ncc3nc(Nc4c(F)cc(F)cc4F)n([C@H]4CCOC4)c3n2)CC1]

Certain embodiments encompass mixtures of isotopologues of Compound 1. Certain embodiments encompass methods of synthesizing, isolating, or characterizing the isotopologues of Compound 1. In certain embodiments, the isotopologues of Compound 1 are deuterium, carbon-13, nitrogen-15, or oxygen-18 enriched, or combinations thereof.

In certain embodiments, provided herein are pharmaceutical compositions and single unit dosage forms comprising one or more isotopologues of Compound 1. Certain embodiments provide methods for the treatment or prevention of particular diseases or disorders, which comprise administering to a patient a therapeutically or prophylactically effective amount of an isotopologue of Compound 1. In some embodiments, the isotopologues of Compound 1 are used in combination with one or more other therapeutic agents, as described herein.

### 4. DETAILED DESCRIPTION

The descriptions of the terminology provided below apply to the terms as used herein, unless otherwise specified.

As used herein “Compound 1” includes pharmaceutically acceptable salts, prodrugs, and solvates (e.g., hydrates) thereof.

The term “isotopic composition” refers to the amount of each isotope present for a given atom, and “natural isotopic composition” refers to the naturally occurring isotopic composition or abundance for a given atom. Atoms containing their natural isotopic composition may also be referred to herein as “non-enriched” atoms. Unless otherwise designated, the atoms of the compounds recited herein are meant to represent any stable isotope of that atom. For example, unless otherwise stated, when a position is designated specifically as “H” or “hydrogen,” the position is understood to have hydrogen at its natural isotopic composition.

The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. As used herein, an “isotopologue” is an isotopically enriched compound.

The term “isotopic enrichment” refers to the percentage of incorporation of an amount of a specific isotope at a given atom in a molecule in the place of that atom's natural isotopic composition. For example, deuterium enrichment of 1% at a given position means that 1% of the molecules in a given sample contain deuterium at the specified position. Because the naturally occurring distribution of deuterium is about 0.0156%, about 0.0156% of molecules in a sample synthesized using non-enriched starting materials will have deuterium at a given position.

The term “isotopic enrichment factor” refers to the ratio between the isotopic composition and the natural isotopic composition of a specified isotope.

With regard to the compounds provided herein, when a particular atomic position is designated as having deuterium or “D,” it is understood that the abundance of deuterium at that position is substantially greater than the natural abundance of deuterium, which is about 0.0156%. A position designated as having deuterium typically has a minimum isotopic enrichment factor of, in particular embodiments, at least 100 (1.56% deuterium incorporation), at least 500 (7.8% deuterium incorporation), at least 1000 (15.6% deuterium incorporation), at least 2000 (31.2% deuterium incorporation), at least 3000 (46.8% deuterium incorporation), at least 3500 (54.6% deuterium incorporation), at least 4000 (62.4% deuterium incorporation), at least 4500 (70.2% deuterium incorporation), at least 5000 (78% deuterium incorporation), at least 5500 (85.8% deuterium incorporation), at least 6000 (93.6% deuterium incorporation), at least 6089.7 (95% deuterium incorporation), at least 6217.9 (97% deuterium incorporation), at least 6346.2 (99% deuterium incorporation), or at least 6378.2 (99.5% deuterium incorporation) at each designated deuterium atom.

The isotopic enrichment and isotopic enrichment factor of the compounds provided herein can be determined using conventional analytical methods known to one of ordinary skill in the art, including mass spectrometry and nuclear magnetic resonance spectroscopy.

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the compounds described herein include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, maleic, phosphoric, sulfuric, and methanesulfonic acids. Examples of specific salts thus include hydrochloride and mesylate salts. Others are well-known in the art, see for example, Remington's Pharmaceutical Sciences, 18^{th} eds., Mack Publishing, Easton Pa. (1990) or Remington: The Science and Practice of Pharmacy, 19^{th} eds., Mack Publishing, Easton Pa. (1995).

As used herein and unless otherwise indicated, the term “solvate” means a compound as described herein, or a salt thereof, that further includes a stoichiometric or non-stoichiometric amount of a solvent bound by non-covalent intermolecular forces. In some embodiments, the solvate is a hydrate. As used herein and unless otherwise indicated, the term “hydrate” means a compound as described herein, or a salt thereof, that further includes a stoichiometric or non-stoichiometric amount of water bound by non-covalent intermolecular forces.

As used herein and unless otherwise indicated, the term “prodrug” means an derivative of a compound as described herein that can be hydrolyzed, oxidized, or otherwise reacted under biological conditions (in vitro or in vivo) to provide an active compound, particularly an isotopologue of Compound 1. Examples of prodrugs include, but are not limited to, derivatives and metabolites of a compound as described herein that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. In certain embodiments, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh).

As used herein and unless otherwise indicated, the term “stereoisomer” or “stereomerically pure” means one stereoisomer of a compound as described herein that is substantially free of other stereoisomers of that compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can occur as racemates, individual enantiomers or diastereomers, and mixtures thereof. All such isomeric forms are included within the embodiments disclosed herein, including mixtures thereof.

The use of stereomerically pure forms of the compounds as described herein, as well as the use of mixtures of those forms, are encompassed by the embodiments disclosed herein. For example, mixtures comprising equal or unequal amounts of the enantiomers of a particular compound as described herein may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, Ind., 1972).

It should also be noted the compounds as described herein can include E and Z isomers, or a mixture thereof, and cis and trans isomers or a mixture thereof. In certain embodiments, the compounds are isolated as either the E or Z isomer. In other embodiments, the compounds are a mixture of the E and Z isomers.

“Treating” as used herein, means alleviation, in whole or in part, of symptoms associated with a disorder or disease, or slowing, or halting of further progression or worsening of those symptoms, or prevention or prophylaxis of the disease or disorder in a subject at risk for developing the disease or disorder.

The term “effective amount” in connection with a compound as described herein can mean an amount capable of alleviating, in whole or in part, symptoms associated with a disorder or disease, or slowing or halting further progression or worsening of those symptoms, or preventing or providing prophylaxis for the disease or disorder in a subject having or at risk for developing a disease disclosed herein, such as cancer, cardiovascular disease, renal disease, autoimmune conditions, inflammatory conditions, macular degeneration, ischemia-reperfusion injury, pain and related syndromes, disease-related wasting, asbestos-related conditions, pulmonary hypertension, central nervous system (CNS) injury/damage or conditions treatable or preventable by inhibition of a kinase pathway. The effective amount of a compound as described herein, for example in a pharmaceutical composition, may be at a level that will exercise the desired effect; for example, about 0.005 mg/kg of a subject's body weight to about 10 mg/kg of a subject's body weight in unit dosage for both oral and parenteral administration.

### 4.1 Compounds

Provided herein are isotopically enriched compounds, including isotopically enriched Compound 1, synthetic intermediates thereof, and metabolites thereof.

Isotopic enrichment (e.g., deuteration) of pharmaceuticals to improve pharmacokinetics (“PK”), pharmacodynamics (“PD”), and toxicity profiles has been demonstrated previously with some classes of drugs. (See, e.g., Lijinsky et. al., Food Cosmet. Toxicol., 20: 393 (1982); Lijinsky et. al., J. Nat. Cancer Inst., 69: 1127 (1982); Mangold et. al., Mutation Res. 308: 33 (1994); Gordon et. al., Drug Metab. Dispos., 15: 589 (1987); Zello et. al., Metabolism, 43: 487 (1994); Gately et. al., J. Nucl. Med., 27: 388 (1986); Wade D, Chem. Biol. Interact. 117: 191 (1999)).

Without being limited by a particular theory, isotopic enrichment of a drug can be used, for example, to (1) reduce or eliminate unwanted metabolites, (2) increase the half-life of the parent drug, (3) decrease the number of doses needed to achieve a desired effect, (4) decrease the amount of a dose necessary to achieve a desired effect, (5) increase the formation of active metabolites, if any are formed, and/or (6) decrease the production of deleterious metabolites in specific tissues and/or create a more effective drug and/or a safer drug for combination therapy, whether the combination therapy is intentional or not.

Replacement of an atom for one of its isotopes may often result in a change in the reaction rate of a chemical reaction or an enzyme catalyzed reaction. This phenomenon is known as the Kinetic Isotope Effect (“KIE”). For example, if a C—H bond is broken during a rate-determining step in a chemical reaction (i.e. the step with the highest transition state energy), substitution of a deuterium for that hydrogen can cause a decrease in the reaction rate and the process may slow down. This phenomenon is known as the Deuterium Kinetic Isotope Effect (“DKIE”). (See, e.g, Foster et al., Adv. Drug Res., vol. 14, pp. 1-36 (1985); Kushner et al., Can. J. Physiol. Pharmacol., vol. 77, pp. 79-88 (1999)).

The magnitude of the DKIE can be expressed as the ratio between the rates of a given reaction in which a C—H bond is broken, and the same reaction where deuterium is substituted for hydrogen. The DKIE can range from about 1 (no isotope effect) to very large numbers, such as 50 or more, meaning that the reaction can be fifty, or more, times slower when deuterium is substituted for hydrogen. Without being limited by a particular theory, high DKIE values may be due in part to a phenomenon known as tunneling, which is a consequence of the uncertainty principle. Tunneling is ascribed to the small mass of a hydrogen atom, and occurs because transition states involving a proton can sometimes form in the absence of the required activation energy. Because deuterium has more mass than hydrogen, it statistically has a much lower probability of undergoing this phenomenon.

Tritium (“T”) is a radioactive isotope of hydrogen, used in research, fusion reactors, neutron generators and radiopharmaceuticals. Tritium is a hydrogen atom that has 2 neutrons in the nucleus and has an atomic weight close to 3. It occurs naturally in the environment in very low concentrations, most commonly found as T₂O. Tritium decays slowly (half-life=12.3 years) and emits a low energy beta particle that cannot penetrate the outer layer of human skin. Internal exposure is the main hazard associated with this isotope, yet it must be ingested in large amounts to pose a significant health risk. As compared with deuterium, a lesser amount of tritium must be consumed before it reaches a hazardous level. Substitution of tritium (“T”) for hydrogen results in yet a stronger bond than deuterium and gives numerically larger isotope effects. Similarly, substitution of isotopes for other elements, including, but not limited to, ¹³C or ¹⁴C for carbon, ³³S, ³⁴S, or ³⁶S for sulfur, ¹⁵N for nitrogen, and ¹⁷O or ¹⁸O for oxygen, may lead to a similar kinetic isotope effect.

The animal body expresses a variety of enzymes for the purpose of eliminating foreign substances, such as therapeutic agents, from its circulation system. Examples of such enzymes include the cytochrome P450 enzymes (“CYPs”), esterases, proteases, reductases, dehydrogenases, and monoamine oxidases, to react with and convert these foreign substances to more polar intermediates or metabolites for renal excretion. Some of the most common metabolic reactions of pharmaceutical compounds involve the oxidation of a carbon-hydrogen (C—H) bond to either a carbon-oxygen (C—O) or carbon-carbon (C—C) pi-bond. The resultant metabolites may be stable or unstable under physiological conditions, and can have substantially different pharmacokinetic, pharmacodynamic, and acute and long-term toxicity profiles relative to the parent compounds. For many drugs, such oxidations are rapid. These drugs therefore often require the administration of multiple or high daily doses.

Therefore, isotopic enrichment at certain positions of a compound provided herein may produce a detectable KIE that affects the pharmacokinetic, pharmacologic, and/or toxicological profiles of a compound provided herein in comparison with a similar compound having a natural isotopic composition. In one embodiment, the deuterium enrichment is performed on the site of C—H bond cleavage during metabolism.

In some embodiments, provided herein are deuterated analogues of Compound 1, wherein one or more atomic positions of Compound 1 is/are isotopically enriched with deuterium. Certain embodiments herein provide compounds of the following structure:

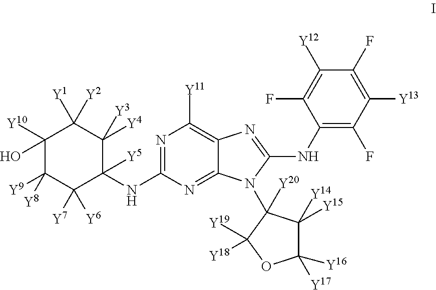

wherein one or more Y atoms (i.e., Y¹, Y¹, Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷, Y¹⁸, Y¹⁹, or Y²⁰) is/are hydrogen(s) isotopically enriched with deuterium, and any remaining Y atom(s) is/are non-enriched hydrogen atom(s). In particular embodiments, one, two, three, four, five, six, seven, or eight of the indicated Y atoms is/are isotopically enriched with deuterium, and any remaining Y atom(s) is/are non-enriched hydrogen(s). In one embodiment, all of Y¹, Y², Y², Y⁴, Y⁵, Y⁶, Y⁷, Y⁸, Y⁹, Y¹⁰, Y¹¹, Y², Y¹³, Y¹⁴, Y¹⁵, Y¹⁶, Y¹⁷, Y¹⁸, Y¹⁹, and Y²⁰ are isotopically enriched with deuterium.

In certain embodiments, one or more Y atoms on the cyclohexyl portion of Compound 1 are deuterium-enriched. For example, particular compounds provided herein include the following listed compounds, wherein the label “D” indicates a deuterium-enriched atomic position, i.e., a sample comprising the given compound has a deuterium enrichment at the indicated position(s) above the natural abundance of deuterium:

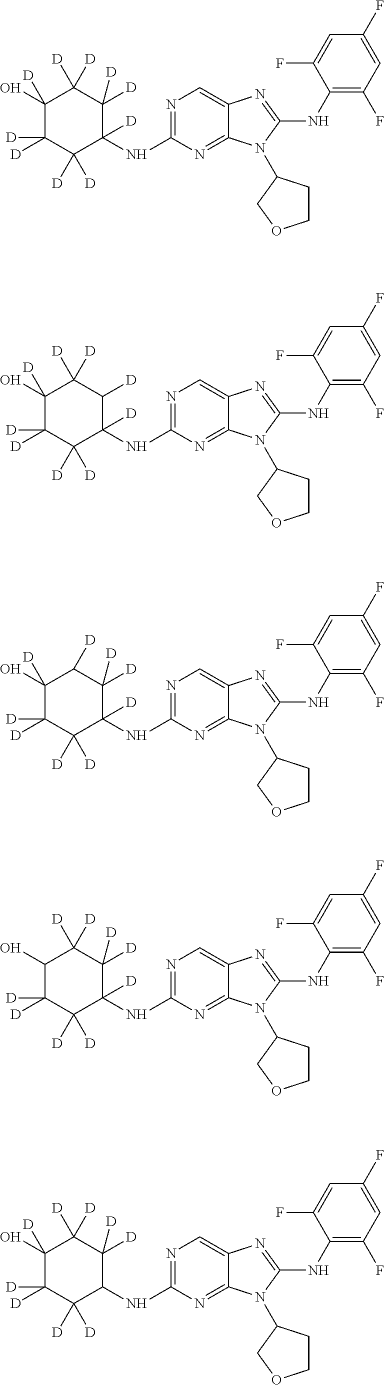

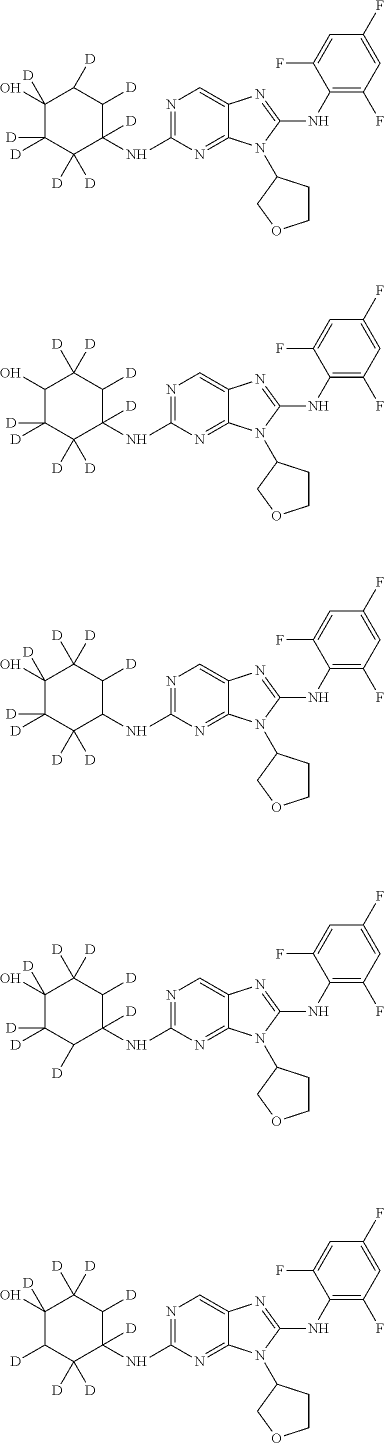

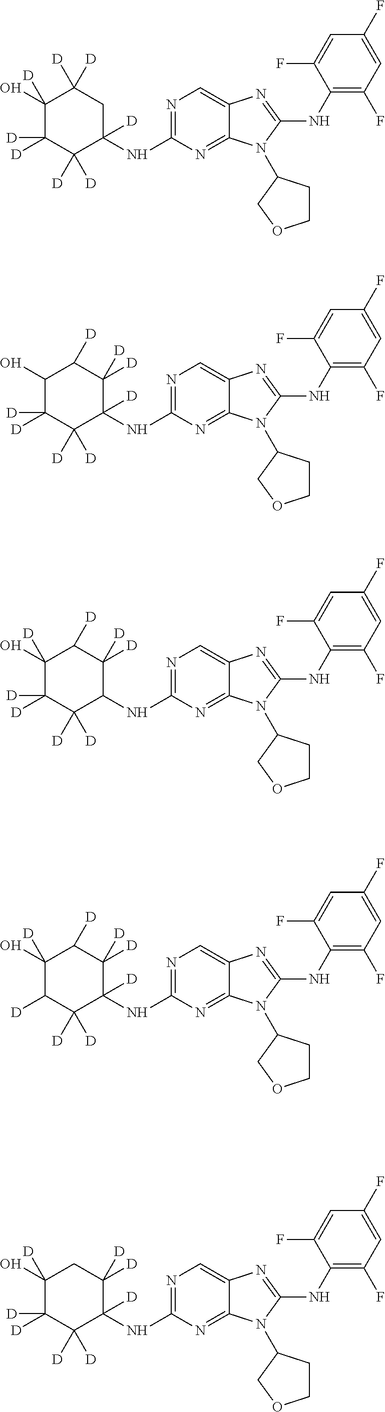

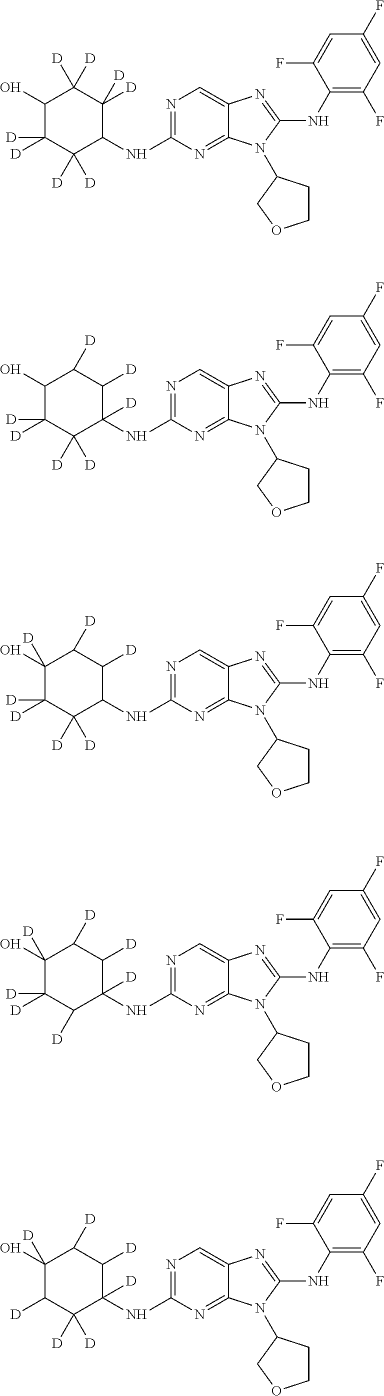 [SMILES: [2H]C1([2H])C(O)C([2H])([2H])C([2H])([2H])C(Nc2ncc3nc(Nc4c(F)cc(F)cc4F)n(C4CCOC4)c3n2)C1([2H])[2H] ; [2H]C1C(O)C([2H])([2H])C([2H])([2H])C([2H])(Nc2ncc3nc(Nc4c(F)cc(F)cc4F)n(C4CCOC4)c3n2)C1[2H] ; [2H]C1C(Nc2ncc3nc(Nc4c(F)cc(F)cc4F)n(C4CCOC4)c3n2)C([2H])([2H])C([2H])([2H])C([2H])(O)C1[2H] ; [2H]C1C([2H])C([2H])(O)C([2H])([2H])C([2H])C1([2H])Nc1ncc2nc(Nc3c(F)cc(F)cc3F)n(C3CCOC3)c2n1 ; [2H]C1C([2H])C([2H])(Nc2ncc3nc(Nc4c(F)cc(F)cc4F)n(C4CCOC4)c3n2)C([2H])([2H])C([2H])C1([2H])O]

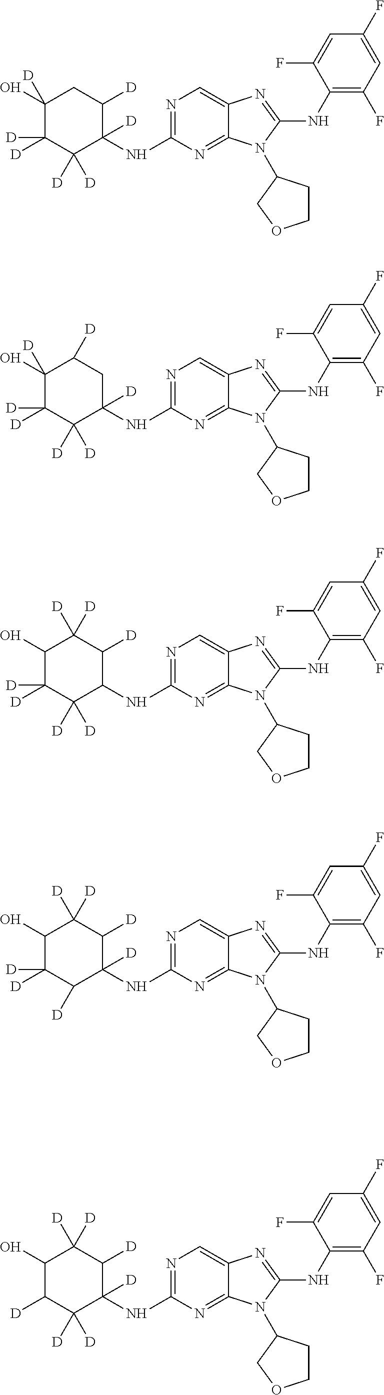 [SMILES: [2H]C1CC([2H])(O)C([2H])([2H])C([2H])([2H])C1([2H])Nc1ncc2nc(Nc3c(F)cc(F)cc3F)n(C3CCOC3)c2n1 ; [2H]C1CC([2H])(Nc2ncc3nc(Nc4c(F)cc(F)cc4F)n(C4CCOC4)c3n2)C([2H])([2H])C([2H])([2H])C1([2H])O ; [2H]C1C(Nc2ncc3nc(Nc4c(F)cc(F)cc4F)n(C4CCOC4)c3n2)C([2H])([2H])C([2H])([2H])C(O)C1([2H])[2H] ; [2H]C1C([2H])([2H])C(O)C([2H])([2H])C([2H])C1([2H])Nc1ncc2nc(Nc3c(F)cc(F)cc3F)n(C3CCOC3)c2n1 ; [2H]C1C(O)C([2H])([2H])C([2H])C([2H])(Nc2ncc3nc(Nc4c(F)cc(F)cc4F)n(C4CCOC4)c3n2)C1([2H])[2H]]

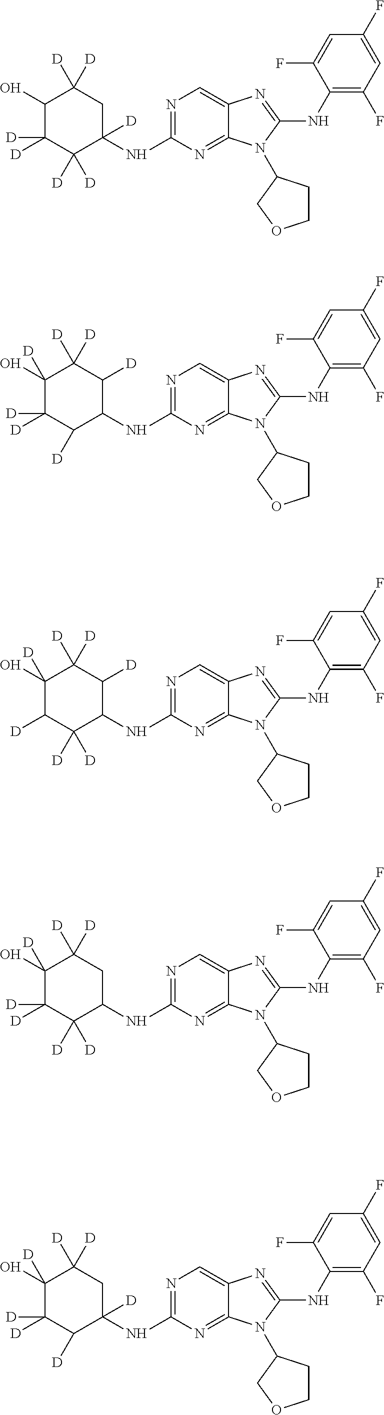 [SMILES: [2H]C1([2H])CC([2H])(Nc2ncc3nc(Nc4c(F)cc(F)cc4F)n(C4CCOC4)c3n2)C([2H])([2H])C([2H])([2H])C1O ; [2H]C1C(Nc2ncc3nc(Nc4c(F)cc(F)cc4F)n(C4CCOC4)c3n2)C([2H])C([2H])([2H])C([2H])(O)C1([2H])[2H] ; [2H]C1C(Nc2ncc3nc(Nc4c(F)cc(F)cc4F)n(C4CCOC4)c3n2)C([2H])([2H])C([2H])C([2H])(O)C1([2H])[2H] ; [2H]C1([2H])CC(Nc2ncc3nc(Nc4c(F)cc(F)cc4F)n(C4CCOC4)c3n2)C([2H])([2H])C([2H])([2H])C1([2H])O ; [2H]C1C([2H])(Nc2ncc3nc(Nc4c(F)cc(F)cc4F)n(C4CCOC4)c3n2)CC([2H])([2H])C([2H])(O)C1([2H])[2H]]

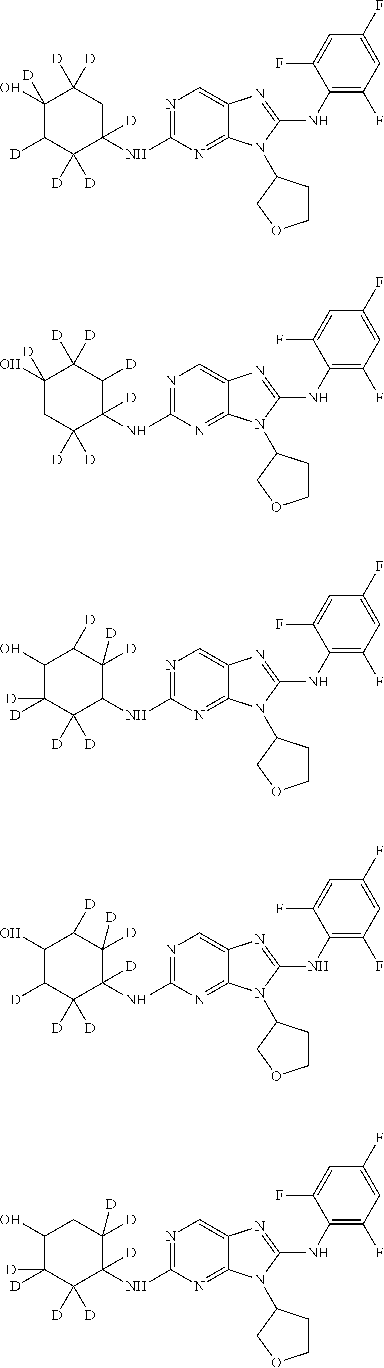 [SMILES: [2H]C1C([2H])([2H])C([2H])(Nc2ncc3nc(Nc4c(F)cc(F)cc4F)n(C4CCOC4)c3n2)CC([2H])([2H])C1([2H])O ; [2H]C1C([2H])([2H])C([2H])(O)CC([2H])([2H])C1([2H])Nc1ncc2nc(Nc3c(F)cc(F)cc3F)n(C3CCOC3)c2n1 ; [2H]C1C(O)C([2H])([2H])C([2H])([2H])C(Nc2ncc3nc(Nc4c(F)cc(F)cc4F)n(C4CCOC4)c3n2)C1([2H])[2H] ; [2H]C1C(O)C([2H])C([2H])([2H])C([2H])(Nc2ncc3nc(Nc4c(F)cc(F)cc4F)n(C4CCOC4)c3n2)C1([2H])[2H] ; [2H]C1([2H])CC(O)C([2H])([2H])C([2H])([2H])C1([2H])Nc1ncc2nc(Nc3c(F)cc(F)cc3F)n(C3CCOC3)c2n1]

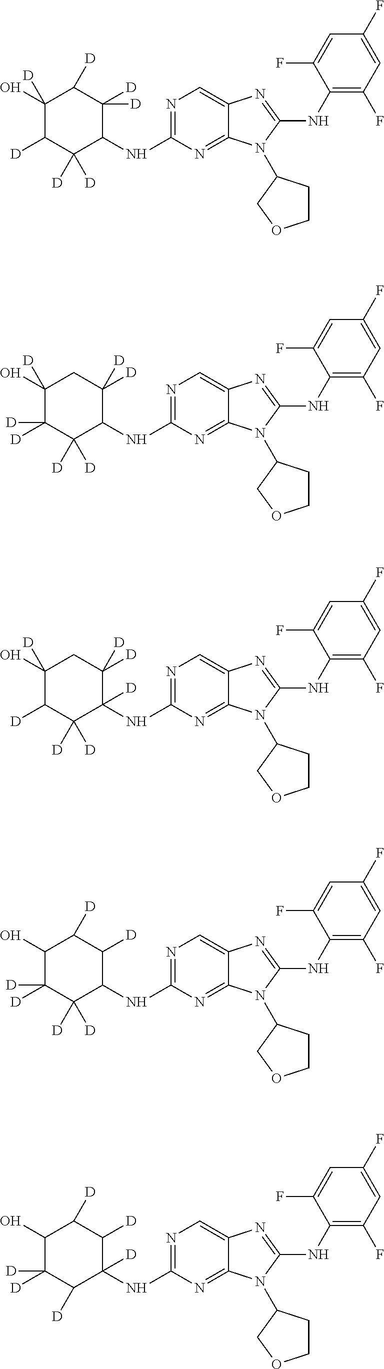 [SMILES: [2H]C1C([2H])([2H])C(Nc2ncc3nc(Nc4c(F)cc(F)cc4F)n(C4CCOC4)c3n2)C([2H])([2H])C([2H])C1([2H])O ; [2H]C1([2H])CC([2H])(O)C([2H])([2H])C([2H])([2H])C1Nc1ncc2nc(Nc3c(F)cc(F)cc3F)n(C3CCOC3)c2n1 ; [2H]C1C([2H])(O)CC([2H])([2H])C([2H])(Nc2ncc3nc(Nc4c(F)cc(F)cc4F)n(C4CCOC4)c3n2)C1([2H])[2H] ; [2H]C1C([2H])C(Nc2ncc3nc(Nc4c(F)cc(F)cc4F)n(C4CCOC4)c3n2)C([2H])([2H])C([2H])([2H])C1O ; [2H]C1C(O)C([2H])([2H])C([2H])C([2H])(Nc2ncc3nc(Nc4c(F)cc(F)cc4F)n(C4CCOC4)c3n2)C1[2H]]

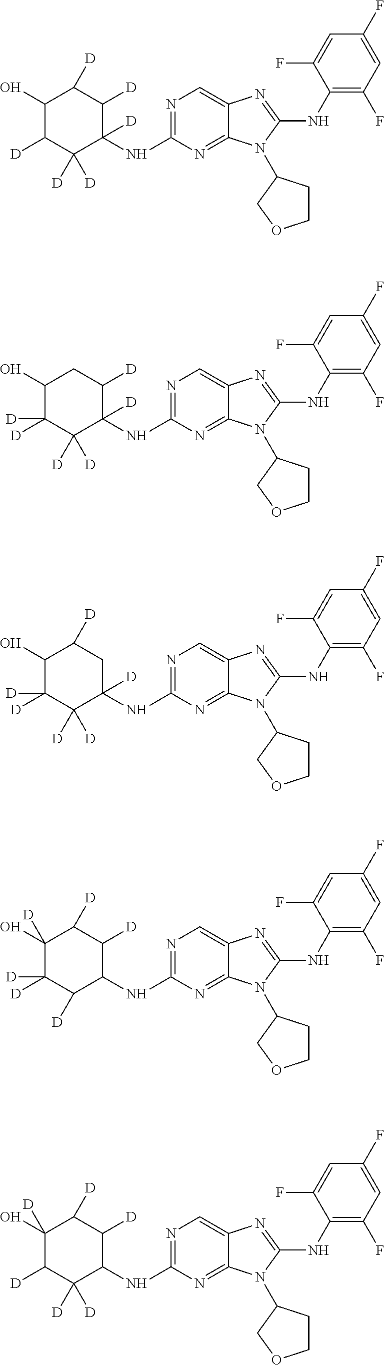 [SMILES: [2H]C1C(O)C([2H])C([2H])([2H])C([2H])(Nc2ncc3nc(Nc4c(F)cc(F)cc4F)n(C4CCOC4)c3n2)C1[2H] ; [2H]C1CC(O)C([2H])([2H])C([2H])([2H])C1([2H])Nc1ncc2nc(Nc3c(F)cc(F)cc3F)n(C3CCOC3)c2n1 ; [2H]C1CC([2H])(Nc2ncc3nc(Nc4c(F)cc(F)cc4F)n(C4CCOC4)c3n2)C([2H])([2H])C([2H])([2H])C1O ; [2H]C1C(Nc2ncc3nc(Nc4c(F)cc(F)cc4F)n(C4CCOC4)c3n2)C([2H])C([2H])([2H])C([2H])(O)C1[2H] ; [2H]C1C(Nc2ncc3nc(Nc4c(F)cc(F)cc4F)n(C4CCOC4)c3n2)C([2H])([2H])C([2H])C([2H])(O)C1[2H]]

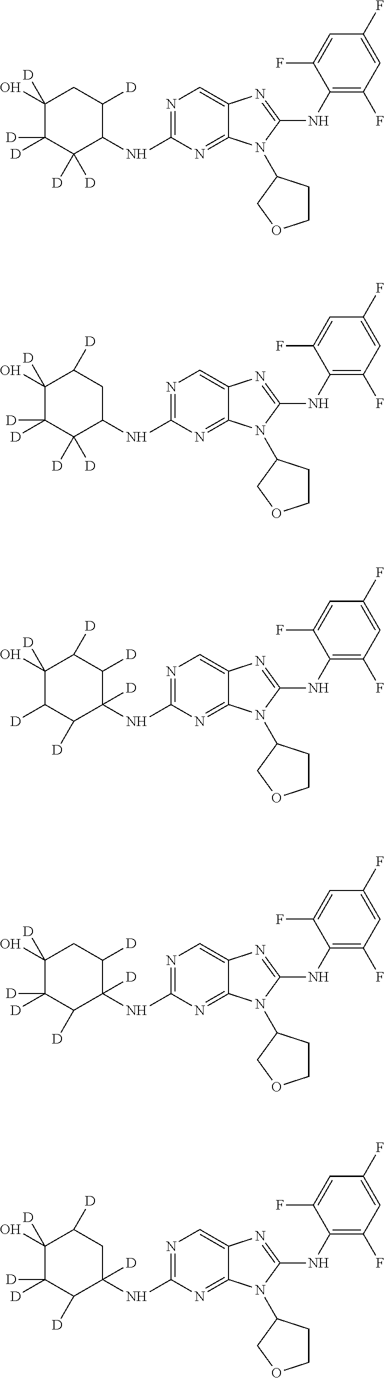 [SMILES: [2H]C1CC([2H])(O)C([2H])([2H])C([2H])([2H])C1Nc1ncc2nc(Nc3c(F)cc(F)cc3F)n(C3CCOC3)c2n1 ; [2H]C1CC(Nc2ncc3nc(Nc4c(F)cc(F)cc4F)n(C4CCOC4)c3n2)C([2H])([2H])C([2H])([2H])C1([2H])O ; [2H]C1C([2H])C([2H])(Nc2ncc3nc(Nc4c(F)cc(F)cc4F)n(C4CCOC4)c3n2)C([2H])C([2H])C1([2H])O ; [2H]C1CC([2H])(O)C([2H])([2H])C([2H])C1([2H])Nc1ncc2nc(Nc3c(F)cc(F)cc3F)n(C3CCOC3)c2n1 ; [2H]C1C([2H])(Nc2ncc3nc(Nc4c(F)cc(F)cc4F)n(C4CCOC4)c3n2)CC([2H])C([2H])(O)C1([2H])[2H]]

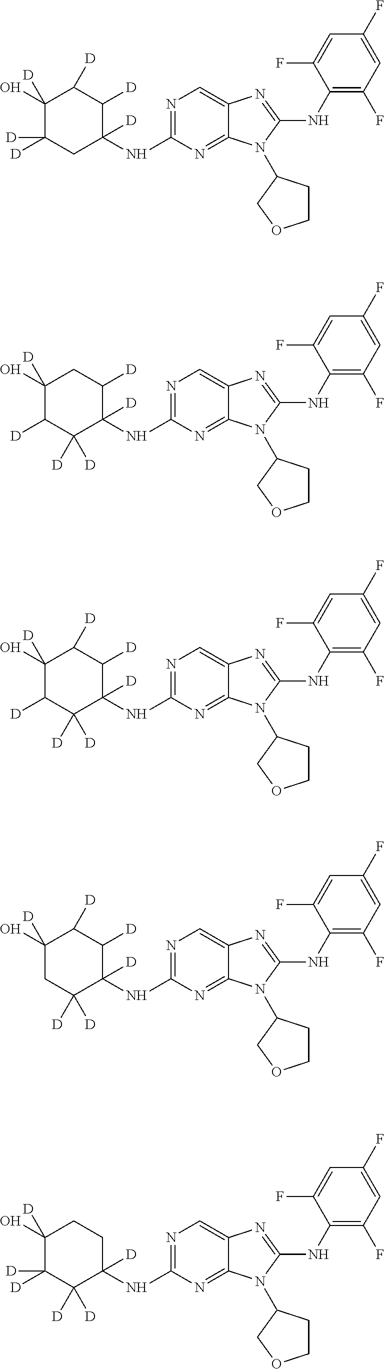 [SMILES: [2H]C1C([2H])C([2H])(O)C([2H])([2H])CC1([2H])Nc1ncc2nc(Nc3c(F)cc(F)cc3F)n(C3CCOC3)c2n1 ; [2H]C1C([2H])(O)CC([2H])C([2H])(Nc2ncc3nc(Nc4c(F)cc(F)cc4F)n(C4CCOC4)c3n2)C1([2H])[2H] ; [2H]C1C([2H])C([2H])(Nc2ncc3nc(Nc4c(F)cc(F)cc4F)n(C4CCOC4)c3n2)C([2H])([2H])C([2H])C1([2H])O ; [2H]C1C([2H])C([2H])(Nc2ncc3nc(Nc4c(F)cc(F)cc4F)n(C4CCOC4)c3n2)C([2H])([2H])CC1([2H])O ; [2H]C1(O)CCC([2H])(Nc2ncc3nc(Nc4c(F)cc(F)cc4F)n(C4CCOC4)c3n2)C([2H])([2H])C1([2H])[2H]]

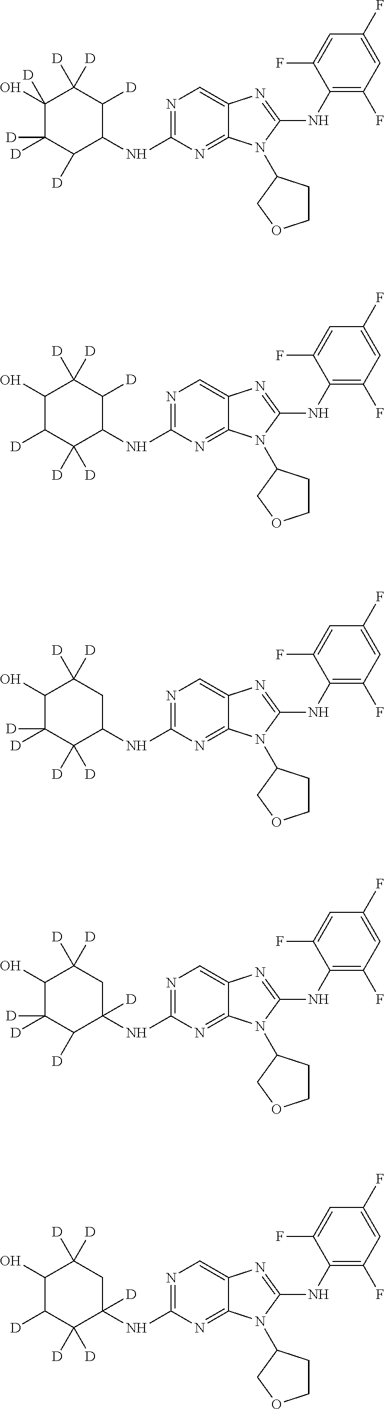 [SMILES: [2H]C1C(Nc2ncc3nc(Nc4c(F)cc(F)cc4F)n(C4CCOC4)c3n2)C([2H])C([2H])([2H])C([2H])(O)C1([2H])[2H] ; [2H]C1C(Nc2ncc3nc(Nc4c(F)cc(F)cc4F)n(C4CCOC4)c3n2)C([2H])([2H])C([2H])C(O)C1([2H])[2H] ; [2H]C1([2H])CC(Nc2ncc3nc(Nc4c(F)cc(F)cc4F)n(C4CCOC4)c3n2)C([2H])([2H])C([2H])([2H])C1O ; [2H]C1C([2H])(Nc2ncc3nc(Nc4c(F)cc(F)cc4F)n(C4CCOC4)c3n2)CC([2H])([2H])C(O)C1([2H])[2H] ; [2H]C1C(O)C([2H])([2H])CC([2H])(Nc2ncc3nc(Nc4c(F)cc(F)cc4F)n(C4CCOC4)c3n2)C1([2H])[2H]]

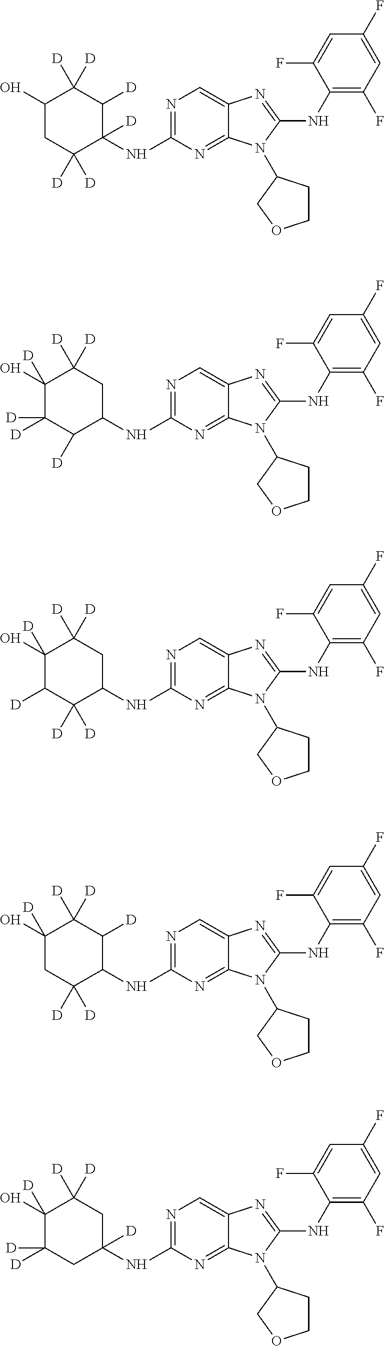 [SMILES: [2H]C1C([2H])([2H])C(O)CC([2H])([2H])C1([2H])Nc1ncc2nc(Nc3c(F)cc(F)cc3F)n(C3CCOC3)c2n1 ; [2H]C1C(Nc2ncc3nc(Nc4c(F)cc(F)cc4F)n(C4CCOC4)c3n2)CC([2H])([2H])C([2H])(O)C1([2H])[2H] ; [2H]C1C([2H])([2H])C(Nc2ncc3nc(Nc4c(F)cc(F)cc4F)n(C4CCOC4)c3n2)CC([2H])([2H])C1([2H])O ; [2H]C1C(Nc2ncc3nc(Nc4c(F)cc(F)cc4F)n(C4CCOC4)c3n2)C([2H])([2H])CC([2H])(O)C1([2H])[2H] ; [2H]C1(Nc2ncc3nc(Nc4c(F)cc(F)cc4F)n(C4CCOC4)c3n2)CC([2H])([2H])C([2H])(O)C([2H])([2H])C1]

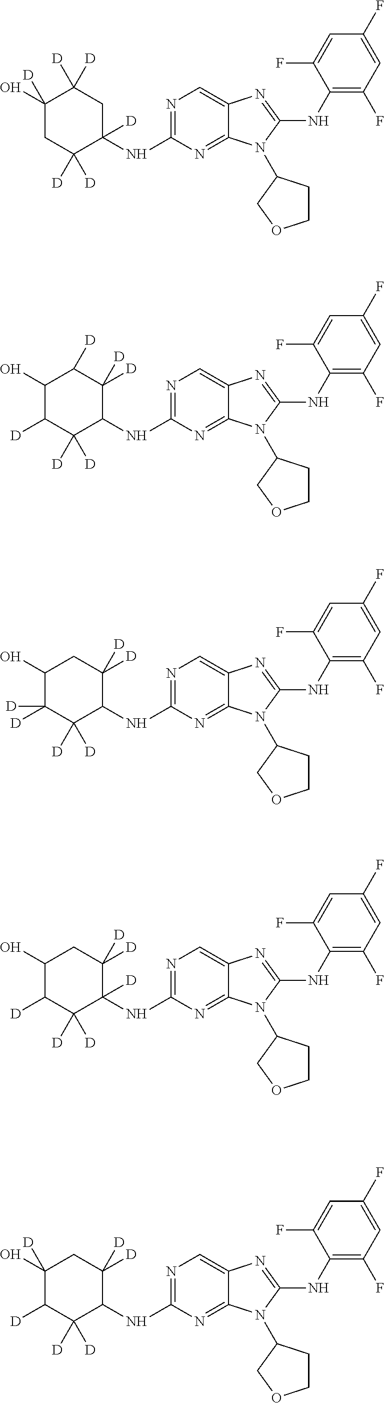 [SMILES: [2H]C1([2H])CC([2H])(Nc2ncc3nc(Nc4c(F)cc(F)cc4F)n(C4CCOC4)c3n2)C([2H])([2H])CC1([2H])O ; [2H]C1C(O)C([2H])C([2H])([2H])C(Nc2ncc3nc(Nc4c(F)cc(F)cc4F)n(C4CCOC4)c3n2)C1([2H])[2H] ; [2H]C1([2H])CC(O)C([2H])([2H])C([2H])([2H])C1Nc1ncc2nc(Nc3c(F)cc(F)cc3F)n(C3CCOC3)c2n1 ; [2H]C1C(O)CC([2H])([2H])C([2H])(Nc2ncc3nc(Nc4c(F)cc(F)cc4F)n(C4CCOC4)c3n2)C1([2H])[2H] ; [2H]C1C([2H])(O)CC([2H])([2H])C(Nc2ncc3nc(Nc4c(F)cc(F)cc4F)n(C4CCOC4)c3n2)C1([2H])[2H]]

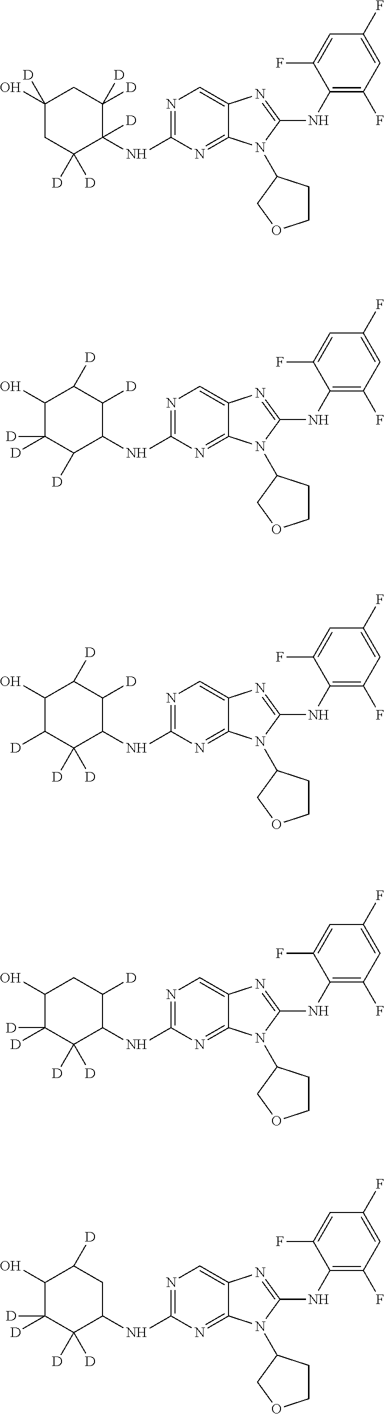 [SMILES: [2H]C1(O)CC([2H])([2H])C([2H])(Nc2ncc3nc(Nc4c(F)cc(F)cc4F)n(C4CCOC4)c3n2)C([2H])([2H])C1 ; [2H]C1C([2H])C(O)C([2H])([2H])C([2H])C1Nc1ncc2nc(Nc3c(F)cc(F)cc3F)n(C3CCOC3)c2n1 ; [2H]C1C([2H])C(Nc2ncc3nc(Nc4c(F)cc(F)cc4F)n(C4CCOC4)c3n2)C([2H])([2H])C([2H])C1O ; [2H]C1CC(O)C([2H])([2H])C([2H])([2H])C1Nc1ncc2nc(Nc3c(F)cc(F)cc3F)n(C3CCOC3)c2n1 ; [2H]C1CC(Nc2ncc3nc(Nc4c(F)cc(F)cc4F)n(C4CCOC4)c3n2)C([2H])([2H])C([2H])([2H])C1O]

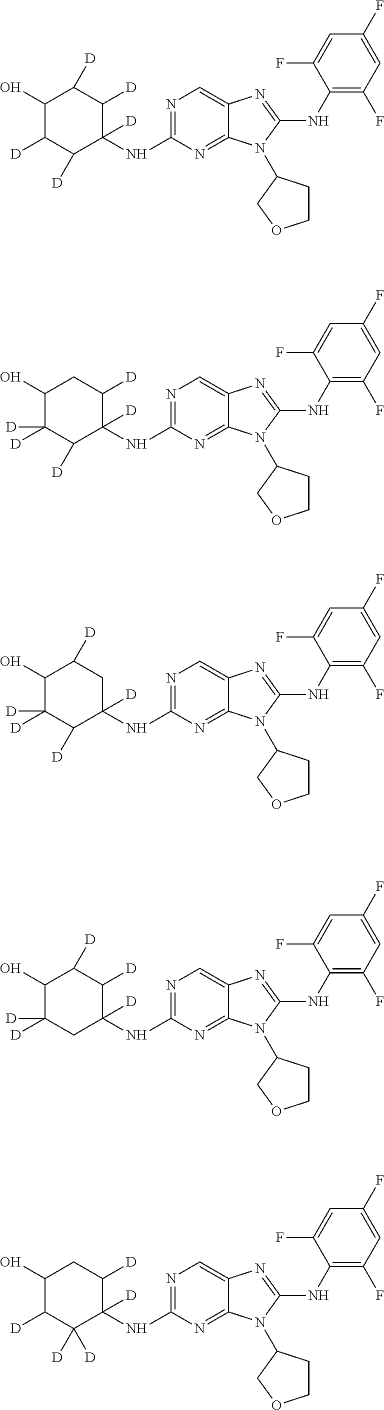 [SMILES: [2H]C1C(O)C([2H])C([2H])C([2H])(Nc2ncc3nc(Nc4c(F)cc(F)cc4F)n(C4CCOC4)c3n2)C1[2H] ; [2H]C1CC(O)C([2H])([2H])C([2H])C1([2H])Nc1ncc2nc(Nc3c(F)cc(F)cc3F)n(C3CCOC3)c2n1 ; [2H]C1CC([2H])(Nc2ncc3nc(Nc4c(F)cc(F)cc4F)n(C4CCOC4)c3n2)C([2H])C([2H])([2H])C1O ; [2H]C1C(O)C([2H])([2H])CC([2H])(Nc2ncc3nc(Nc4c(F)cc(F)cc4F)n(C4CCOC4)c3n2)C1[2H] ; [2H]C1C(O)CC([2H])C([2H])(Nc2ncc3nc(Nc4c(F)cc(F)cc4F)n(C4CCOC4)c3n2)C1([2H])[2H]]

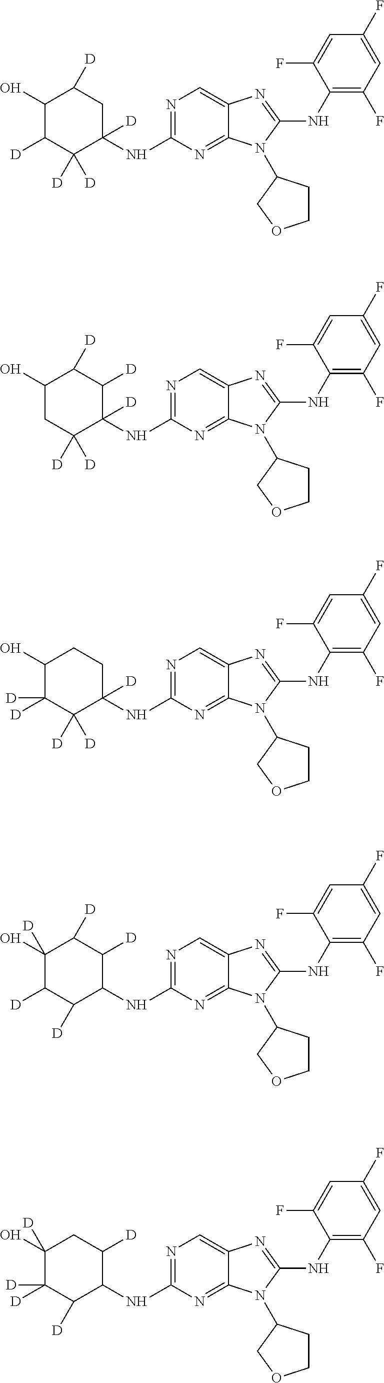 [SMILES: [2H]C1CC([2H])(Nc2ncc3nc(Nc4c(F)cc(F)cc4F)n(C4CCOC4)c3n2)C([2H])([2H])C([2H])C1O ; [2H]C1C(O)CC([2H])([2H])C([2H])(Nc2ncc3nc(Nc4c(F)cc(F)cc4F)n(C4CCOC4)c3n2)C1[2H] ; [2H]C1(Nc2ncc3nc(Nc4c(F)cc(F)cc4F)n(C4CCOC4)c3n2)CCC(O)C([2H])([2H])C1([2H])[2H] ; [2H]C1C(Nc2ncc3nc(Nc4c(F)cc(F)cc4F)n(C4CCOC4)c3n2)C([2H])C([2H])C([2H])(O)C1[2H] ; [2H]C1CC([2H])(O)C([2H])([2H])C([2H])C1Nc1ncc2nc(Nc3c(F)cc(F)cc3F)n(C3CCOC3)c2n1]

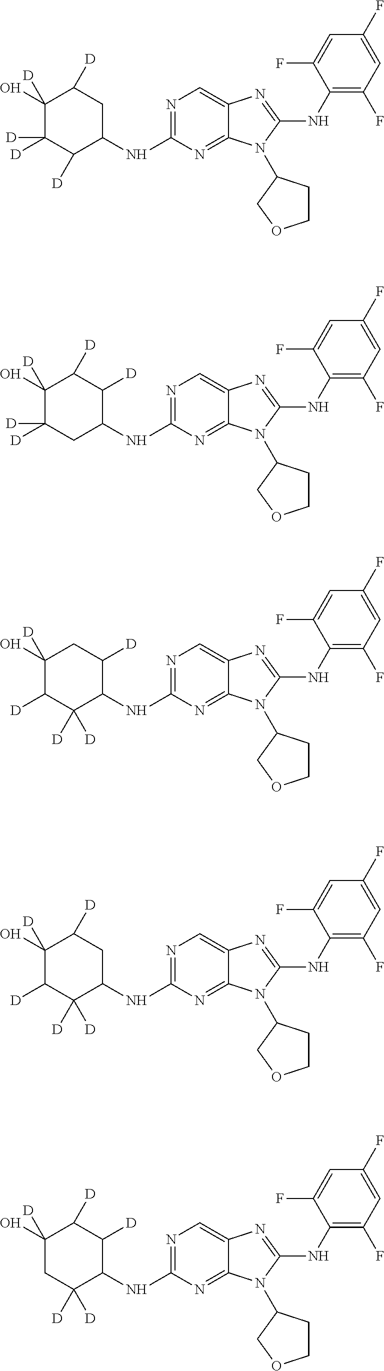 [SMILES: [2H]C1C(Nc2ncc3nc(Nc4c(F)cc(F)cc4F)n(C4CCOC4)c3n2)CC([2H])C([2H])(O)C1([2H])[2H] ; [2H]C1C(Nc2ncc3nc(Nc4c(F)cc(F)cc4F)n(C4CCOC4)c3n2)CC([2H])([2H])C([2H])(O)C1[2H] ; [2H]C1CC([2H])(O)C([2H])C([2H])([2H])C1Nc1ncc2nc(Nc3c(F)cc(F)cc3F)n(C3CCOC3)c2n1 ; [2H]C1CC(Nc2ncc3nc(Nc4c(F)cc(F)cc4F)n(C4CCOC4)c3n2)C([2H])([2H])C([2H])C1([2H])O ; [2H]C1C(Nc2ncc3nc(Nc4c(F)cc(F)cc4F)n(C4CCOC4)c3n2)C([2H])([2H])CC([2H])(O)C1[2H]]

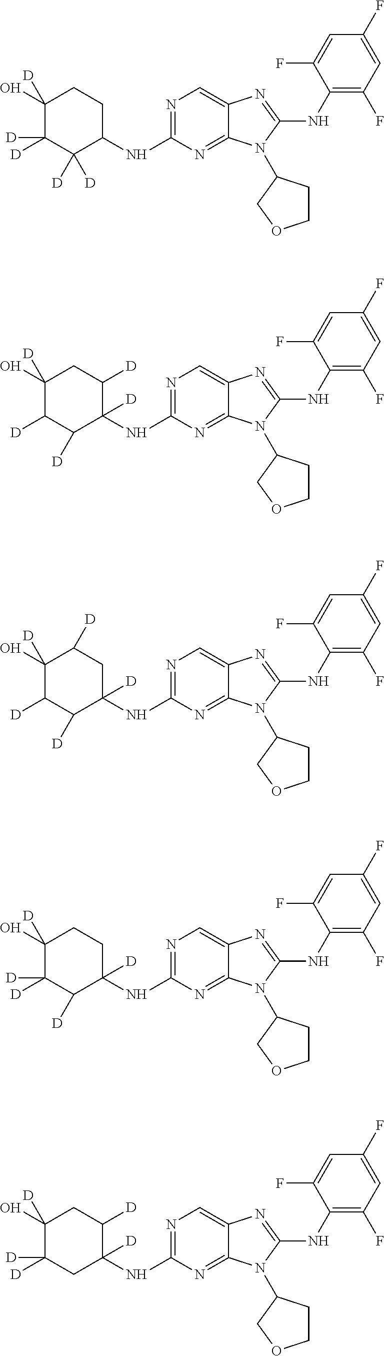 [SMILES: [2H]C1(O)CCC(Nc2ncc3nc(Nc4c(F)cc(F)cc4F)n(C4CCOC4)c3n2)C([2H])([2H])C1([2H])[2H] ; [2H]C1C([2H])C([2H])(Nc2ncc3nc(Nc4c(F)cc(F)cc4F)n(C4CCOC4)c3n2)C([2H])CC1([2H])O ; [2H]C1C([2H])C([2H])(O)C([2H])CC1([2H])Nc1ncc2nc(Nc3c(F)cc(F)cc3F)n(C3CCOC3)c2n1 ; [2H]C1C([2H])(Nc2ncc3nc(Nc4c(F)cc(F)cc4F)n(C4CCOC4)c3n2)CCC([2H])(O)C1([2H])[2H] ; [2H]C1CC([2H])(O)C([2H])([2H])CC1([2H])Nc1ncc2nc(Nc3c(F)cc(F)cc3F)n(C3CCOC3)c2n1]

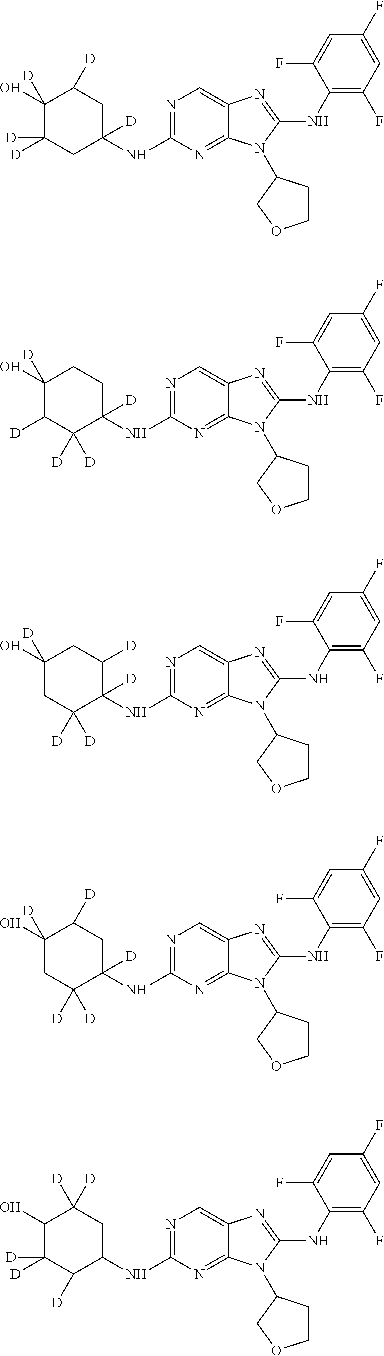 [SMILES: [2H]C1CC([2H])(Nc2ncc3nc(Nc4c(F)cc(F)cc4F)n(C4CCOC4)c3n2)CC([2H])([2H])C1([2H])O ; [2H]C1C([2H])(O)CCC([2H])(Nc2ncc3nc(Nc4c(F)cc(F)cc4F)n(C4CCOC4)c3n2)C1([2H])[2H] ; [2H]C1CC([2H])(O)CC([2H])([2H])C1([2H])Nc1ncc2nc(Nc3c(F)cc(F)cc3F)n(C3CCOC3)c2n1 ; [2H]C1CC([2H])(Nc2ncc3nc(Nc4c(F)cc(F)cc4F)n(C4CCOC4)c3n2)C([2H])([2H])CC1([2H])O ; [2H]C1C(Nc2ncc3nc(Nc4c(F)cc(F)cc4F)n(C4CCOC4)c3n2)CC([2H])([2H])C(O)C1([2H])[2H]]

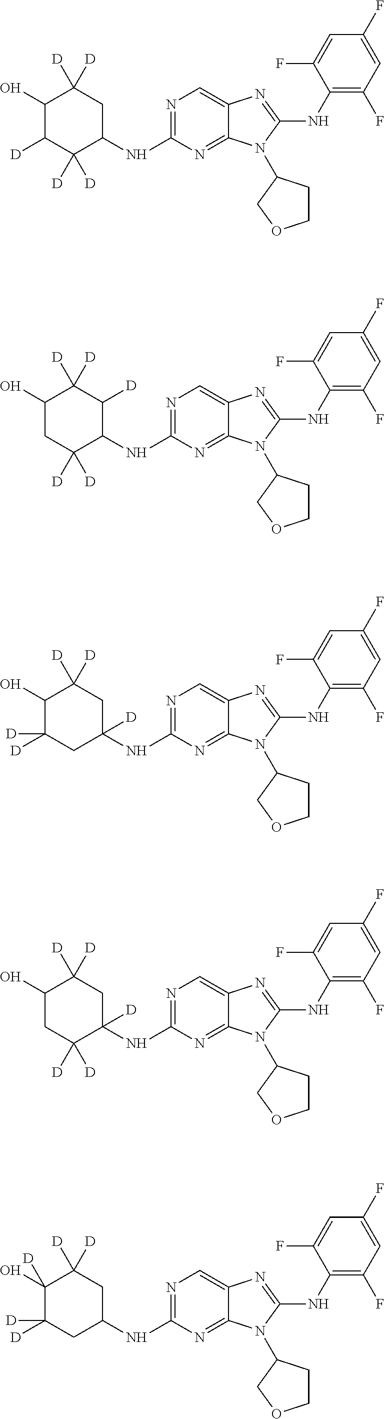 [SMILES: [2H]C1C(O)C([2H])([2H])CC(Nc2ncc3nc(Nc4c(F)cc(F)cc4F)n(C4CCOC4)c3n2)C1([2H])[2H] ; [2H]C1C(Nc2ncc3nc(Nc4c(F)cc(F)cc4F)n(C4CCOC4)c3n2)C([2H])([2H])CC(O)C1([2H])[2H] ; [2H]C1(Nc2ncc3nc(Nc4c(F)cc(F)cc4F)n(C4CCOC4)c3n2)CC([2H])([2H])C(O)C([2H])([2H])C1 ; [2H]C1([2H])CC([2H])(Nc2ncc3nc(Nc4c(F)cc(F)cc4F)n(C4CCOC4)c3n2)C([2H])([2H])CC1O ; [2H]C1([2H])CC(Nc2ncc3nc(Nc4c(F)cc(F)cc4F)n(C4CCOC4)c3n2)CC([2H])([2H])C1([2H])O]

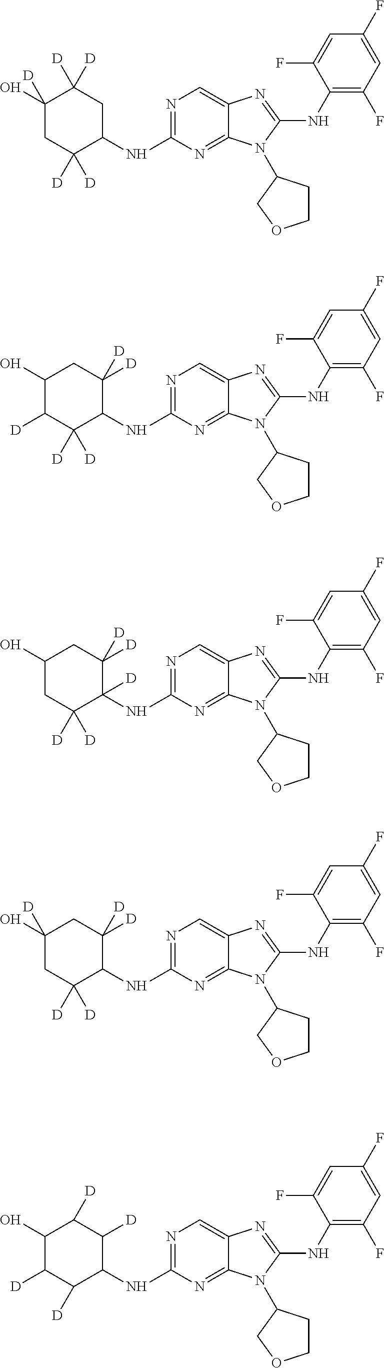 [SMILES: [2H]C1([2H])CC([2H])(O)C([2H])([2H])CC1Nc1ncc2nc(Nc3c(F)cc(F)cc3F)n(C3CCOC3)c2n1 ; [2H]C1C(O)CC([2H])([2H])C(Nc2ncc3nc(Nc4c(F)cc(F)cc4F)n(C4CCOC4)c3n2)C1([2H])[2H] ; [2H]C1([2H])CC(O)CC([2H])([2H])C1([2H])Nc1ncc2nc(Nc3c(F)cc(F)cc3F)n(C3CCOC3)c2n1 ; [2H]C1(O)CC([2H])([2H])C(Nc2ncc3nc(Nc4c(F)cc(F)cc4F)n(C4CCOC4)c3n2)C([2H])([2H])C1 ; [2H]C1C([2H])C(Nc2ncc3nc(Nc4c(F)cc(F)cc4F)n(C4CCOC4)c3n2)C([2H])C([2H])C1O]

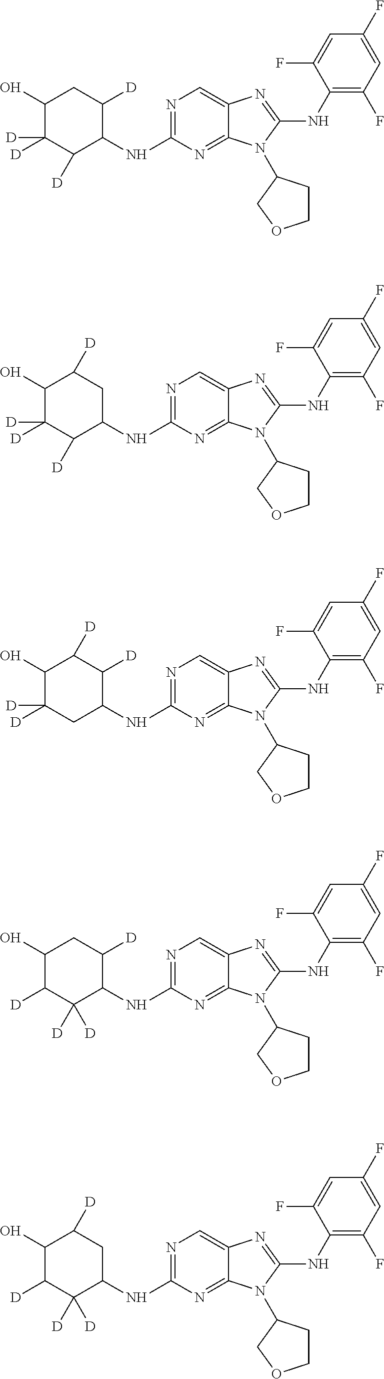 [SMILES: [2H]C1CC(O)C([2H])([2H])C([2H])C1Nc1ncc2nc(Nc3c(F)cc(F)cc3F)n(C3CCOC3)c2n1 ; [2H]C1CC(Nc2ncc3nc(Nc4c(F)cc(F)cc4F)n(C4CCOC4)c3n2)C([2H])C([2H])([2H])C1O ; [2H]C1C(Nc2ncc3nc(Nc4c(F)cc(F)cc4F)n(C4CCOC4)c3n2)CC([2H])([2H])C(O)C1[2H] ; [2H]C1CC(O)C([2H])C([2H])([2H])C1Nc1ncc2nc(Nc3c(F)cc(F)cc3F)n(C3CCOC3)c2n1 ; [2H]C1CC(Nc2ncc3nc(Nc4c(F)cc(F)cc4F)n(C4CCOC4)c3n2)C([2H])([2H])C([2H])C1O]

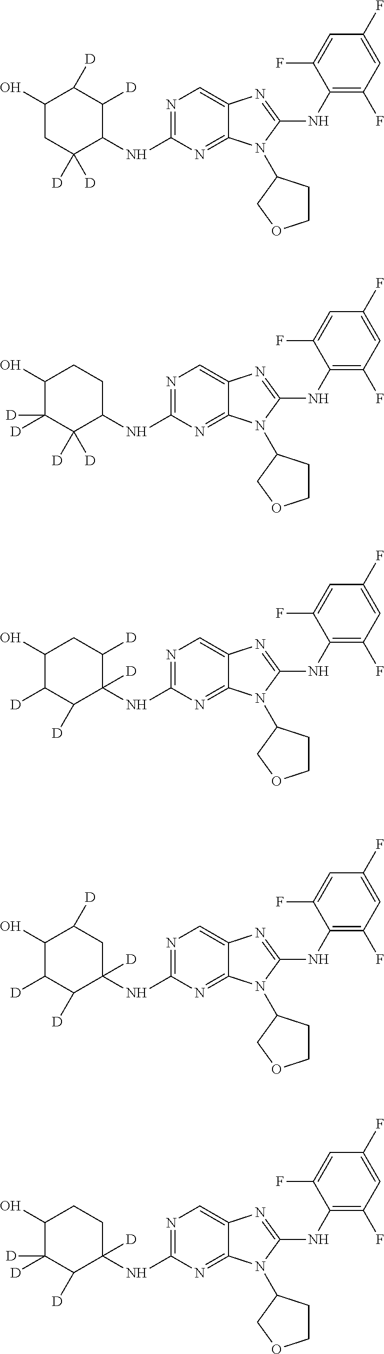 [SMILES: [2H]C1C(O)CC([2H])([2H])C(Nc2ncc3nc(Nc4c(F)cc(F)cc4F)n(C4CCOC4)c3n2)C1[2H] ; [2H]C1([2H])C(O)CCC(Nc2ncc3nc(Nc4c(F)cc(F)cc4F)n(C4CCOC4)c3n2)C1([2H])[2H] ; [2H]C1C(O)CC([2H])C([2H])(Nc2ncc3nc(Nc4c(F)cc(F)cc4F)n(C4CCOC4)c3n2)C1[2H] ; [2H]C1CC([2H])(Nc2ncc3nc(Nc4c(F)cc(F)cc4F)n(C4CCOC4)c3n2)C([2H])C([2H])C1O ; [2H]C1C([2H])(Nc2ncc3nc(Nc4c(F)cc(F)cc4F)n(C4CCOC4)c3n2)CCC(O)C1([2H])[2H]]

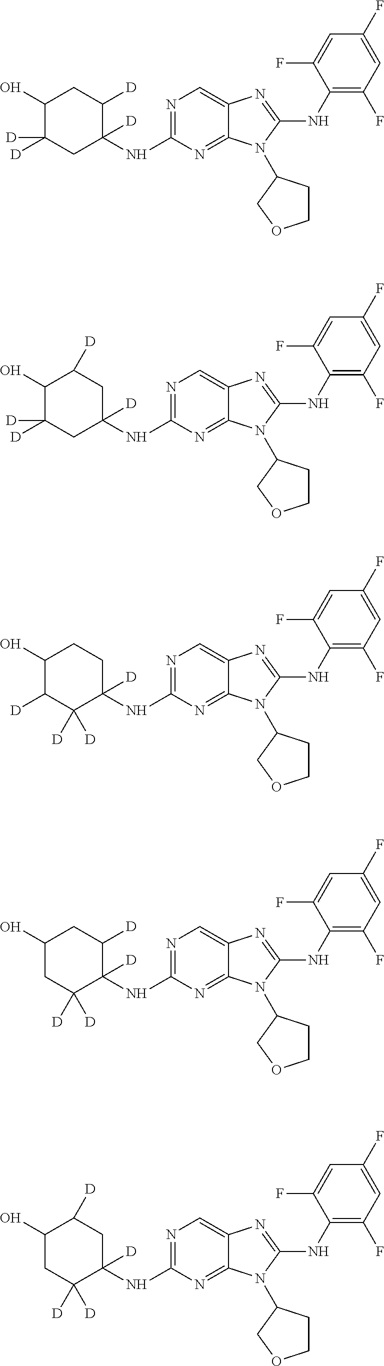 [SMILES: [2H]C1CC(O)C([2H])([2H])CC1([2H])Nc1ncc2nc(Nc3c(F)cc(F)cc3F)n(C3CCOC3)c2n1 ; [2H]C1CC([2H])(Nc2ncc3nc(Nc4c(F)cc(F)cc4F)n(C4CCOC4)c3n2)CC([2H])([2H])C1O ; [2H]C1C(O)CCC([2H])(Nc2ncc3nc(Nc4c(F)cc(F)cc4F)n(C4CCOC4)c3n2)C1([2H])[2H] ; [2H]C1CC(O)CC([2H])([2H])C1([2H])Nc1ncc2nc(Nc3c(F)cc(F)cc3F)n(C3CCOC3)c2n1 ; [2H]C1CC([2H])(Nc2ncc3nc(Nc4c(F)cc(F)cc4F)n(C4CCOC4)c3n2)C([2H])([2H])CC1O]

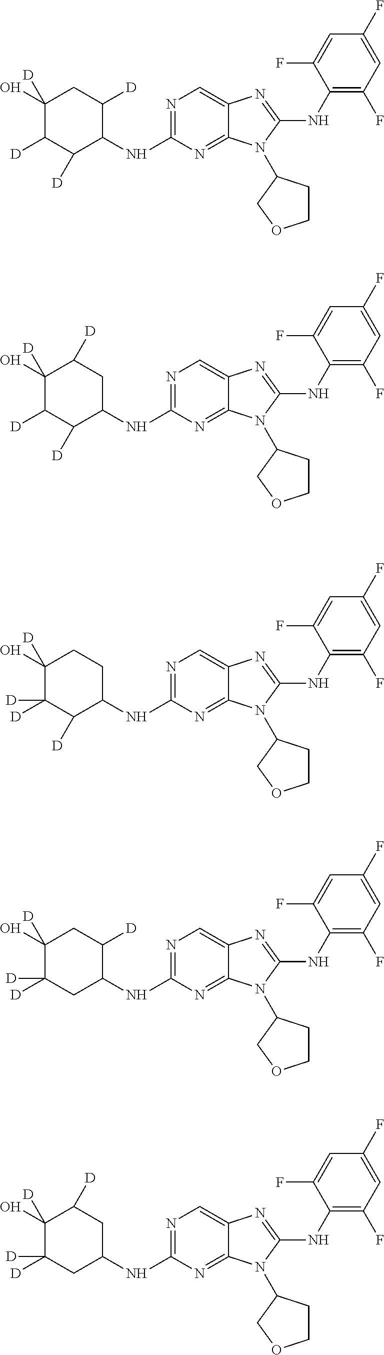 [SMILES: [2H]C1CC([2H])(O)C([2H])C([2H])C1Nc1ncc2nc(Nc3c(F)cc(F)cc3F)n(C3CCOC3)c2n1 ; [2H]C1C(Nc2ncc3nc(Nc4c(F)cc(F)cc4F)n(C4CCOC4)c3n2)CC([2H])C([2H])(O)C1[2H] ; [2H]C1C(Nc2ncc3nc(Nc4c(F)cc(F)cc4F)n(C4CCOC4)c3n2)CCC([2H])(O)C1([2H])[2H] ; [2H]C1CC([2H])(O)C([2H])([2H])CC1Nc1ncc2nc(Nc3c(F)cc(F)cc3F)n(C3CCOC3)c2n1 ; [2H]C1CC(Nc2ncc3nc(Nc4c(F)cc(F)cc4F)n(C4CCOC4)c3n2)CC([2H])([2H])C1([2H])O]

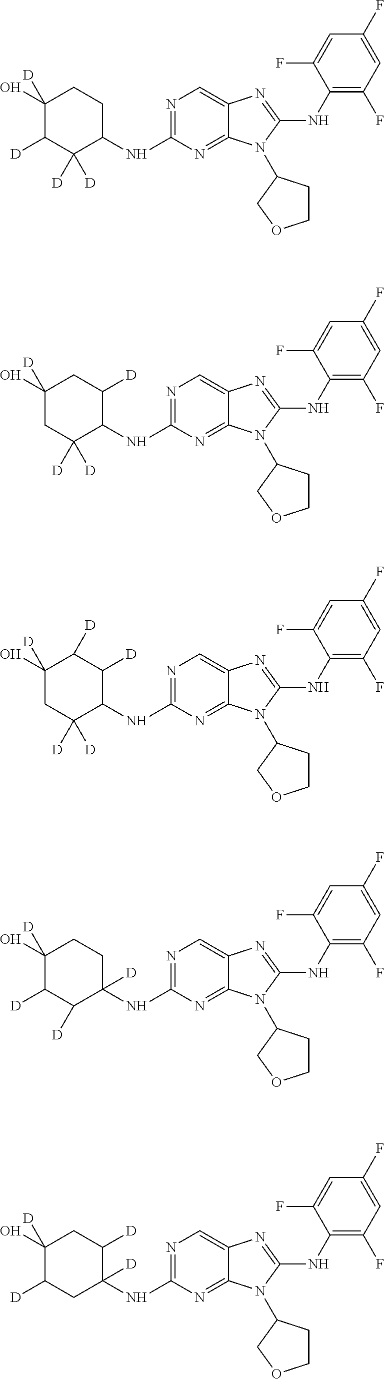 [SMILES: [2H]C1C([2H])(O)CCC(Nc2ncc3nc(Nc4c(F)cc(F)cc4F)n(C4CCOC4)c3n2)C1([2H])[2H] ; [2H]C1CC([2H])(O)CC([2H])([2H])C1Nc1ncc2nc(Nc3c(F)cc(F)cc3F)n(C3CCOC3)c2n1 ; [2H]C1C(Nc2ncc3nc(Nc4c(F)cc(F)cc4F)n(C4CCOC4)c3n2)C([2H])([2H])CC([2H])(O)C1[2H] ; [2H]C1C([2H])C([2H])(Nc2ncc3nc(Nc4c(F)cc(F)cc4F)n(C4CCOC4)c3n2)CCC1([2H])O ; [2H]C1CC([2H])(Nc2ncc3nc(Nc4c(F)cc(F)cc4F)n(C4CCOC4)c3n2)C([2H])CC1([2H])O]

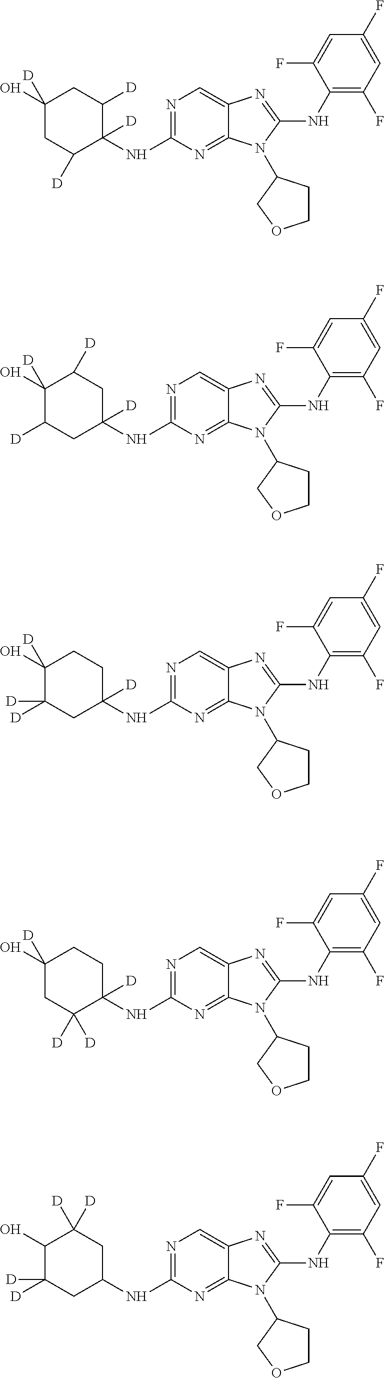 [SMILES: [2H]C1CC([2H])(O)CC([2H])C1([2H])Nc1ncc2nc(Nc3c(F)cc(F)cc3F)n(C3CCOC3)c2n1 ; [2H]C1CC([2H])(Nc2ncc3nc(Nc4c(F)cc(F)cc4F)n(C4CCOC4)c3n2)CC([2H])C1([2H])O ; [2H]C1(Nc2ncc3nc(Nc4c(F)cc(F)cc4F)n(C4CCOC4)c3n2)CCC([2H])(O)C([2H])([2H])C1 ; [2H]C1(O)CCC([2H])(Nc2ncc3nc(Nc4c(F)cc(F)cc4F)n(C4CCOC4)c3n2)C([2H])([2H])C1 ; [2H]C1([2H])CC(Nc2ncc3nc(Nc4c(F)cc(F)cc4F)n(C4CCOC4)c3n2)CC([2H])([2H])C1O]

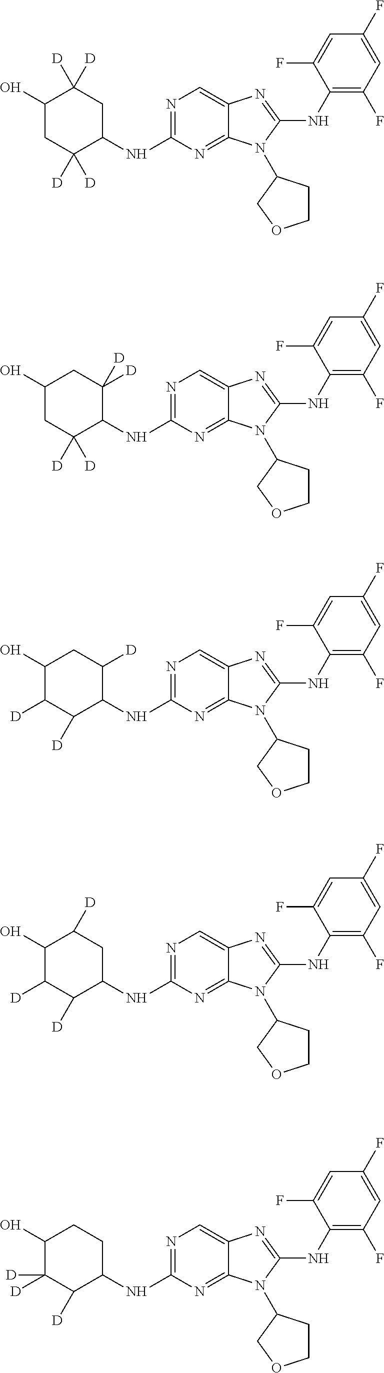 [SMILES: [2H]C1([2H])CC(Nc2ncc3nc(Nc4c(F)cc(F)cc4F)n(C4CCOC4)c3n2)C([2H])([2H])CC1O ; [2H]C1([2H])CC(O)CC([2H])([2H])C1Nc1ncc2nc(Nc3c(F)cc(F)cc3F)n(C3CCOC3)c2n1 ; [2H]C1CC(O)C([2H])C([2H])C1Nc1ncc2nc(Nc3c(F)cc(F)cc3F)n(C3CCOC3)c2n1 ; [2H]C1CC(Nc2ncc3nc(Nc4c(F)cc(F)cc4F)n(C4CCOC4)c3n2)C([2H])C([2H])C1O ; [2H]C1C(Nc2ncc3nc(Nc4c(F)cc(F)cc4F)n(C4CCOC4)c3n2)CCC(O)C1([2H])[2H]]

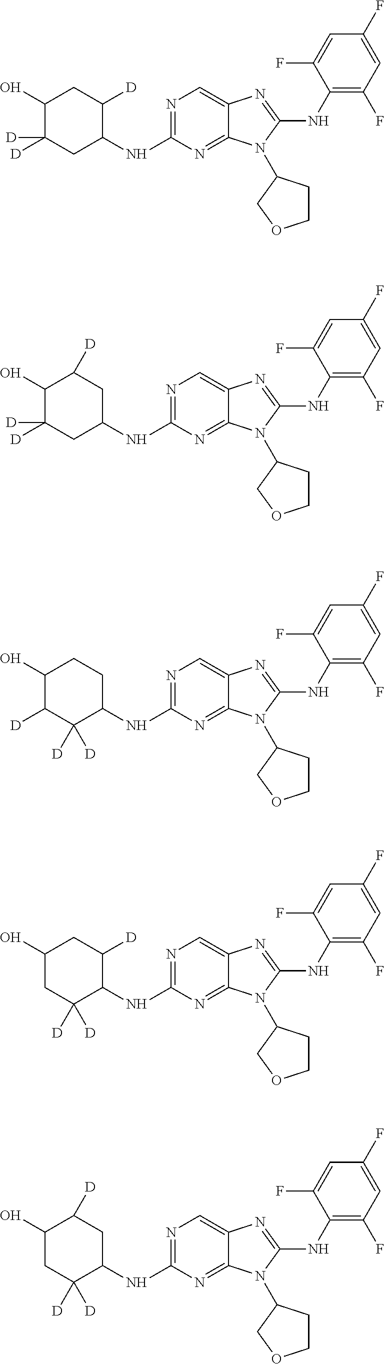 [SMILES: [2H]C1CC(O)C([2H])([2H])CC1Nc1ncc2nc(Nc3c(F)cc(F)cc3F)n(C3CCOC3)c2n1 ; [2H]C1CC(Nc2ncc3nc(Nc4c(F)cc(F)cc4F)n(C4CCOC4)c3n2)CC([2H])([2H])C1O ; [2H]C1C(O)CCC(Nc2ncc3nc(Nc4c(F)cc(F)cc4F)n(C4CCOC4)c3n2)C1([2H])[2H] ; [2H]C1CC(O)CC([2H])([2H])C1Nc1ncc2nc(Nc3c(F)cc(F)cc3F)n(C3CCOC3)c2n1 ; [2H]C1CC(Nc2ncc3nc(Nc4c(F)cc(F)cc4F)n(C4CCOC4)c3n2)C([2H])([2H])CC1O]

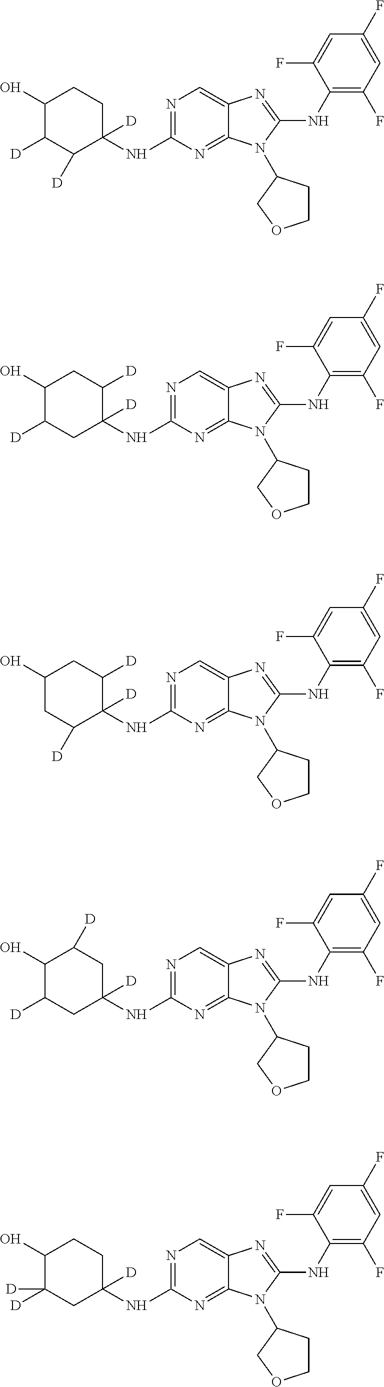 [SMILES: [2H]C1C(O)CCC([2H])(Nc2ncc3nc(Nc4c(F)cc(F)cc4F)n(C4CCOC4)c3n2)C1[2H] ; [2H]C1CC([2H])(Nc2ncc3nc(Nc4c(F)cc(F)cc4F)n(C4CCOC4)c3n2)C([2H])CC1O ; [2H]C1CC(O)CC([2H])C1([2H])Nc1ncc2nc(Nc3c(F)cc(F)cc3F)n(C3CCOC3)c2n1 ; [2H]C1CC([2H])(Nc2ncc3nc(Nc4c(F)cc(F)cc4F)n(C4CCOC4)c3n2)CC([2H])C1O ; [2H]C1(Nc2ncc3nc(Nc4c(F)cc(F)cc4F)n(C4CCOC4)c3n2)CCC(O)C([2H])([2H])C1]

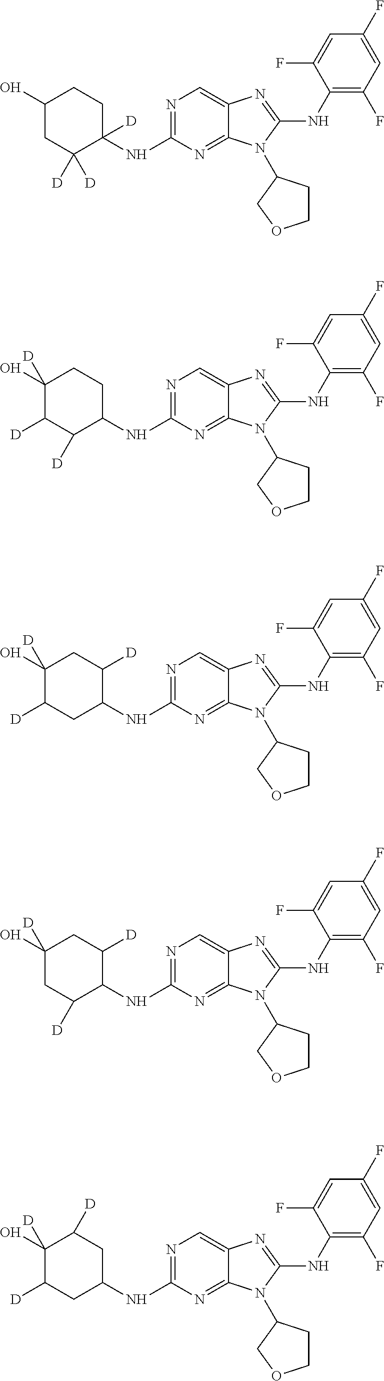 [SMILES: [2H]C1([2H])CC(O)CCC1([2H])Nc1ncc2nc(Nc3c(F)cc(F)cc3F)n(C3CCOC3)c2n1 ; [2H]C1C(Nc2ncc3nc(Nc4c(F)cc(F)cc4F)n(C4CCOC4)c3n2)CCC([2H])(O)C1[2H] ; [2H]C1CC([2H])(O)C([2H])CC1Nc1ncc2nc(Nc3c(F)cc(F)cc3F)n(C3CCOC3)c2n1 ; [2H]C1CC([2H])(O)CC([2H])C1Nc1ncc2nc(Nc3c(F)cc(F)cc3F)n(C3CCOC3)c2n1 ; [2H]C1CC(Nc2ncc3nc(Nc4c(F)cc(F)cc4F)n(C4CCOC4)c3n2)CC([2H])C1([2H])O]

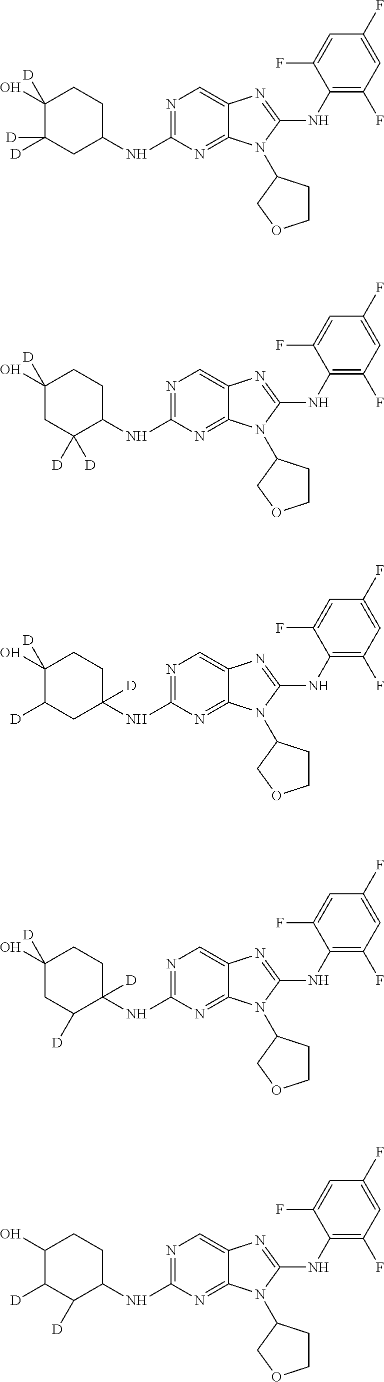 [SMILES: [2H]C1([2H])CC(Nc2ncc3nc(Nc4c(F)cc(F)cc4F)n(C4CCOC4)c3n2)CCC1([2H])O ; [2H]C1(O)CCC(Nc2ncc3nc(Nc4c(F)cc(F)cc4F)n(C4CCOC4)c3n2)C([2H])([2H])C1 ; [2H]C1CC([2H])(Nc2ncc3nc(Nc4c(F)cc(F)cc4F)n(C4CCOC4)c3n2)CCC1([2H])O ; [2H]C1CC([2H])(O)CCC1([2H])Nc1ncc2nc(Nc3c(F)cc(F)cc3F)n(C3CCOC3)c2n1 ; [2H]C1C(O)CCC(Nc2ncc3nc(Nc4c(F)cc(F)cc4F)n(C4CCOC4)c3n2)C1[2H]]

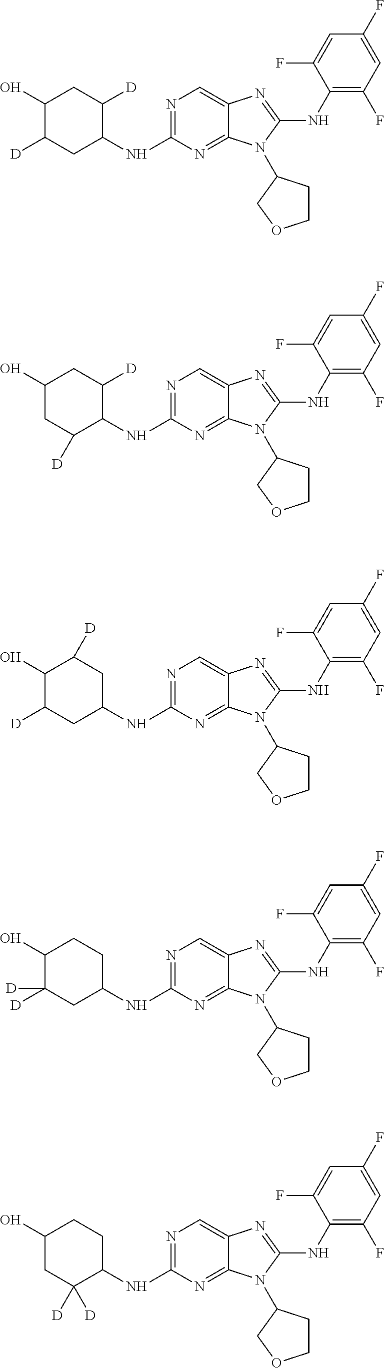 [SMILES: [2H]C1CC(Nc2ncc3nc(Nc4c(F)cc(F)cc4F)n(C4CCOC4)c3n2)C([2H])CC1O ; [2H]C1CC(O)CC([2H])C1Nc1ncc2nc(Nc3c(F)cc(F)cc3F)n(C3CCOC3)c2n1 ; [2H]C1CC(Nc2ncc3nc(Nc4c(F)cc(F)cc4F)n(C4CCOC4)c3n2)CC([2H])C1O ; [2H]C1([2H])CC(Nc2ncc3nc(Nc4c(F)cc(F)cc4F)n(C4CCOC4)c3n2)CCC1O ; [2H]C1([2H])CC(O)CCC1Nc1ncc2nc(Nc3c(F)cc(F)cc3F)n(C3CCOC3)c2n1]

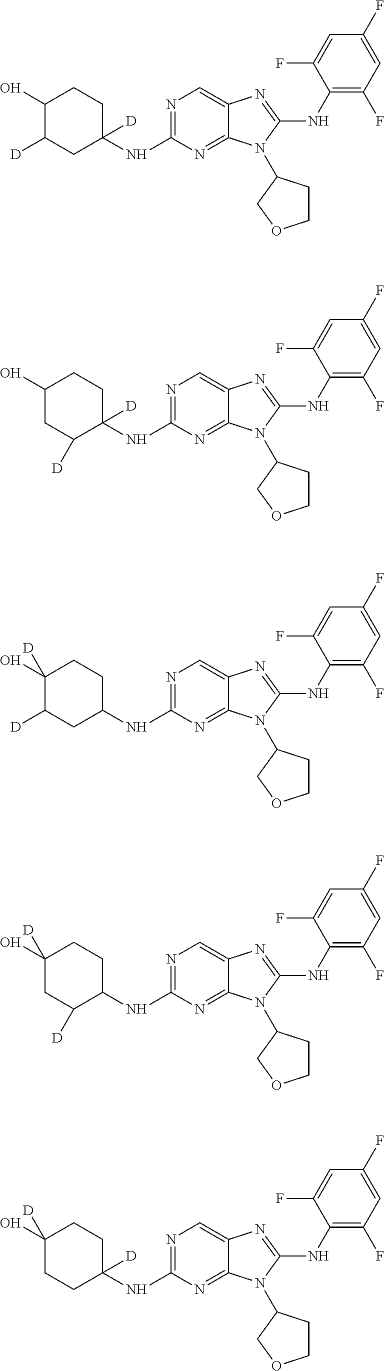 [SMILES: [2H]C1CC([2H])(Nc2ncc3nc(Nc4c(F)cc(F)cc4F)n(C4CCOC4)c3n2)CCC1O ; [2H]C1CC(O)CCC1([2H])Nc1ncc2nc(Nc3c(F)cc(F)cc3F)n(C3CCOC3)c2n1 ; [2H]C1CC(Nc2ncc3nc(Nc4c(F)cc(F)cc4F)n(C4CCOC4)c3n2)CCC1([2H])O ; [2H]C1CC([2H])(O)CCC1Nc1ncc2nc(Nc3c(F)cc(F)cc3F)n(C3CCOC3)c2n1 ; [2H]C1(O)CCC([2H])(Nc2ncc3nc(Nc4c(F)cc(F)cc4F)n(C4CCOC4)c3n2)CC1]

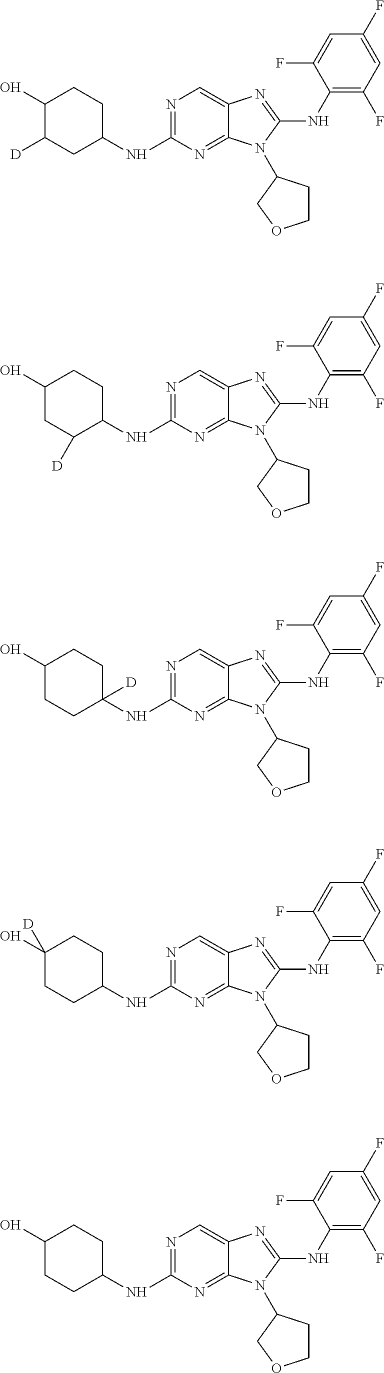 [SMILES: [2H]C1CC(Nc2ncc3nc(Nc4c(F)cc(F)cc4F)n(C4CCOC4)c3n2)CCC1O ; [2H]C1CC(O)CCC1Nc1ncc2nc(Nc3c(F)cc(F)cc3F)n(C3CCOC3)c2n1 ; [2H]C1(Nc2ncc3nc(Nc4c(F)cc(F)cc4F)n(C4CCOC4)c3n2)CCC(O)CC1 ; [2H]C1(O)CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1]

In certain embodiments, one or more Y atoms on the tetrahydrofuranyl portion of Compound 1 are deuterium-enriched. For example, particular compounds provided herein include, but are not limited to, the following listed compounds, wherein the label “D” indicates a deuterium-enriched atomic position, i.e., a sample comprising the given compound has a deuterium enrichment at the indicated position(s) above the natural abundance of deuterium:

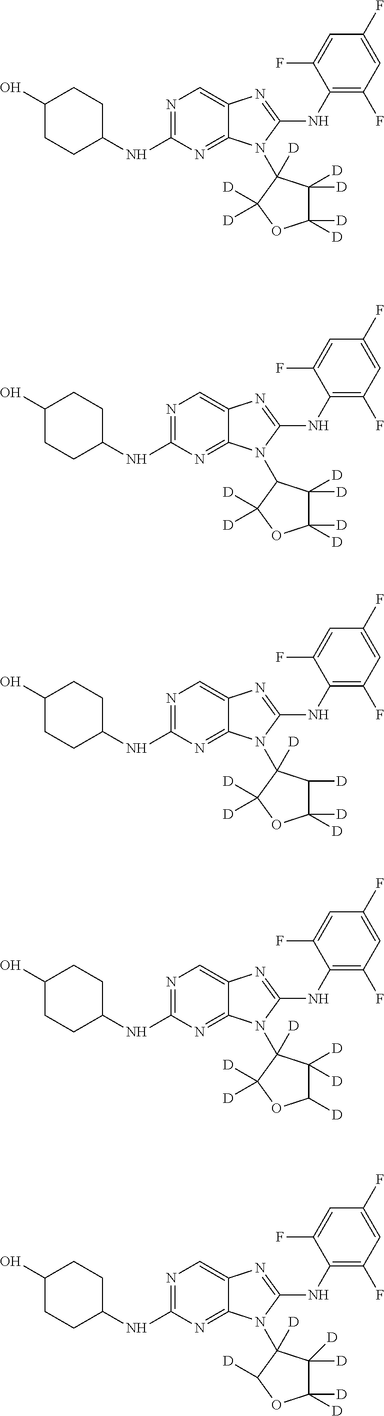 [SMILES: [2H]C1([2H])OC([2H])([2H])C([2H])(n2c(Nc3c(F)cc(F)cc3F)nc3cnc(NC4CCC(O)CC4)nc32)C1([2H])[2H] ; [2H]C1([2H])OC([2H])([2H])C([2H])([2H])C1n1c(Nc2c(F)cc(F)cc2F)nc2cnc(NC3CCC(O)CC3)nc21 ; [2H]C1C([2H])([2H])OC([2H])([2H])C1([2H])n1c(Nc2c(F)cc(F)cc2F)nc2cnc(NC3CCC(O)CC3)nc21 ; [2H]C1OC([2H])([2H])C([2H])(n2c(Nc3c(F)cc(F)cc3F)nc3cnc(NC4CCC(O)CC4)nc32)C1([2H])[2H] ; [2H]C1OC([2H])([2H])C([2H])([2H])C1([2H])n1c(Nc2c(F)cc(F)cc2F)nc2cnc(NC3CCC(O)CC3)nc21]

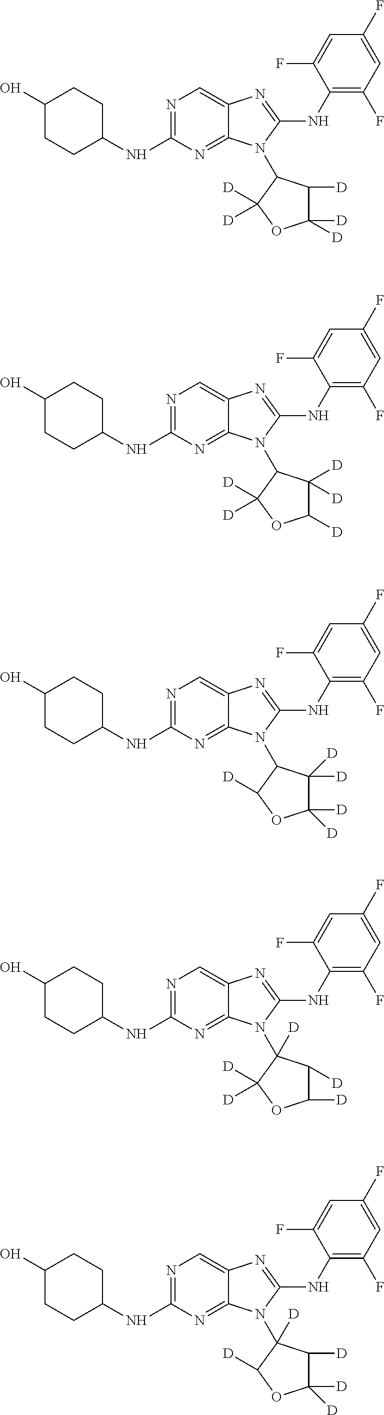 [SMILES: [2H]C1C(n2c(Nc3c(F)cc(F)cc3F)nc3cnc(NC4CCC(O)CC4)nc32)C([2H])([2H])OC1([2H])[2H] ; [2H]C1OC([2H])([2H])C(n2c(Nc3c(F)cc(F)cc3F)nc3cnc(NC4CCC(O)CC4)nc32)C1([2H])[2H] ; [2H]C1OC([2H])([2H])C([2H])([2H])C1n1c(Nc2c(F)cc(F)cc2F)nc2cnc(NC3CCC(O)CC3)nc21 ; [2H]C1OC([2H])([2H])C([2H])(n2c(Nc3c(F)cc(F)cc3F)nc3cnc(NC4CCC(O)CC4)nc32)C1[2H] ; [2H]C1OC([2H])([2H])C([2H])C1([2H])n1c(Nc2c(F)cc(F)cc2F)nc2cnc(NC3CCC(O)CC3)nc21]

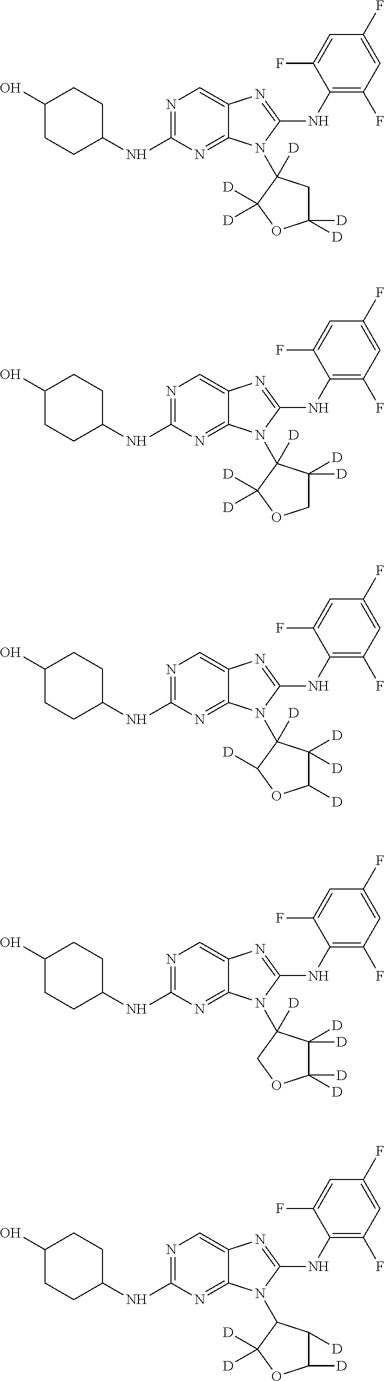 [SMILES: [2H]C1([2H])CC([2H])(n2c(Nc3c(F)cc(F)cc3F)nc3cnc(NC4CCC(O)CC4)nc32)C([2H])([2H])O1 ; [2H]C1([2H])COC([2H])([2H])C1([2H])n1c(Nc2c(F)cc(F)cc2F)nc2cnc(NC3CCC(O)CC3)nc21 ; [2H]C1OC([2H])C([2H])(n2c(Nc3c(F)cc(F)cc3F)nc3cnc(NC4CCC(O)CC4)nc32)C1([2H])[2H] ; [2H]C1([2H])OCC([2H])(n2c(Nc3c(F)cc(F)cc3F)nc3cnc(NC4CCC(O)CC4)nc32)C1([2H])[2H] ; [2H]C1OC([2H])([2H])C(n2c(Nc3c(F)cc(F)cc3F)nc3cnc(NC4CCC(O)CC4)nc32)C1[2H]]

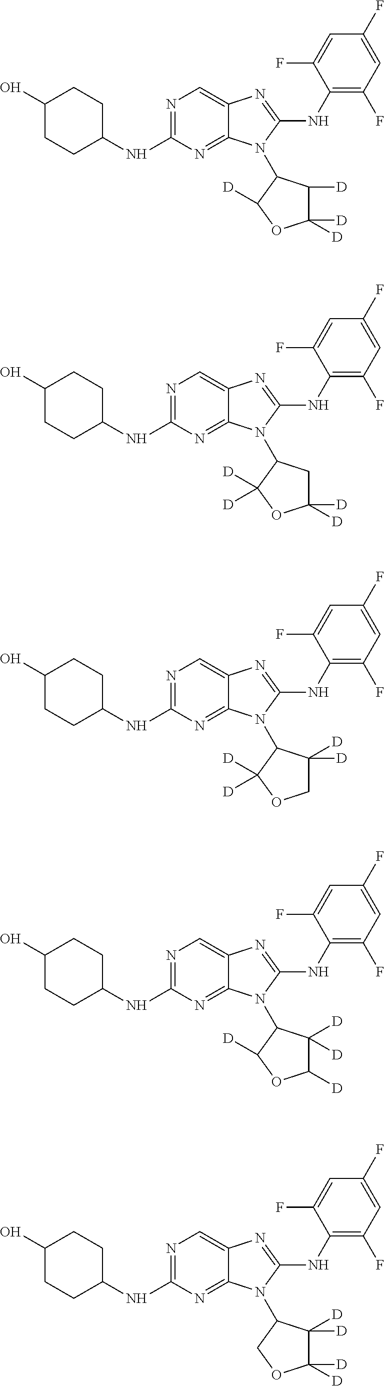 [SMILES: [2H]C1OC([2H])([2H])C([2H])C1n1c(Nc2c(F)cc(F)cc2F)nc2cnc(NC3CCC(O)CC3)nc21 ; [2H]C1([2H])CC(n2c(Nc3c(F)cc(F)cc3F)nc3cnc(NC4CCC(O)CC4)nc32)C([2H])([2H])O1 ; [2H]C1([2H])COC([2H])([2H])C1n1c(Nc2c(F)cc(F)cc2F)nc2cnc(NC3CCC(O)CC3)nc21 ; [2H]C1OC([2H])C([2H])([2H])C1n1c(Nc2c(F)cc(F)cc2F)nc2cnc(NC3CCC(O)CC3)nc21 ; [2H]C1([2H])OCC(n2c(Nc3c(F)cc(F)cc3F)nc3cnc(NC4CCC(O)CC4)nc32)C1([2H])[2H]]

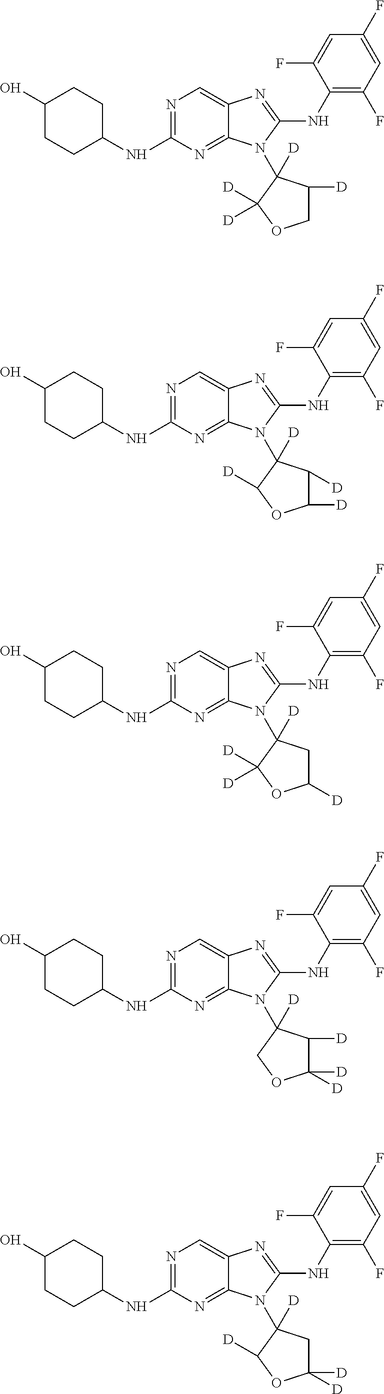 [SMILES: [2H]C1COC([2H])([2H])C1([2H])n1c(Nc2c(F)cc(F)cc2F)nc2cnc(NC3CCC(O)CC3)nc21 ; [2H]C1OC([2H])C([2H])(n2c(Nc3c(F)cc(F)cc3F)nc3cnc(NC4CCC(O)CC4)nc32)C1[2H] ; [2H]C1CC([2H])(n2c(Nc3c(F)cc(F)cc3F)nc3cnc(NC4CCC(O)CC4)nc32)C([2H])([2H])O1 ; [2H]C1C([2H])([2H])OCC1([2H])n1c(Nc2c(F)cc(F)cc2F)nc2cnc(NC3CCC(O)CC3)nc21 ; [2H]C1OC([2H])([2H])CC1([2H])n1c(Nc2c(F)cc(F)cc2F)nc2cnc(NC3CCC(O)CC3)nc21]

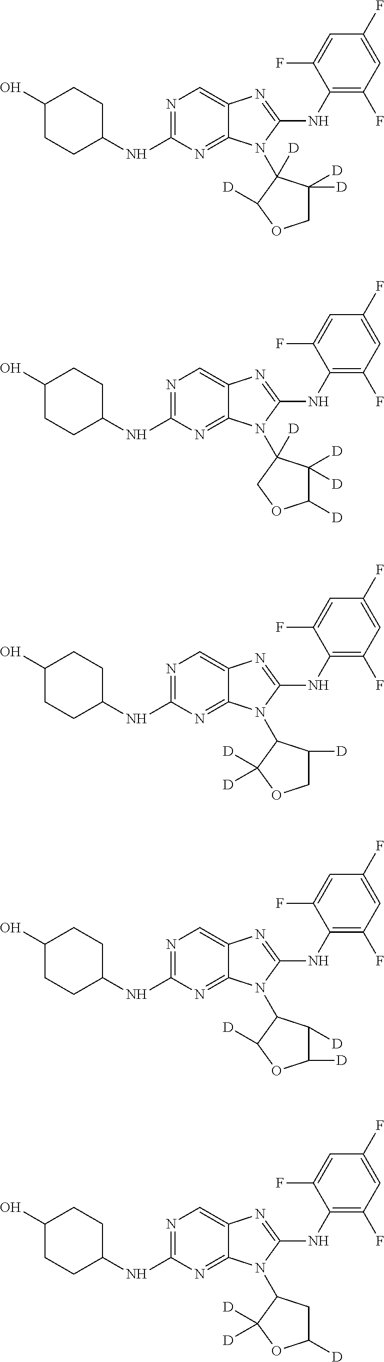 [SMILES: [2H]C1OCC([2H])([2H])C1([2H])n1c(Nc2c(F)cc(F)cc2F)nc2cnc(NC3CCC(O)CC3)nc21 ; [2H]C1OCC([2H])(n2c(Nc3c(F)cc(F)cc3F)nc3cnc(NC4CCC(O)CC4)nc32)C1([2H])[2H] ; [2H]C1COC([2H])([2H])C1n1c(Nc2c(F)cc(F)cc2F)nc2cnc(NC3CCC(O)CC3)nc21 ; [2H]C1OC([2H])C(n2c(Nc3c(F)cc(F)cc3F)nc3cnc(NC4CCC(O)CC4)nc32)C1[2H] ; [2H]C1CC(n2c(Nc3c(F)cc(F)cc3F)nc3cnc(NC4CCC(O)CC4)nc32)C([2H])([2H])O1]

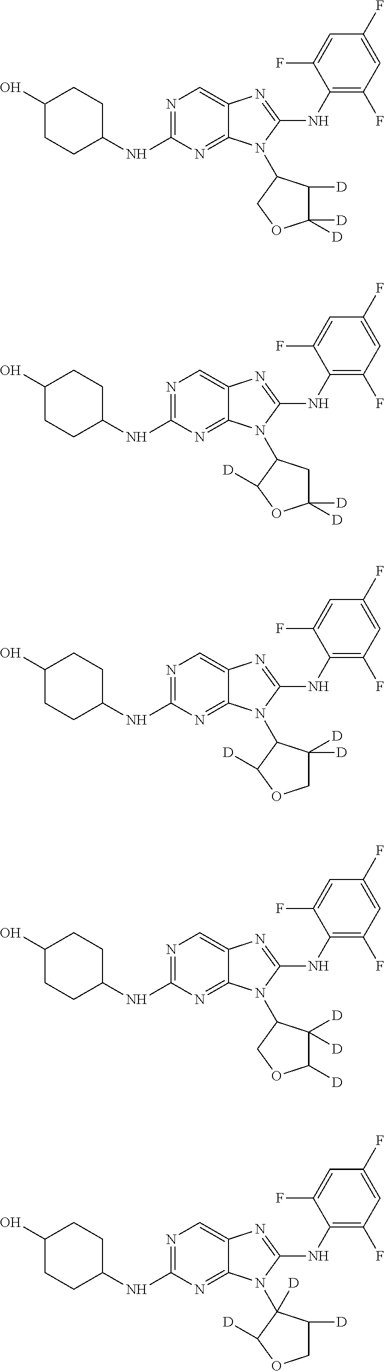 [SMILES: [2H]C1C(n2c(Nc3c(F)cc(F)cc3F)nc3cnc(NC4CCC(O)CC4)nc32)COC1([2H])[2H] ; [2H]C1OC([2H])([2H])CC1n1c(Nc2c(F)cc(F)cc2F)nc2cnc(NC3CCC(O)CC3)nc21 ; [2H]C1OCC([2H])([2H])C1n1c(Nc2c(F)cc(F)cc2F)nc2cnc(NC3CCC(O)CC3)nc21 ; [2H]C1OCC(n2c(Nc3c(F)cc(F)cc3F)nc3cnc(NC4CCC(O)CC4)nc32)C1([2H])[2H] ; [2H]C1COC([2H])C1([2H])n1c(Nc2c(F)cc(F)cc2F)nc2cnc(NC3CCC(O)CC3)nc21]

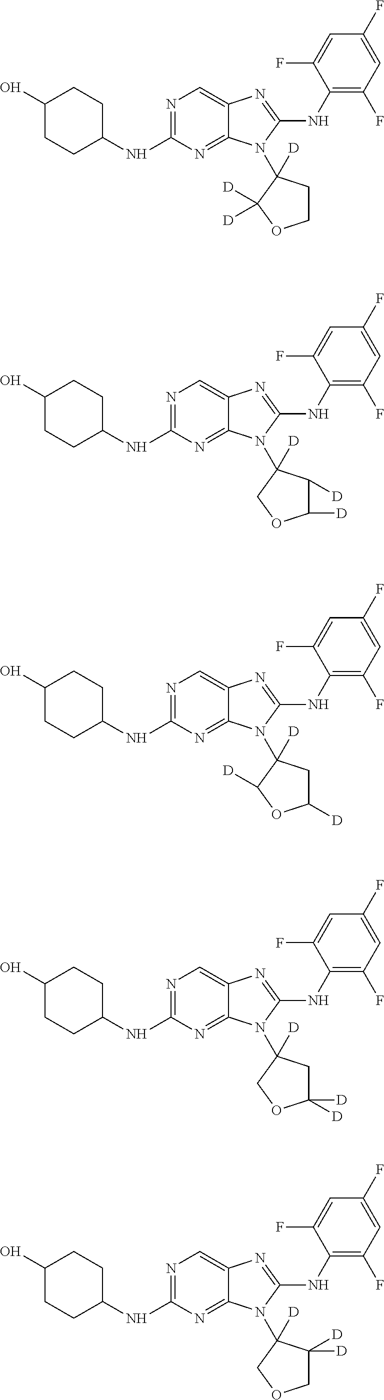 [SMILES: [2H]C1([2H])OCCC1([2H])n1c(Nc2c(F)cc(F)cc2F)nc2cnc(NC3CCC(O)CC3)nc21 ; [2H]C1OCC([2H])(n2c(Nc3c(F)cc(F)cc3F)nc3cnc(NC4CCC(O)CC4)nc32)C1[2H] ; [2H]C1CC([2H])(n2c(Nc3c(F)cc(F)cc3F)nc3cnc(NC4CCC(O)CC4)nc32)C([2H])O1 ; [2H]C1([2H])CC([2H])(n2c(Nc3c(F)cc(F)cc3F)nc3cnc(NC4CCC(O)CC4)nc32)CO1 ; [2H]C1([2H])COCC1([2H])n1c(Nc2c(F)cc(F)cc2F)nc2cnc(NC3CCC(O)CC3)nc21]

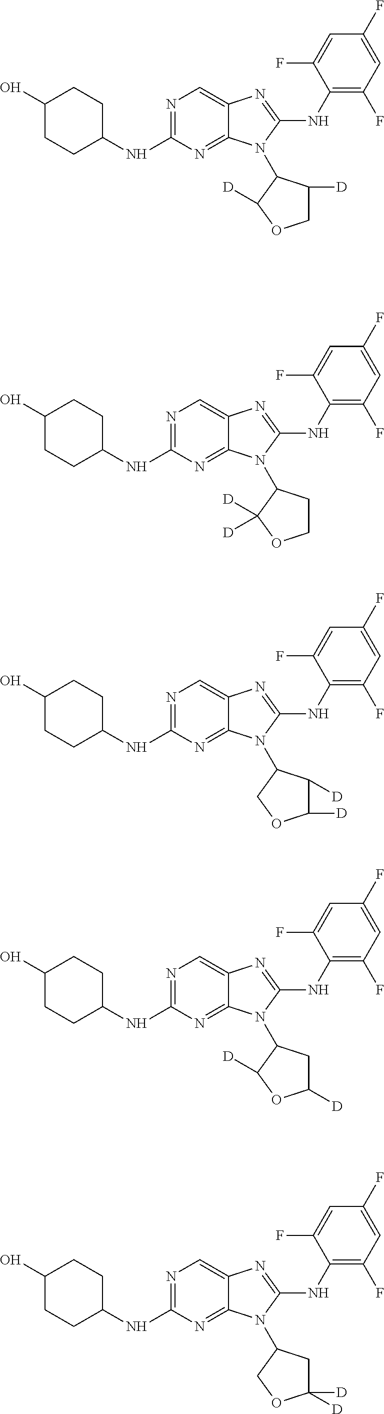 [SMILES: [2H]C1COC([2H])C1n1c(Nc2c(F)cc(F)cc2F)nc2cnc(NC3CCC(O)CC3)nc21 ; [2H]C1([2H])OCCC1n1c(Nc2c(F)cc(F)cc2F)nc2cnc(NC3CCC(O)CC3)nc21 ; [2H]C1OCC(n2c(Nc3c(F)cc(F)cc3F)nc3cnc(NC4CCC(O)CC4)nc32)C1[2H] ; [2H]C1CC(n2c(Nc3c(F)cc(F)cc3F)nc3cnc(NC4CCC(O)CC4)nc32)C([2H])O1 ; [2H]C1([2H])CC(n2c(Nc3c(F)cc(F)cc3F)nc3cnc(NC4CCC(O)CC4)nc32)CO1]

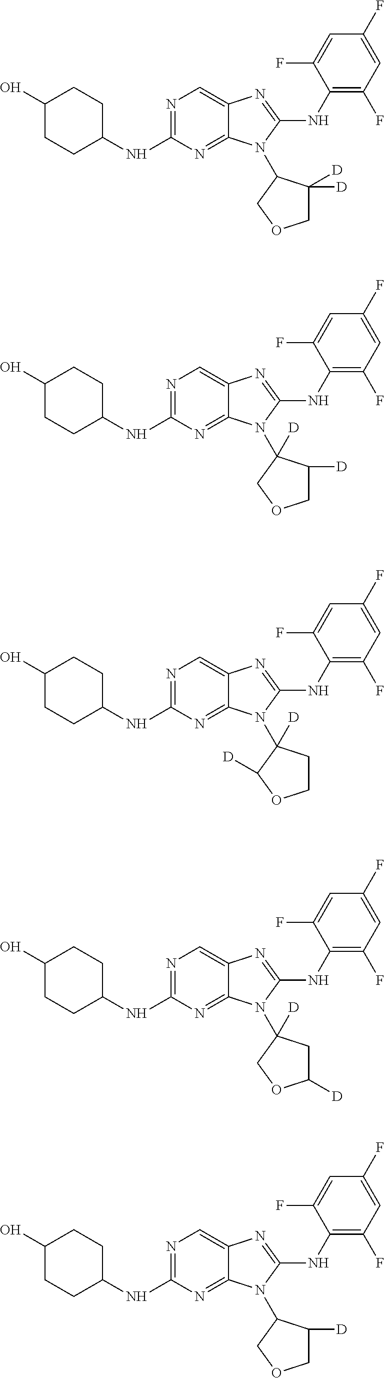 [SMILES: [2H]C1([2H])COCC1n1c(Nc2c(F)cc(F)cc2F)nc2cnc(NC3CCC(O)CC3)nc21 ; [2H]C1COCC1([2H])n1c(Nc2c(F)cc(F)cc2F)nc2cnc(NC3CCC(O)CC3)nc21 ; [2H]C1OCCC1([2H])n1c(Nc2c(F)cc(F)cc2F)nc2cnc(NC3CCC(O)CC3)nc21 ; [2H]C1CC([2H])(n2c(Nc3c(F)cc(F)cc3F)nc3cnc(NC4CCC(O)CC4)nc32)CO1 ; [2H]C1COCC1n1c(Nc2c(F)cc(F)cc2F)nc2cnc(NC3CCC(O)CC3)nc21]

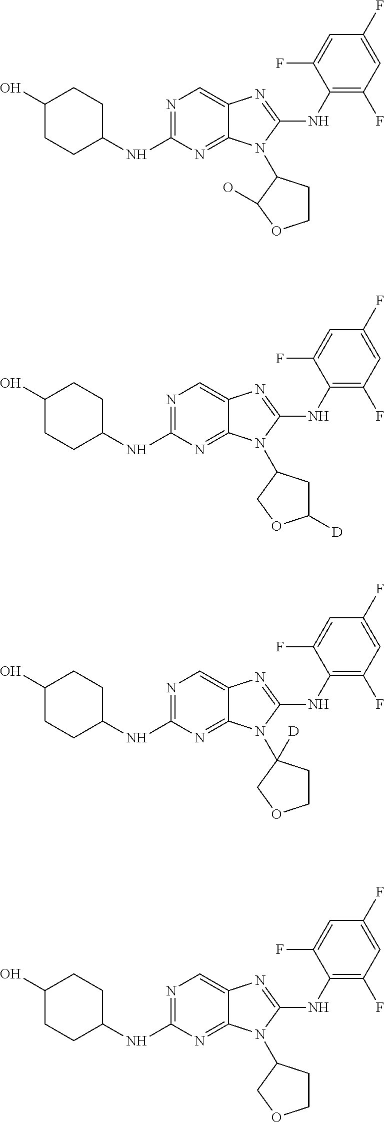 [SMILES: OC1CCC(Nc2ncc3nc(Nc4c(F)cc(F)cc4F)n(C4CCOC4O)c3n2)CC1 ; [2H]C1CC(n2c(Nc3c(F)cc(F)cc3F)nc3cnc(NC4CCC(O)CC4)nc32)CO1 ; [2H]C1(n2c(Nc3c(F)cc(F)cc3F)nc3cnc(NC4CCC(O)CC4)nc32)CCOC1 ; OC1CCC(Nc2ncc3nc(Nc4c(F)cc(F)cc4F)n(C4CCOC4)c3n2)CC1]

In certain embodiments, one or more Y atoms on the phenyl portion of Compound 1 are deuterium-enriched. For example, particular compounds provided herein include, but are not limited to, the following listed compounds, wherein the label “D” indicates a deuterium-enriched atomic position, i.e., a sample comprising the given compound has a deuterium enrichment at the indicated position(s) above the natural abundance of deuterium:

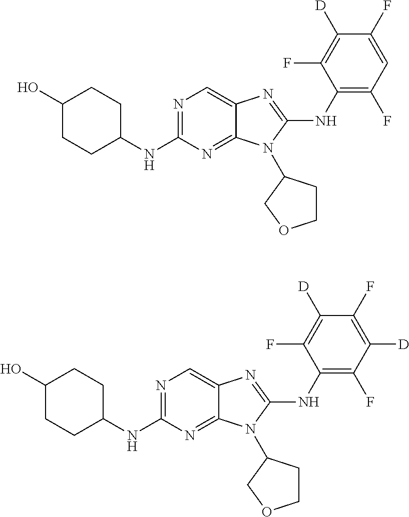 [SMILES: [2H]c1c(F)cc(F)c(Nc2nc3cnc(NC4CCC(O)CC4)nc3n2C2CCOC2)c1F ; [2H]c1c(F)c([2H])c(F)c(Nc2nc3cnc(NC4CCC(O)CC4)nc3n2C2CCOC2)c1F]

In certain embodiments, the purine portion of Compound 1 is deuterium-enriched. For example, particular compounds provided herein include the following compound, wherein the label “D” indicates a deuterium-enriched atomic position, i.e., a sample comprising the given compound has a deuterium enrichment at the indicated position above the natural abundance of deuterium:

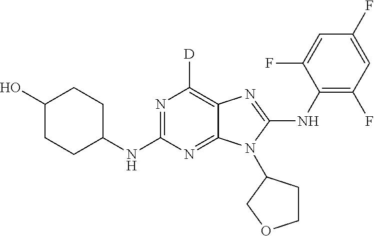 [SMILES: [2H]c1nc(NC2CCC(O)CC2)nc2c1nc(Nc1c(F)cc(F)cc1F)n2C1CCOC1]

In certain embodiments, one or more Y atoms on the cyclohexyl, tetrahydrofuranyl, phenyl and/or purine portions of Compound 1 are deuterium-enriched, i.e., any combination of deuteration enrichment shown above is encompassed. In some embodiments the compound is selected from:

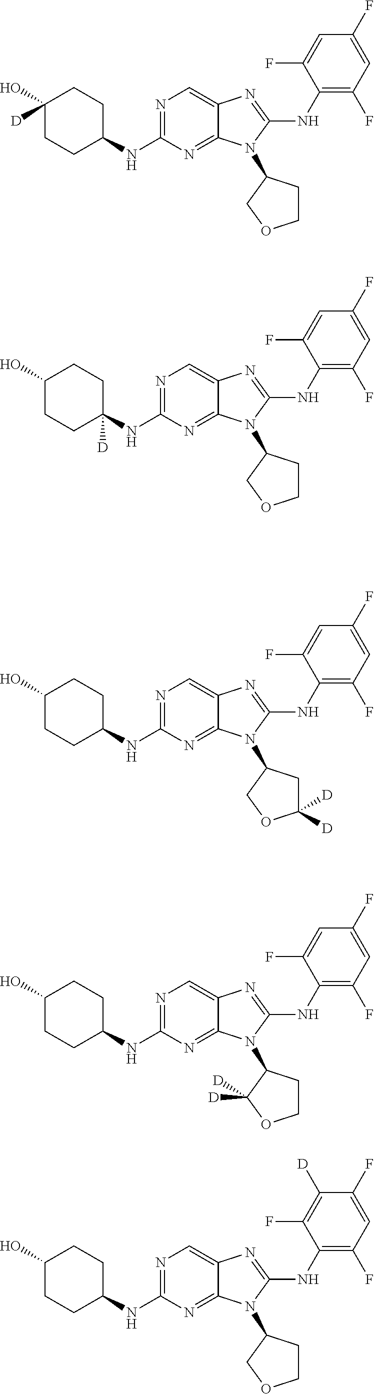

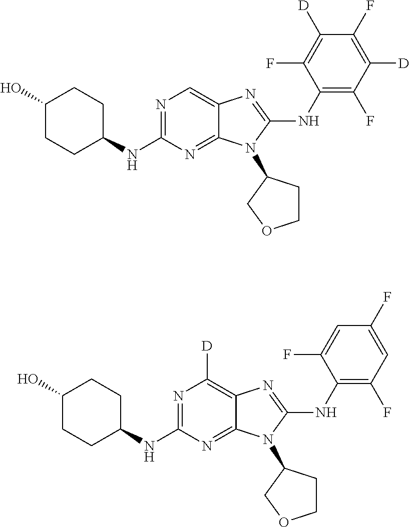 [SMILES: [2H]c1c(F)c([2H])c(F)c(Nc2nc3cnc(N[C@H]4CC[C@H](O)CC4)nc3n2[C@H]2CCOC2)c1F ; [2H]c1nc(N[C@H]2CC[C@H](O)CC2)nc2c1nc(Nc1c(F)cc(F)cc1F)n2[C@H]1CCOC1]

It is understood that one or more deuteriums may exchange with hydrogen under physiological conditions.

In some embodiments, provided herein are carbon-13 analogues of Compound 1, wherein one or more atomic positions of Compound 1 is isotopically enriched with carbon-13. In certain embodiments, provided herein are compounds of the following chemical structure:

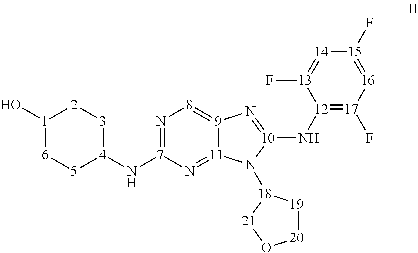

wherein one or more of positions 1, 2, 3, 4, 5, 6, 7, 8, 9, 10, 11, 12, 13, 14, 15, 16, 17, 18, 19, 20 or 21 is/are carbon atom(s) isotopically enriched with carbon-13, and any remaining carbon atom(s) is/are non-enriched carbon atom(s). In particular embodiments, one, two, three, four, five, six, seven, eight, nine, ten, eleven, twelve, thirteen, fourteen, fifteen, sixteen, seventeen, eighteen, nineteen, twenty or twenty-one of carbon atom(s) 1, 2, 3, 4, 5, 6, 7, 8, 9, 10, 11, 12, 13, 14, 15, 16, 17, 18, 19, 20 or 21 is are/isotopically enriched with carbon-13, and any remaining carbon atom(s) is/are non-enriched.

In certain embodiments, one or more carbon atom(s) of the purine portion of compound II, i.e. 7, 8, 9, 10, or 11, is/are carbon-13-enriched. For example, particular compounds provided herein include, but are not limited to, the following compounds, wherein the asterisk -*- indicates a carbon-13 enriched atomic position, i.e., a sample comprising the given compound has a carbon-13 enrichment at the indicated position(s) above the natural abundance of carbon-13.

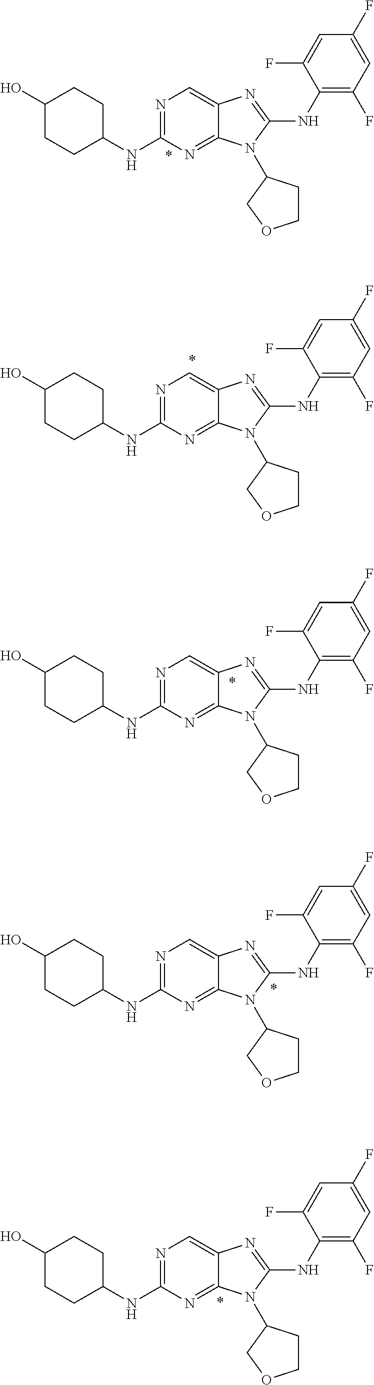 [SMILES: OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1]

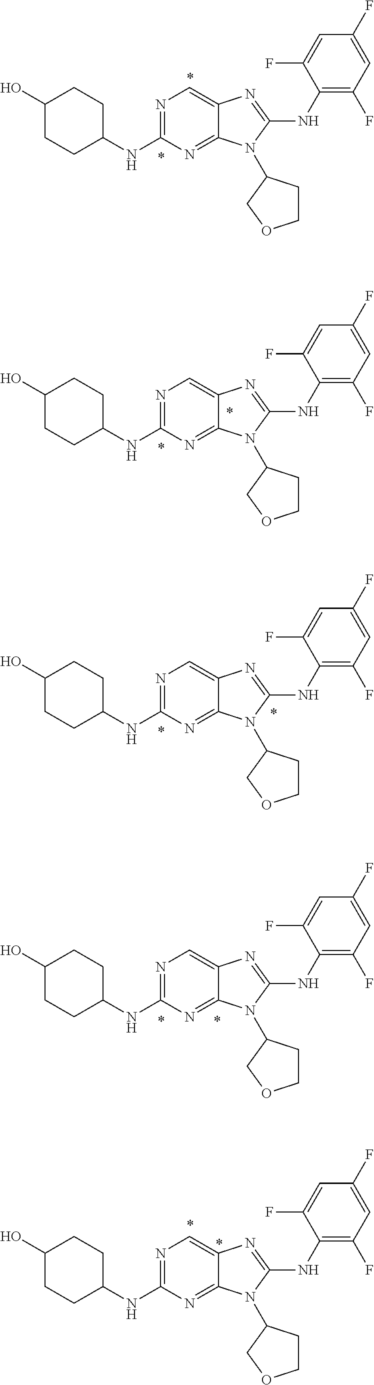 [SMILES: OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1]

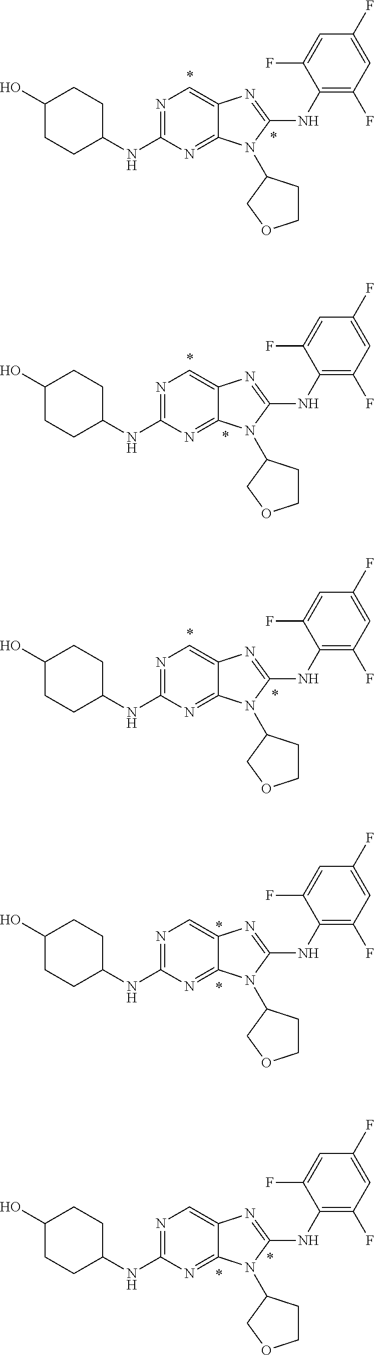 [SMILES: OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1]

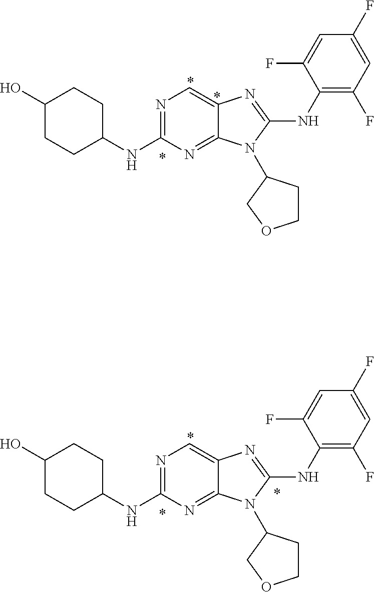 [SMILES: OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1]

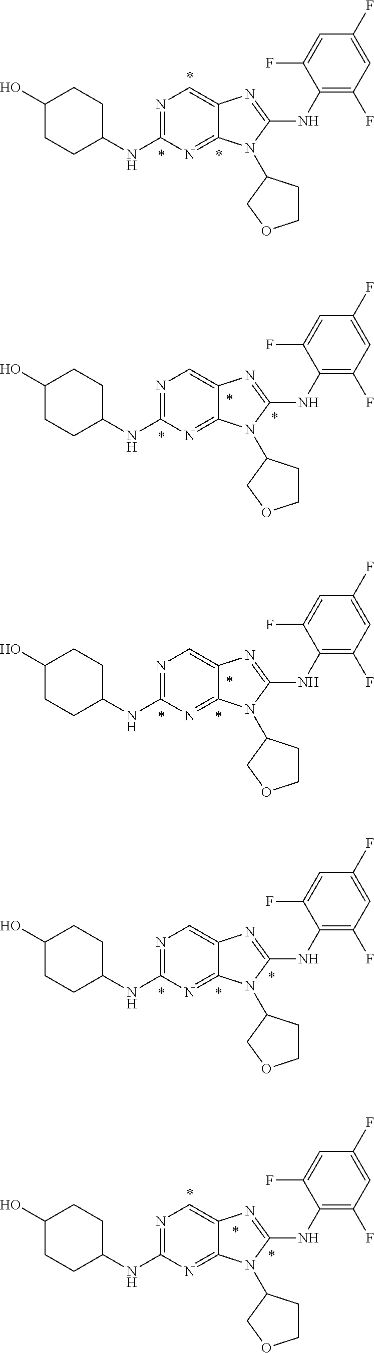 [SMILES: OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1]

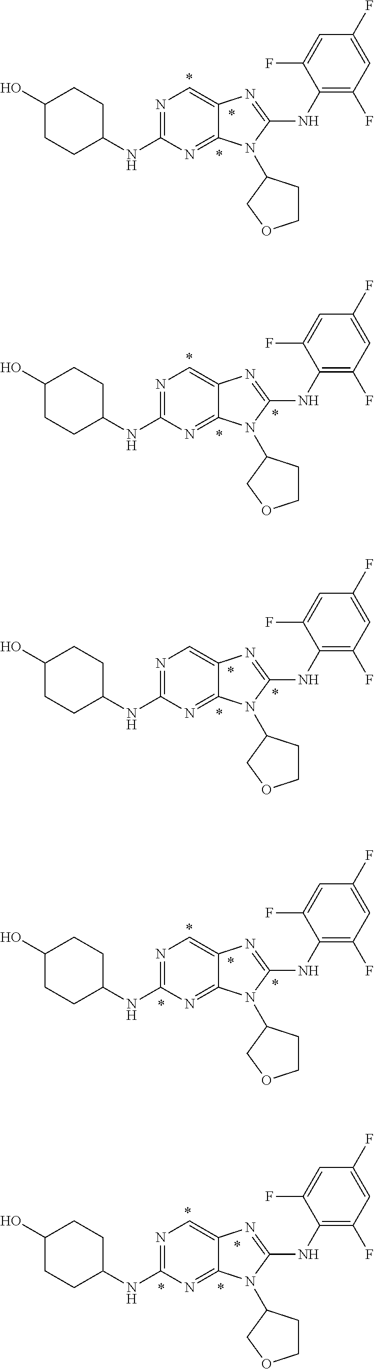 [SMILES: OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1]

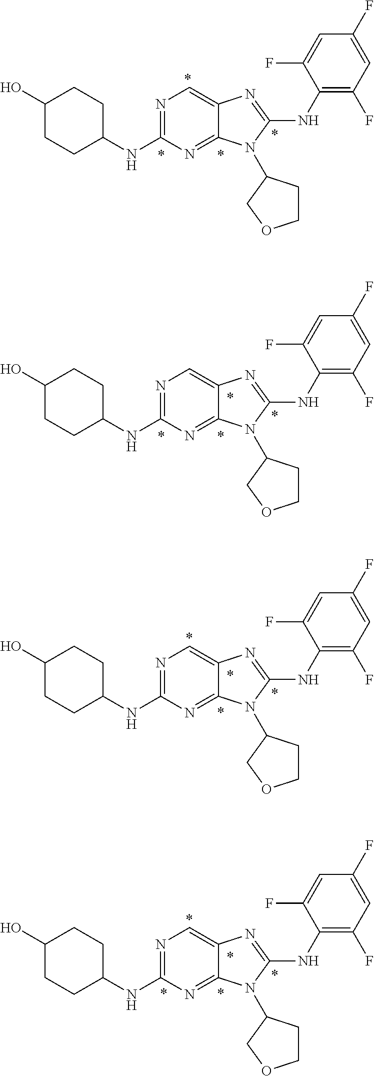 [SMILES: OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1]

In certain embodiments, one or more carbon atom(s) on the cyclohexyl portion of compound II, i.e., 1, 2, 3, 4, 5, or 6, is/are carbon-13-enriched. For example, particular embodiments of compound II provided herein are carbon-13 enriched at the following carbon atoms: 1; 2; 3; 4; 5; 6; 1 and 2; 1 and 3; 1 and 4; 2 and 3; 2 and 4; 2 and 5; 2 and 6; 3 and 4; or 3 and 5. In some embodiments, provided herein are compounds carbon-13 enriched at the following carbon atoms of compound II: 1, 2, and 3; 1, 2, and 4; 1, 2, and 5; 1, 2, and 6; 1, 3, and 4; 1, 3, and 5; 2, 3, and 4; 2, 3, and 5; 2, 3, and 6; 2, 4, and 5; 2, 4, and 6; 2, 5, and 6; or 3, 4, and 5. In some embodiments, provided herein are compounds carbon-13 enriched at the following carbon atoms of compound II: 1, 2, 3, and 4; 1, 2, 3, and 5; 1, 2, 3, and 6; 1, 2, 4, and 5; 1, 2, 4, and 6; 1, 2, 5, and 6; 1, 3, 4, and 5; 2, 3, 4, and 5; 2, 3, 4, and 6; or 2, 3, 5, and 6. In some embodiments, provided herein are compounds carbon-13 enriched at the following carbon atoms of compound II: 1, 2, 3, 4, and 5; 1, 2, 3, 4, and 6; 1, 2, 3, 5, and 6; or 2, 3, 4, 5, and 6. In some embodiments, provided herein are compounds carbon-13 enriched at the following carbon atoms of compound II: 1, 2, 3, 4, 5, and 6.

In certain embodiments, one or more carbon atom(s) on the phenyl portion of compound II, i.e., 12, 13, 14, 15, 16 or 17, is/are carbon-13-enriched. For example, particular embodiments of compound II provided herein are carbon-13 enriched at the following carbon atoms: 12; 13; 14; 15; 16; 17; 12 and 13; 12 and 14; 12 and 15; 13 and 14; 13 and 15; 13 and 16; 13 and 17; 14 and 15; or 14 and 16. In some embodiments, provided herein are compounds carbon-13 enriched at the following carbon atoms of compound II: 12, 13 and 14; 12, 13 and 15; 12, 13 and 16; 12, 13 and 17; 12, 14 and 15; 12, 14 and 16; 13, 14 and 15; 13, 14 and 16; 13, 14 and 17; 13, 15 and 16; 13, 15 and 17; 14, 15 and 16; or 14, 15 and 17. In some embodiments, provided herein are compounds carbon-13 enriched at the following carbon atoms of compound II: 12, 13, 14 and 15; 12, 13, 14, and 16; 12, 13, 14 and 17; 12, 13, 15 and 16; 12, 13, 15 and 17; 12, 14, 15 and 16; 13, 14, 15 and 16; 13, 14, 15 and 17, or 13, 14, 16 and 17. In some embodiments, provided herein are compounds carbon-13 enriched at the following carbon atoms of compound II: 12, 13, 14, 15 and 16; 12, 13, 14, 15 and 17; 12, 13, 14, 16 and 17; or 13, 14, 15, 16 and 17. In some embodiments, provided herein are compounds carbon-13 enriched at the following carbon atoms of compound II: 12, 13, 14, 15, 16 and 17.

In certain embodiments, one or more carbon atom(s) on the tetrahydrofuranyl portion of compound II, i.e., 18, 19, 20, or 21, is/are carbon-13-enriched. For example, particular embodiments of compound II provided herein are carbon-13 enriched at the following carbon atoms: 18; 19; 20; 21; 18 and 19, 18 and 20; 18 and 21; 19 and 20; 19 and 21; or 20 and 21. In some embodiments, provided herein are compounds carbon-13 enriched at the following carbon atoms of compound II: 18, 19 and 20; 18, 19 and 21; 18, 20 and 21; 19, 20 and 21; or 18, 19, 20 and 21.

In certain embodiments, one or more carbon atoms on the purine, cyclohexyl, phenyl and/or tetrahydrofuranyl portions of Compound II is/are carbon-13-enriched, i.e., any combination of isotopically-enriched positions shown above for the purine, cyclohexyl, phenyl and tetrahydrofuranyl portions is encompassed.

In some embodiments, the compound is

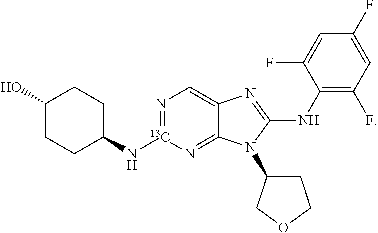 [SMILES: O[C@H]1CC[C@H](N[13c]2ncc3nc(Nc4c(F)cc(F)cc4F)n([C@H]4CCOC4)c3n2)CC1]

In some embodiments, provided herein are nitrogen-15 containing analogues of Compound 1, wherein one or more atomic positions of Compound 1 is isotopically enriched with nitrogen-15. In certain embodiments, provided herein are compounds of the following chemical structure:

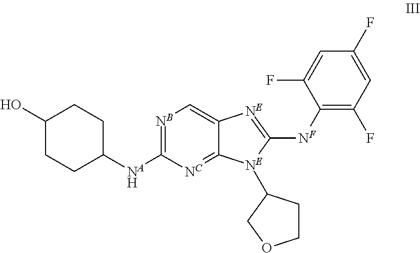

wherein N^{A}, N^{B}, N^{C}, N^{D}, N^{E} or N^{F} is/are isotopically enriched with nitrogen-15, and any remaining nitrogen atom(s) is/are non-enriched nitrogen atom(s). In particular embodiments, one, two, three, or four of N^{A}, N^{B}, N^{C}, N^{D}, N^{E} or N^{F} is are/isotopically enriched with nitrogen-15, and any remaining nitrogen atom(s) is/are non-enriched.

In some embodiments, the compound has one of the following structures, wherein the asterisk -*- indicates a nitrogen-15 enriched atomic position, i.e., a sample comprising the given compound has a nitrogen-15 enrichment at the indicated position(s) above the natural abundance of nitrogen-15.

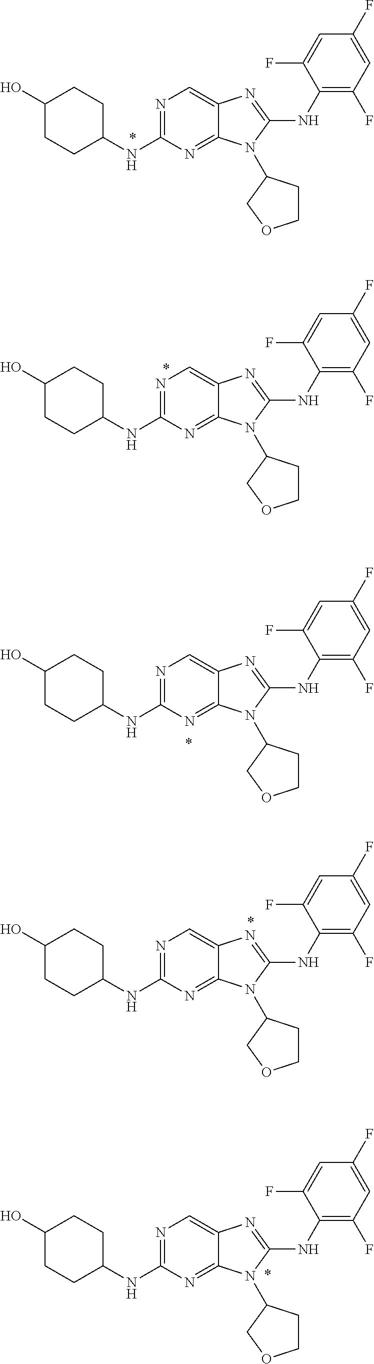 [SMILES: OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1]

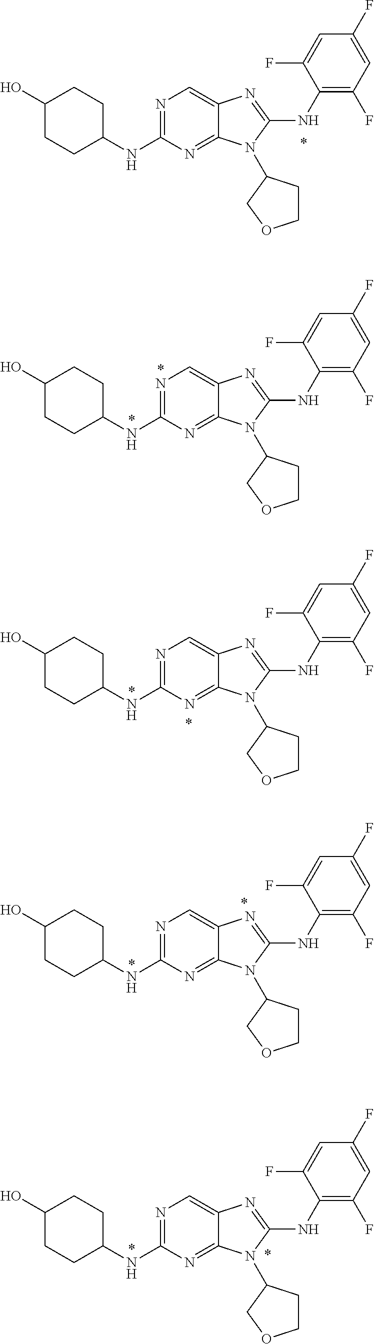 [SMILES: OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1]

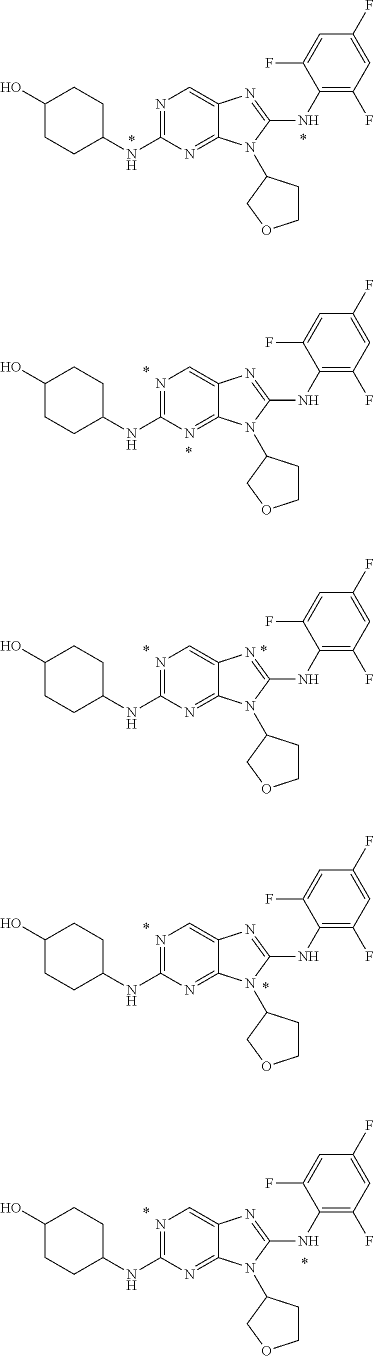 [SMILES: OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1]

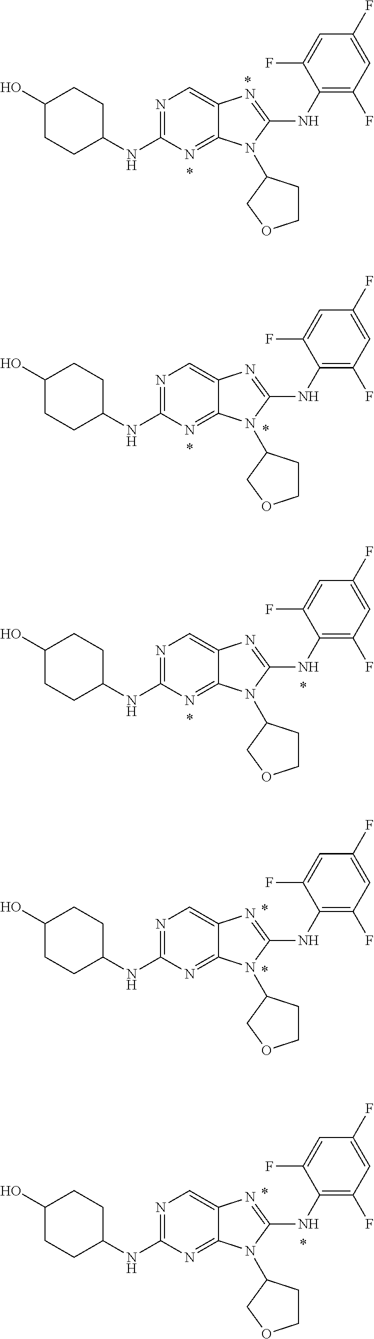 [SMILES: OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1]

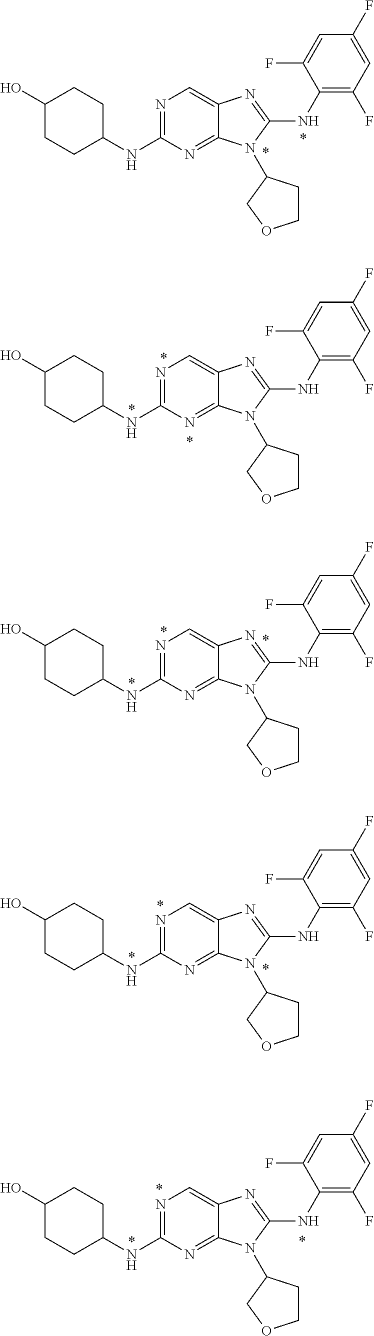 [SMILES: OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1]

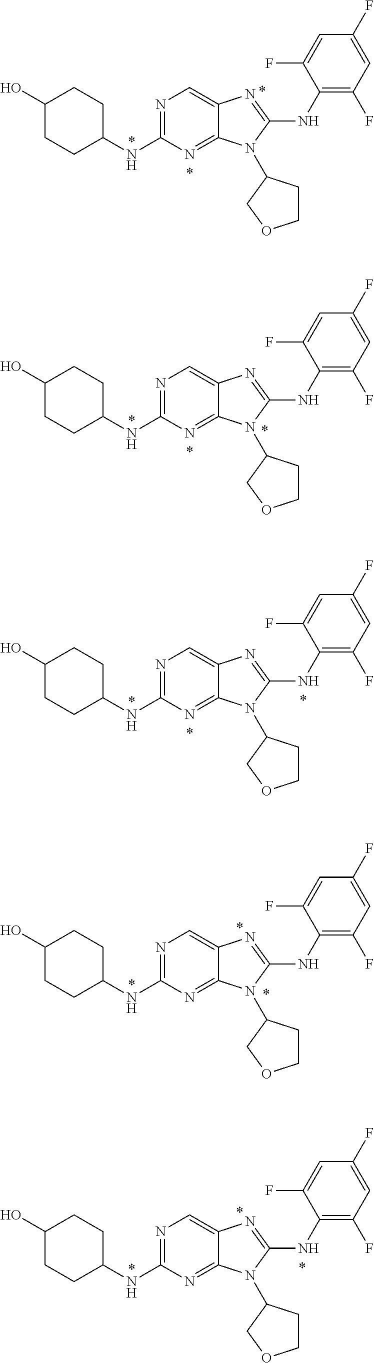 [SMILES: OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1]

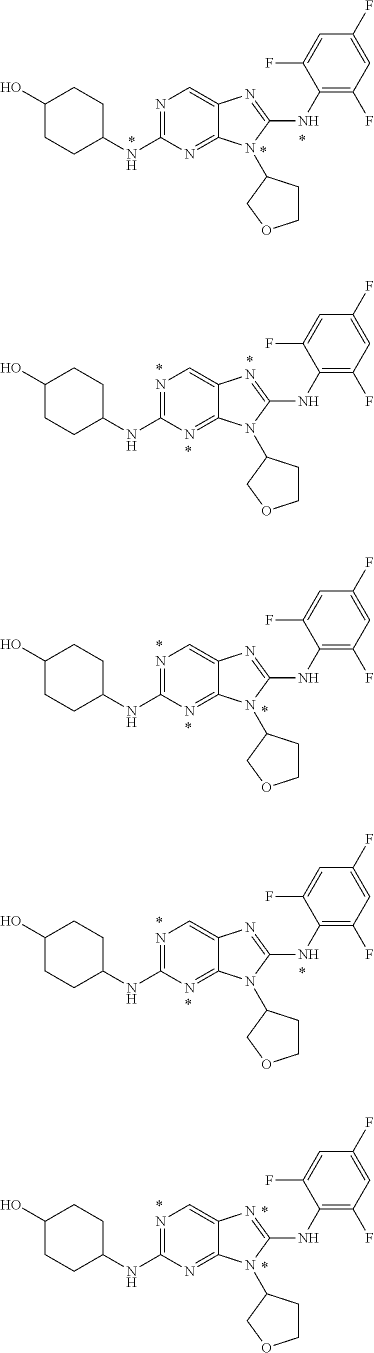 [SMILES: OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1]

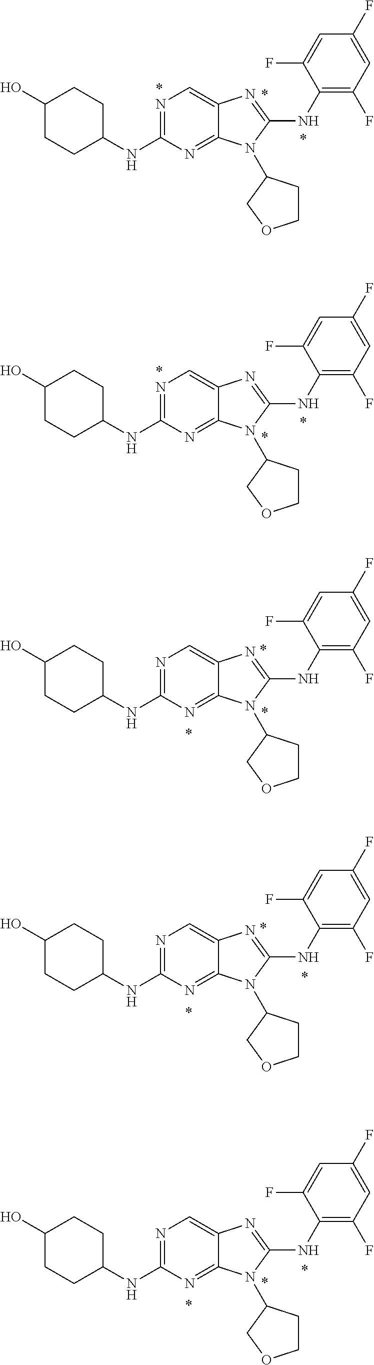 [SMILES: OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1]

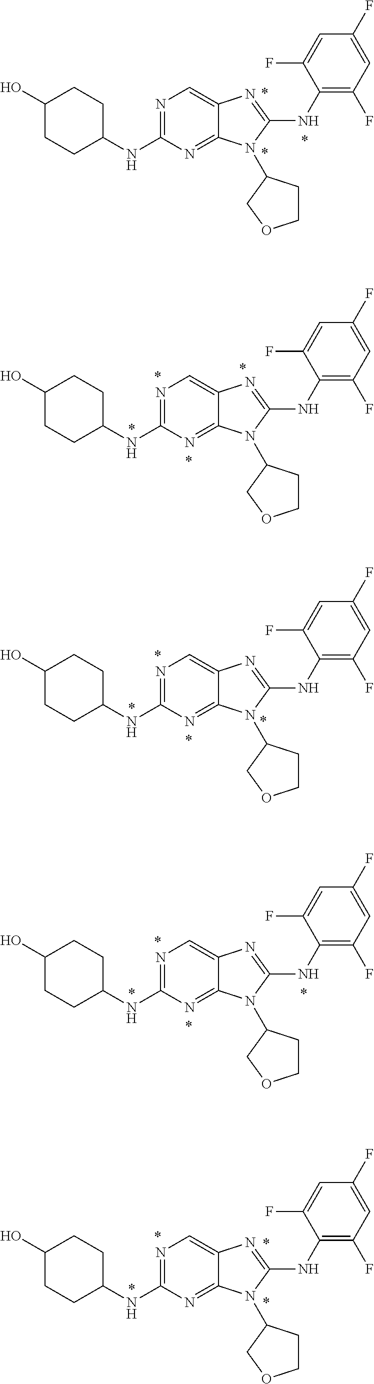 [SMILES: OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1]

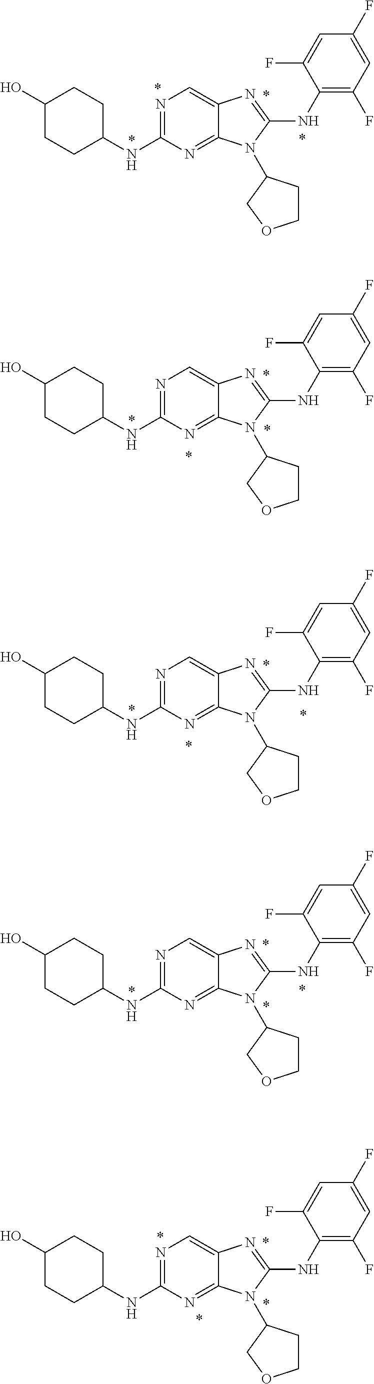 [SMILES: OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1]

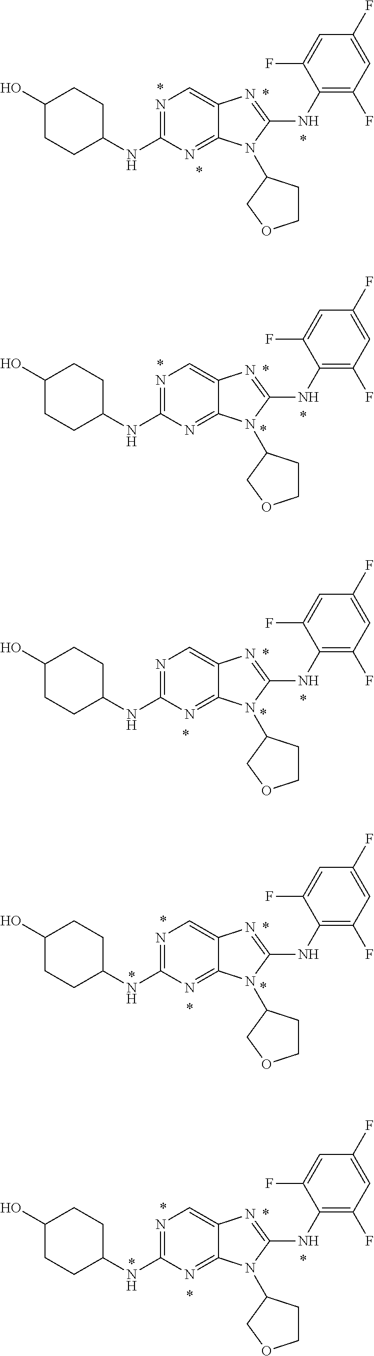 [SMILES: OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1]

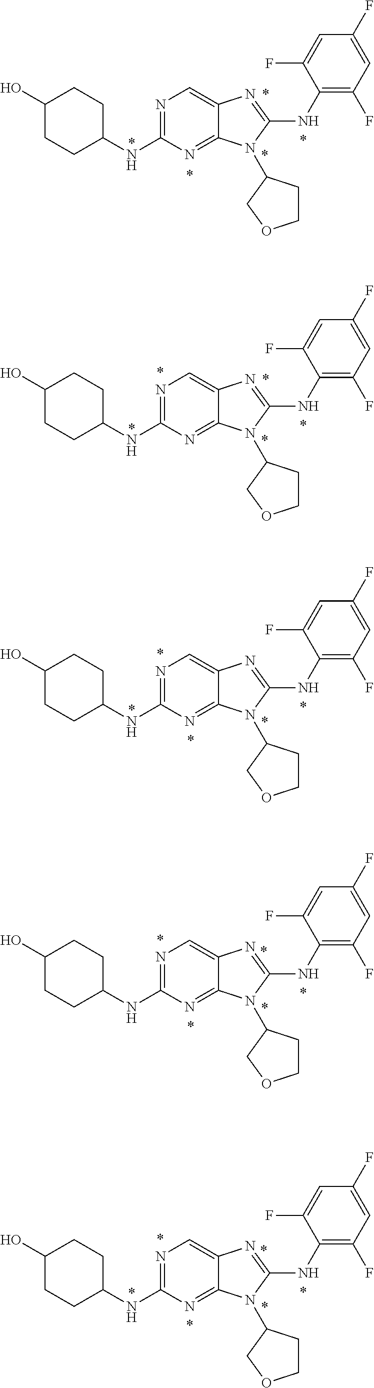 [SMILES: OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1 ; OC1CCC(Nc2ncc3nc(Nc4c(F)cc(F)cc4F)n(C4CCOC4)c3n2)CC1]

In some embodiments, provided herein are oxygen-18 containing analogues of Compound 1, wherein one or more atomic positions of Compound 1 is isotopically enriched with oxygen-18. In certain embodiments, provided herein are compounds of the following chemical structure:

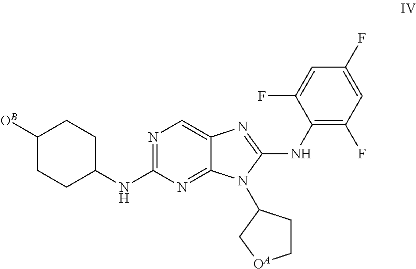

wherein one of O^{A} or O^{B} is/are isotopically enriched with oxygen-18 and the remaining oxygen atom is a non-enriched oxygen atom, or both O^{A} and O^{B} are isotopically enriched with oxygen-18. In one embodiment, O^{A} is isotopically enriched with oxygen-18.

In certain embodiments, one or more hydrogen(s) is/are enriched with deuterium(s) and one or more carbon(s) is/are enriched with carbon-13. In certain embodiments, one or more hydrogen(s) is/are enriched with deuterium and one or more nitrogen(s) is/are enriched with nitrogen-15. In certain embodiments, one or more carbon atom(s) is/are enriched with carbon-13 and one or more nitrogen(s) is/are enriched with nitrogen-15. In certain embodiments, one or more hydrogen(s) is/are enriched with deuterium, one or more carbon(s) are enriched with carbon-13, and one or more nitrogen(s) is/are replaced with nitrogen-15. In some such embodiments, the compound is

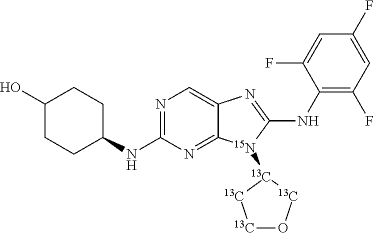

### 4.2. Synthesis

The compounds described herein may be synthesized using methods known to those of ordinary skill in the art. For example, particular compounds described herein are synthesized using standard synthetic organic chemistry techniques known to those of ordinary skill in the art.

In some embodiments, known procedures for the synthesis of Compound 1 are employed, wherein one or more of the reagents, starting materials, precursors, or intermediates are replaced by one or more isotopically-enriched reagents or intermediates, including but not limited to one or more deuterium-enriched reagents, starting materials, precursors, or intermediates, one or more carbon-13-enriched reagents, starting materials, precursors, or intermediates, and/or one or more nitrogen-15-enriched reagents, starting materials, precursors, or intermediates. Such known procedures for the synthesis of Compound 1 include, but are not limited to, those described in U.S. patent application Ser. No. 11/332,617, filed Jan. 12, 2006, International Pub. No. WO 2006/076595, U.S. patent application Ser. No. 11/977,759, filed Oct. 26, 2007, International Pub. No. WO 2008/057252, which are incorporated herein by reference in their entireties. Isotopically enriched reagents, starting materials, precursors, and intermediates are commercially available or may be prepared by routine chemical reactions known to one of skill in the art.

U.S. application Ser. No. 11/332,617 described procedures for synthesizing Compound 1 as shown in the schemes 1 and 2 below.

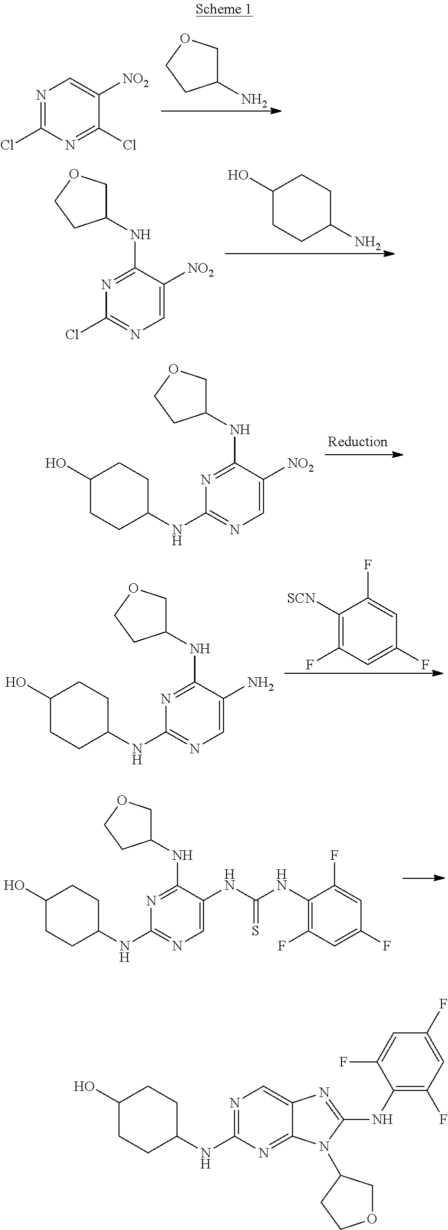

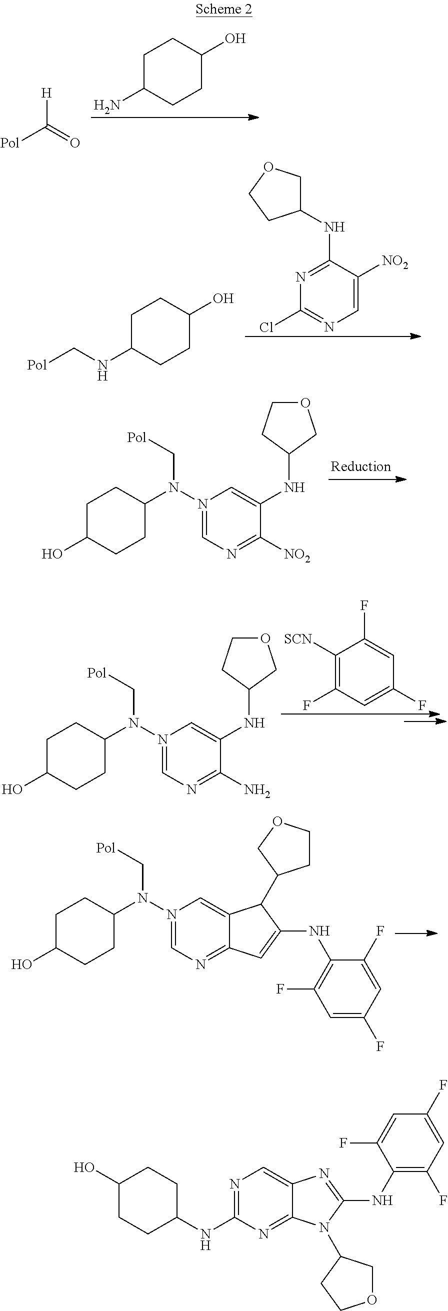

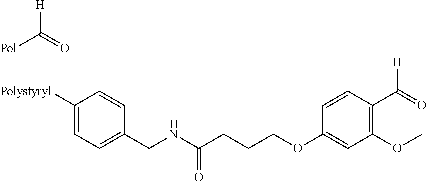

In some embodiments, one or more hydrogen positions of the cyclohexyl, tetrahydrofuranyl, and/or phenyl portion of Compound 1 are enriched with deuterium through organic synthesis. In some embodiments, the methods of Scheme 1 are employed. In particular embodiments, the methods of Scheme 1 are employed, wherein one or more deuterium-enriched precursors are used, as shown in scheme 3 below.

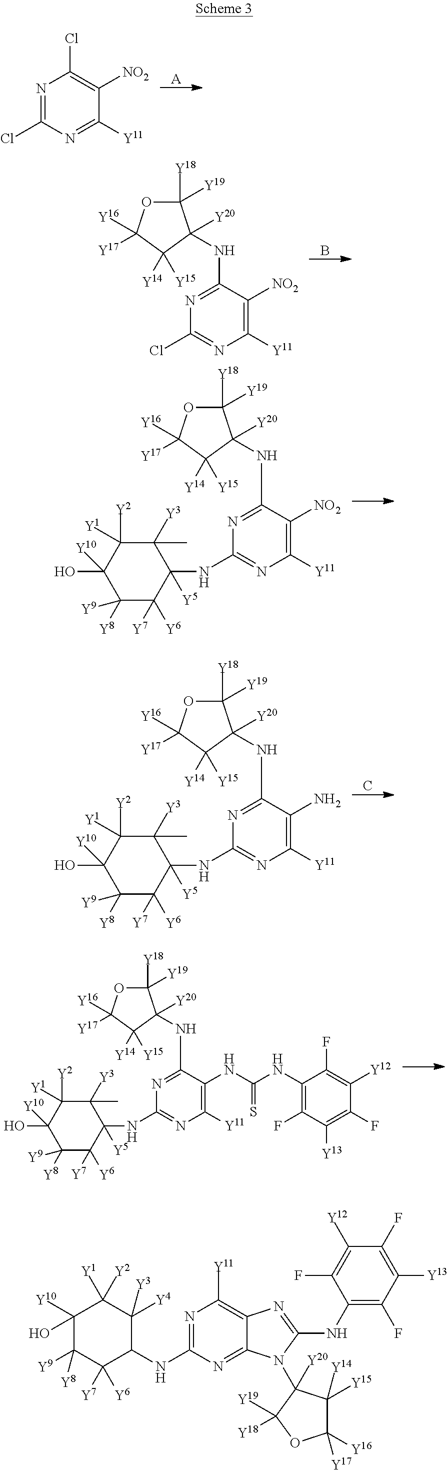

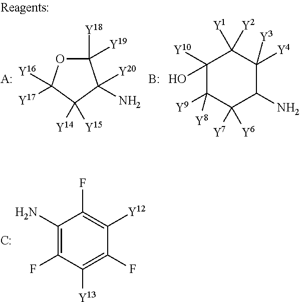

wherein one or more Y atoms (i.e., Y¹⁻²⁰) is/are hydrogen(s) isotopically enriched with deuterium, and any remaining Y atom(s) is/are non-enriched hydrogen atom(s). In particular embodiments, one, two, three, or four of the indicated Y atoms is/are isotopically enriched with deuterium, and any remaining Y atom(s) is/are non-enriched hydrogen(s). Isotopically enriched reagents, starting materials, and/or precursors may be obtained commercially or through techniques known to those of skill in the art.

In some embodiments, the methods described in Scheme 2 are employed. In particular embodiments, the methods of Scheme 2 are employed, wherein deuterium enriched reagents A, B, and/or C are used, similar to above.

In some embodiments, one or more hydrogen positions of the phenyl (and/or purine?) portion are enriched with deuterium through organic synthesis. In certain embodiments, Compound 1 is subjected to reaction conditions suitable for the deuteration of the aromatic ring as shown in the following scheme.

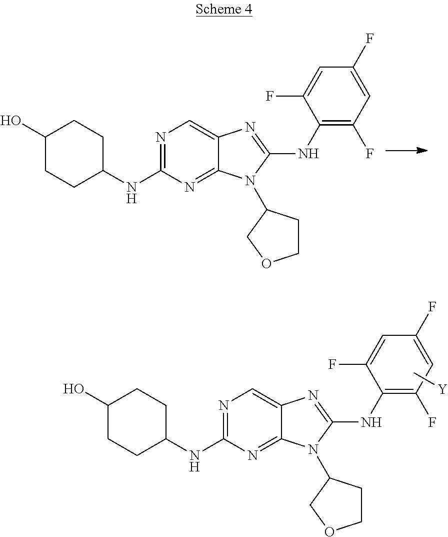

Such conditions are known to those of ordinary skill in the art including for example, those disclosed in the following references, each of which are incorporated herein by reference in their entireties: U.S. Publication No. 2007/0255076; March, J. “Advanced Organic Chemistry, Reactions, Mechanisms, and Structure,” Fourth Ed., Wiley, New York, 1992; Larsen et al., J. Org. Chem., 43(18), 3602, 1978; Blake et al., J. Chem. Soc., Chem. Commun., 930, 1975; and references cited therein. In certain embodiments, Compound 1 is converted to a Compound 1 derivative (e.g. by incorporation of a protecting group), subjected to aromatic deuteration conditions, and converted to deuterium-enriched Compound 1.

In certain embodiments, one or more carbon atoms of Compound 1 are enriched with ¹³C through organic synthesis. In particular embodiments, the methods of Scheme 1 or 2 are employed, wherein a carbon-13 enriched reagent(s) and/or starting material(s) is/are used in the synthesis, as shown in the scheme below.

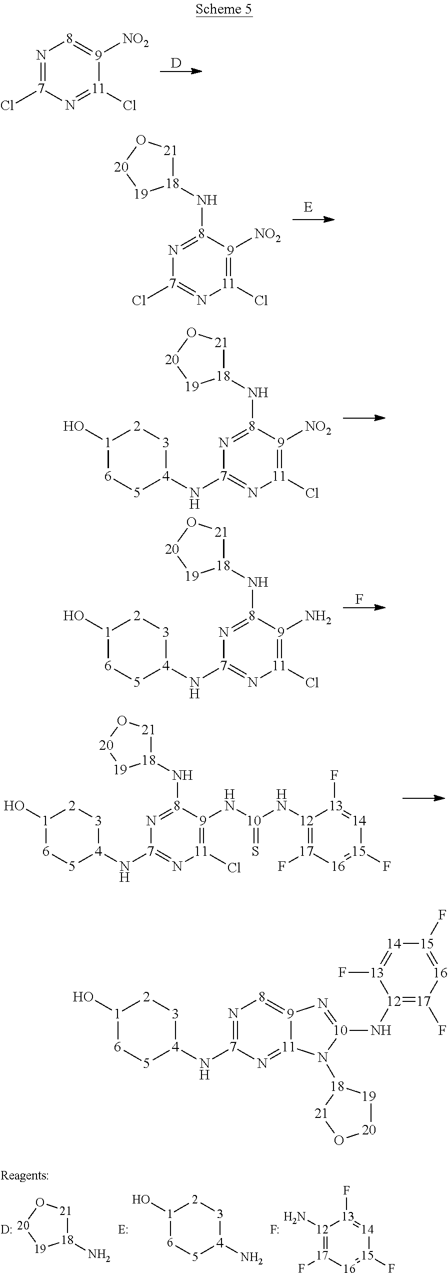

wherein one or more of carbon atoms 1-21 is/are carbon atom(s) that is/are isotopically enriched with carbon-13, and any remaining carbon atom(s) is/are non-enriched carbon atom(s). In particular embodiments, one, two, three, four, or five carbon atom(s) is/are isotopically enriched with carbon-13, and any remaining carbon atom(s) is/are non-enriched carbon atom(s). Carbon enriched reagents and/or starting materials, including Reagents D, E and/or F, may be obtained commercially or through techniques known to those of skill in the art.

In some embodiments, one or more nitrogen atoms of Compound 1 are enriched with nitrogen-15 though organic synthesis. In particular embodiments, the methods of Scheme 1 or 2 are employed, wherein one or more nitrogen-15-enriched reagent(s) and/or starting material(s) is/are used, as shown in the scheme below.

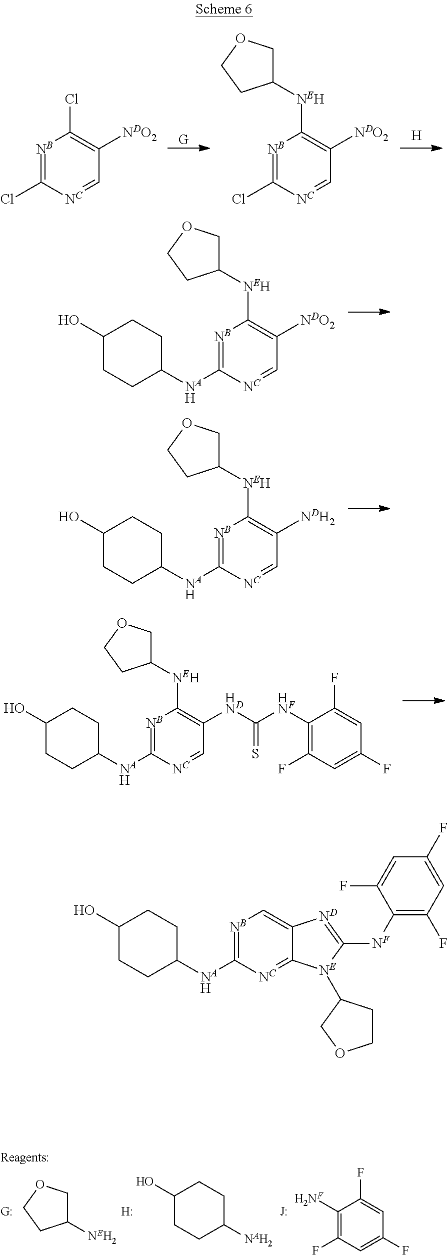

wherein one or more of nitrogen atoms 1-21 is/are nitrogen atom(s) that is/are isotopically enriched with nitrogen-15, and any remaining nitrogen atom(s) is/are non-enriched nitrogen atom(s). In particular embodiments, one, two, three, four, or five nitrogen atom(s) is/are isotopically enriched with nitrogen-15, and any remaining nitrogen atom(s) is/are non-enriched nitrogen atom(s). Nitrogen enriched reagents and/or starting materials, including Reagents G, H and/or J, may be obtained commercially or through techniques known to those of skill in the art.

The routes and methods described above can be modified to provide isotopologues of Compound 1 having both deuterium enrichment and carbon-13 enrichment; both deuterium enrichment and nitrogen-15 enrichment; both carbon-13 enrichment and nitrogen-15 enrichment; or deuterium enrichment, carbon-13 enrichment, and nitrogen-15 enrichment. In some embodiments, the compound is [2-¹⁴C]-4-[(S)-(S)-9-(tetrahydrofuran-3-yl)-8-(2,4,6-trifluorophenylamino)-9H-purin-2-ylamino]cyclohexanol or ¹³C₄,¹⁵N-4-[(S)-9-(Tetrahydrofuran-3-yl)-8-(2,4,6-trifluorophenylamino)-9H-purin-2-ylaminocyclohexanol.

### 4.3 Methods of Treatment, Prevention and Management

Provided herein are methods of treating, preventing, and/or managing various diseases or disorders using isotopologues of Compound 1 as provided herein, or a pharmaceutically acceptable salt, solvate (e.g., hydrate), prodrug, clathrate, or stereoisomer thereof. Without being limited by a particular theory, isotopologues of Compound 1 provided herein have utility as pharmaceuticals to heal or prevent disease in animals or humans. Further, the isotopologues of Compound 1 are active against protein kinases, including those involved in cancer, inflammatory conditions, immunological conditions, neurodegenerative diseases, cardiovascular diseases, metabolic conditions, insulin resistance, diabetes, fibrotic diseases, and disorders caused, induced or exacerbated by ozone, cold or exercise. Accordingly, provided herein are many uses of the isotopologues of Compound 1, including the treatment or prevention of those diseases set forth below, as well as those described in U.S. patent application Ser. No. 11/332,617, filed Jan. 12, 2006, and International Pub. No. WO 2006/076595, U.S. patent application Ser. No. 11/411,413, filed Apr. 26, 2006, published as U.S. Pat. App. Pub. No. 2007/0060598 on Mar. 15, 2007, and U.S. patent application Ser. No. 11/708,150, filed Feb. 15, 2007, the entireties of each of which is incorporated by reference herein. The methods provided herein comprise the administration of an effective amount of an isotopologue of Compound 1 to a patient in need thereof.

Representative autoimmune conditions that the isotopologues of Compound 1 are useful for treating or preventing include, but are not limited to, rheumatoid arthritis, rheumatoid spondylitis, osteoarthritis, multiple sclerosis, lupus, inflammatory bowel disease, ulcerative colitis, Crohn's disease, myasthenia gravis, Graves disease and diabetes (e.g., Type I diabetes).

Representative inflammatory conditions that the isotopologues of Compound 1 are useful for treating or preventing include, but are not limited to, asthma and allergic rhinitis, bronchitis, chronic obstructive pulmonary disease, cystic fibrosis, inflammatory bowel disease, irritable bowel syndrome, Crohn's disease, mucous colitis, ulcerative colitis, diabetes (e.g., Type I diabetes and Type II diabetes) and obesity.

Representative metabolic conditions that the isotopologues of Compound 1 are useful for treating or preventing include, but are not limited to, obesity and diabetes (e.g., Type II diabetes). In a particular embodiment, provided herein are methods for the treatment or prevention of insulin resistance. In certain embodiments, provided herein are methods for the treatment or prevention of insulin resistance that leads to diabetes (e.g., Type II diabetes).

In another embodiment, provided herein are methods for the treatment or prevention of syndrome X or metabolic syndrome. In another embodiment, provide herein are methods for the treatment or prevention of diabetes. In another embodiment, provide herein are methods for the treatment or prevention of Type II diabetes, Type I diabetes, slow-onset Type I diabetes, diabetes insipidus (e.g., neurogenic diabetes insipidus, nephrogenic diabetes insipidus, dipsogenic diabetes insipidus, or gestagenic diabetes insipidus), diabetes mellitus, gestational diabetes mellitus, polycystic ovarian syndrome, maturity-onset diabetes, juvenile diabetes, insulin-dependant diabetes, non-insulin dependant diabetes, malnutrition-related diabetes, ketosis-prone diabetes, pre-diabetes (e.g., imparied glucose metabolism), cystic fibrosis related diabetes, hemochromatosis and ketosis-resistant diabetes.

In another embodiment, provided herein are methods for the treatment or prevention of fibrotic diseases and disorders. In a particular embodiment, provided herein are methods for the treatment or prevention of idiopathic pulmonary fibrosis, myelofibrosis, hepatic fibrosis, steatofibrosis and steatohepatitis.

Representative cardiovascular diseases that the isotopologues of Compound 1 are useful for treating or preventing include, but are not limited to, stroke, myocardial infarction or ischemic damage to the heart, lung, gut, kidney, liver, pancreas, spleen or brain.

Representative cardiovascular and renal diseases that an isotopologue of Compound 1 containing or coated stent or stent graft is useful for treating or preventing include atherosclerosis and the treatment or prevention of restenosis after vascular intervention such as angioplasty.

In another embodiment, provided herein are methods for improved processing of beta-islet cells (e.g., human) for transplantation.

In another embodiment, provided herein are methods for improved culturing of beta-islet cells (e.g., human) for transplantation.

In another embodiment, provided herein are methods for improved viability of beta-islet cells (e.g., human) for transplantation.

In another embodiment, provided herein are methods for improved graft-survival of beta-islet cells (e.g., human) for transplantation.

In another embodiment, provided herein are methods for improved processing, culturing, viability and graft-survival of beta-islet cells (e.g., human) for transplantation.

An isotopologue of Compound 1 containing or coated stent or stent graft can further comprise an effective amount of another active agent useful for treating or preventing a cardiovascular or renal disease, including, but are not limited to, an anticoagulant agent, an antimetabolite agent, an anti-inflammatory agent, an antiplatelet agent, an antithrombin agent, an antimitotic agent, a cytostatic agent or an antiproliferative agent.

The isotopologues of Compound 1 are also useful for treating or preventing ischemia/reperfusion injury in general. Accordingly, the isotopologues of Compound 1 are useful for treating or preventing acute or chronic organ transplant rejection and for the preservation of tissue and organs.

Representative cancers that the isotopologues of Compound 1 are useful for treating or preventing include, but are not limited to, cancers of the head, neck, eye, mouth, throat, esophagus, bronchus, larynx, pharynx, chest, bone, lung, colon, rectum, stomach, prostate, urinary bladder, uterine, cervix, breast, ovaries, testicles or other reproductive organs, skin, thyroid, blood, lymph nodes, kidney, liver, pancreas, and brain or central nervous system.

Cancers within the scope of the methods provided herein include those associated with BCR-ABL, and mutants or isoforms thereof, as well as kinases from the src kinase family, kinases from the Rsk kinase family, kinases from the CDK family, kinases from the MAPK kinase family, and tyrosine kinases such as Fes, Lyn, and Syk kinases, and mutants or isoforms thereof.

In a particular embodiment, provided herein are methods for the treatment or prevention of a disease or disorder associated with the modulation, for example inhibition, of a kinase, including, but are not limited to, tyrosine-protein kinase (SYK), tyrosine-protein kinase (ZAP-70), protein tyrosine kinase 2 beta (PYK2), focal adhesion kinase 1 (FAK), B lymphocyte kinase (BLK), hemopoietic cell kinase (HCK), v-yes-1 Yamaguchi sarcoma viral related oncogene homolog (LYN), T cell-specific protein-tyrosine kinase (LCK), proto-oncogene tyrosine-protein kinase (YES), proto-oncogene tyrosine-protein kinase (SRC), proto-oncogene tyrosine-protein kinase (FYN), proto-oncogene tyrosine-protein kinase (FGR), proto-oncogene tyrosine-protein kinase (FER), proto-oncogene tyrosine-protein kinase (FES), C-SRC kinase, protein-tyrosine kinase (CYL), tyrosine protein kinase (CSK), megakaryocyte-associated tyrosine-protein kinase (CTK), tyrosine-protein kinase receptor (EPH), Ephrin type-A receptor 1, Ephrin type-A receptor 4 (EPHA4), Ephrin type-B receptor 3 (EPHB3), Ephrin type-A receptor 8 (EPHA8), neurotrophic tyrosine kinase receptor, type 1 (NTRK1), protein-tyrosine kinase (PTK2), syk-related tyrosine kinase (SRK), protein tyrosine kinase (CTK), tyro3 protein tyrosine kinase (TYRO3), bruton agammaglobulinemia tyrosine kinase (BTK), leukocyte tyrosine kinase (LTK), protein-tyrosine kinase (SYK), protein-tyrosine kinase (STY), tek tyrosine kinase (TEK), elk-related tyrosine kinase (ERK), tyrosine kinase with immunoglobulin and egf factor homology domains (TIE), protein tyrosine kinase (TKF), neurotrophic tyrosine kinase, receptor, type 3 (NTRK3), mixed-lineage protein kinase-3 (MLK3), protein kinase, mitogen-activated 4 (PRKM4), protein kinase, mitogen-activated 1 (PRKM 1), protein tyrosine kinase (PTK7), protein tyrosine kinase (EEK), minibrain (drosophila) homolog (MNBH), bone marrow kinase, x-linked (BMX), eph-like tyrosine kinase 1 (ETK1), macrophage stimulating 1 receptor (MST1R), btk-associated protein, 135 kd, lymphocyte-specific protein tyrosine kinase (LCK), fibroblast growth factor receptor-2 (FGFR2), protein tyrosine kinase-3 (TYK3), protein tyrosine kinase (TXK), tec protein tyrosine kinase (TEC), protein tyrosine kinase-2 (TYK2), eph-related receptor tyrosine kinase ligand 1 (EPLG1), t-cell tyrosine kinase (EMT), eph tyrosine kinase 1 (EPHT1), zona pellucida receptor tyrosine kinase, 95 kd (ZRK), protein kinase, mitogen-activated, kinase 1 (PRKMK1), eph tyrosine kinase 3 (EPHT3), growth arrest-specific gene-6 (GAS6), kinase insert domain receptor (KDR), axl receptor tyrosine kinase (AXL), fibroblast growth factor receptor-1 (FGFR1), v-erb-b2 avian erythroblastic leukemia viral oncogene homolog 2 (ERBB2), fms-like tyrosine kinase-3 (FLT3), neuroepithelial tyrosine kinase (NEP), neurotrophic tyrosine kinase receptor-related 3 (NTRKR3), eph-related receptor tyrosine kinase ligand 5 (EPLG5), neurotrophic tyrosine kinase, receptor, type 2 (NTRK2), receptor-like tyrosine kinase (RYK), tyrosine kinase, b-lymphocyte specific (BLK), eph tyrosine kinase 2 (EPHT2), eph-related receptor tyrosine kinase ligand 2 (EPLG2), glycogen storage disease VIII, eph-related receptor tyrosine kinase ligand 7 (EPLG7), janus kinase 1 (JAK1), fms-related tyrosine kinase-1 (FLT1), protein kinase, camp-dependent, regulatory, type I, alpha (PRKAR1A), wee-1 tyrosine kinase (WEE1), eph-like tyrosine kinase 2 (ETK2), receptor tyrosine kinase musk, insulin receptor (INSR), janus kinase 3 (JAK3), fms-related tyrosine kinase-3 ligand protein kinase c, beta 1 (PRKCB1), tyrosine kinase-type cell surface receptor (HER3), janus kinase 2 (JAK2), lim domain kinase 1 (LIMK1), dual specificity phosphatase 1 (DUSP1), hemopoietic cell kinase (HCK), tyrosine 3-monooxygenase/tryptophan 5-monooxygenase activation protein, eta polypeptide (YWHAH), ret proto-oncogene (RET), tyrosine 3-monooxygenase/tryptophan 5-monooxygenase activation protein, zeta polypeptide (YWHAZ), tyrosine 3-monooxygenase/tryptophan 5-monooxygenase activation protein, beta polypeptide (YWHAB), hepatoma transmembrane kinase (HTK), map kinase kinase 6, phosphatidylinositol 3-kinase, catalytic, alpha polypeptide (PIK3CA), cyclin-dependent kinase inhibitor 3 (CDKN3), diacylglycerol kinase, delta, 130 kd, protein-tyrosine phosphatase, nonreceptor type, 13 (PTPN13), abelson murine leukemia viral oncogene homolog 1 (ABL1), diacylglycerol kinase, alpha (DAGK1), focal adhesion kinase 2, epithelial discoidin domain receptor 1 (EDDR1), anaplastic lymphoma kinase (ALK), phosphatidylinositol 3-kinase, catalytic, gamma polypeptide (PIK3CG), phosphatidylinositol 3-kinase regulatory subunit, (PIK3R1), eph homology kinase-1 (EHK1), v-kit hardy-zuckerman 4 feline sarcoma viral oncogene homolog (KIT), fibroblast growth factor receptor-3 (FGFR3), vascular endothelial growth factor c (VEGFC), epidermal growth factor receptor (EGFR), oncogene (TRK), growth factor receptor-bound protein-7 (GRB7), ras p21 protein activator (RASA2), met proto-oncogene (MET), src-like adapter (SLA), vascular endothelial growth factor (VEGF), vascular endothelial growth factor receptor (VEGFR), nerve growth factor receptor (NGFR), platelet derived growth factor receptor (PDGFR), platelet derived growth factor receptor beta (PDGFRB), dual-specificity tyrosine-(Y)-phosphorylation regulated kinase 2 (DYRK2), dual-specificity tyrosine-(Y)-phosphorylation regulated kinase 3 (DYRK3), dual-specificity tyrosine-(Y)-phosphorylation regulated kinase 4 (DYRK4), dual-specificity tyrosine-(Y)-phosphorylation regulated kinase 1A (DYRK1A), dual-specificity tyrosine-(Y)-phosphorylation regulated kinase 1B (DYRK1B), CDC-like kinase 1 (CLK1), protein tyrosine kinase STY, CDC-like kinase 4 (CLK4), CDC-like kinase 2 (CLK2) or CDC-like kinase 3 (CLK3).

In another embodiment, provided herein are methods for the treatment or prevention of a disease or disorder associated with the modulation, for example inhibition, of serine/threonine kinases or related molecules, including, but not limited to, cyclin-dependent kinase 7 (CDK7), rac serine/threonine protein kinase, serine-threonine protein kinase n (PKN), serine/threonine protein kinase 2 (STK2), zipper protein kinase (ZPK), protein-tyrosine kinase (STY), bruton agammaglobulinemia tyrosine kinase (BTK), mkn28 kinase, protein kinase, x-linked (PRKX), elk-related tyrosine kinase (ERK), ribosomal protein s6 kinase, 90 kd, polypeptide 3 (RPS6KA3), glycogen storage disease VIII, death-associated protein kinase 1 (DAPK1), pctaire protein kinase 1 (PCTK1), protein kinase, interferon-inducible double-stranded ma (PRKR), activin a receptor, type II-like kinase 1 (ACVRLK1), protein kinase, camp-dependent, catalytic, alpha (PRKACA), protein kinase, y-linked (PRKY), G protein-coupled receptor kinase 2 (GPRK21), protein kinase c, theta form (PRKCQ), lim domain kinase 1 (LIMK1), phosphoglycerate kinase 1 PGK1), lim domain kinase 2 (LIMK2), c-jun kinase, activin a receptor, type II-like kinase 2 (ACVRLK2), janus kinase 1 (JAK1), elk1 motif kinase (EMK1), male germ cell-associated kinase (MAK), casein kinase 2, alpha-prime subunit (CSNK2A2), casein kinase 2, beta polypeptide (CSNK2B), casein kinase 2, alpha 1 polypeptide (CSNK2A1), ret proto-oncogene (RET), hematopoietic progenitor kinase 1, conserved helix-loop-helix ubiquitous kinase (CHUK), casein kinase 1, delta (CSNK1D), casein kinase 1, epsilon (CSNK1E), v-akt murine thymoma viral oncogene homolog 1 (AKT1), tumor protein p53 (TP53), protein phosphatase 1, regulatory (inhibitor) subunit 2 (PPP1R2), oncogene pim-1 (PIM1), transforming growth factor-beta receptor, type II (TGFBR2), transforming growth factor-beta receptor, type 1 (TGFBR1), v-raf murine sarcoma viral oncogene homolog b1 (BRAF), bone morphogenetic receptor type II (BMPR2), v-raf murine sarcoma 3611 viral oncogene homolog I (ARAF1), v-raf murine sarcoma 3611 viral oncogene homolog 2 (ARAF2), protein kinase C (PKC), v-kit hardy-zuckerman 4 feline sarcoma viral oncogene homolog (KIT) or c-KIT receptor (KITR).

In another embodiment, provided herein are methods for the treatment or prevention of a disease or disorder associated with the modulation, for example inhibition, of a MAP kinase, including, but not limited to, mitogen-activated protein kinase 3 (MAPK3), p44erk1, p44mapk, mitogen-activated protein kinase 3 (MAP kinase 3; p44), ERK1, PRKM3, P44ERK1, P44MAPK, mitogen-activated protein kinase 1 (MAPK1), mitogen-activated protein kinase kinase 1 (MEK1), MAP2K1protein tyrosine kinase ERK2, mitogen-activated protein kinase 2, extracellular signal-regulated kinase 2, protein tyrosine kinase ERK2, mitogen-activated protein kinase 2, extracellular signal-regulated kinase 2, ERK, p38, p40, p41, ERK2, ERT1, MAPK2, PRKM1, PRKM2, P42MAPK, p41mapk, mitogen-activated protein kinase 7 (MAPK7), BMK1 kinase, extracellular-signal-regulated kinase 5, BMK1, ERK4, ERK5, PRKM7, nemo-like kinase (NLK), likely ortholog of mouse nemo like kinase, mitogen-activated protein kinase 8 (MAPK8), protein kinase JNK1, JNK1 beta protein kinase, JNK1 alpha protein kinase, c-Jun N-terminal kinase 1, stress-activated protein kinase JNK1, JNK, JNK1, PRKM8, SAPK1, JNK1A2, JNK21B1/2, mitogen-activated protein kinase 10 (MAPK10), c-Jun kinase 3, JNK3 alpha protein kinase, c-Jun N-terminal kinase 3, stress activated protein kinase JNK3, stress activated protein kinase beta, mitogen-activated protein kinase 9 (MAPK9), MAP kinase 9, c-Jun kinase 2, c-Jun N-terminal kinase 2, stress-activated protein kinase JNK2, JNK2, JNK2A, JNK2B, PRKM9, JNK-55, JNK2BETA, p54aSAPK, JNK2ALPHA, mitogen-activated protein kinase 14 (MAPK14), p38 MAP kinase, MAP kinase Mxi2, Csaids binding protein, MAX-interacting protein 2, stress-activated protein kinase 2A, p38 mitogen activated protein kinase, cytokine suppressive anti-inflammatory drug binding protein, RK, p38, EXIP, Mxi2, CSBP1, CSBP2, CSPB1, PRKM14, PRKM15, SAPK2A, p38ALPHA, mitogen-activated protein kinase 11 (MAPK11), stress-activated protein kinase-2, stress-activated protein kinase-2b, mitogen-activated protein kinase p38-2, mitogen-activated protein kinase p38beta, P38B, SAPK2, p38-2, PRKM11, SAPK2B, p38Beta, P38BETA2, mitogen-activated protein kinase 13 (MAPK13), stress-activated protein kinase 4, mitogen-activated protein kinase p38 delta, SAPK4, PRKM13, p38delta, mitogen-activated protein kinase 12 (MAPK12), p38gamma, stress-activated protein kinase 3, mitogen-activated protein kinase 3, ERK3, ERK6, SAPK3, PRKM12, SAPK-3, P38GAMMA, mitogen-activated protein kinase 6 (MAPK6), MAP kinase isoform p97, mitogen-activated 5 protein kinase, mitogen-activated 6 protein kinase, extracellular signal-regulated kinase 3, extracellular signal-regulated kinase, p97, ERK3, PRKM6, p97MAPK, mitogen-activated protein kinase 4 (MAPK4), Erk3-related protein kinase, mitogen-activated 4 protein kinase (MAP kinase 4; p63), PRKM4, p63MAPK, ERK3-RELATED or Extracellular signal-regulated kinase 8 (ERK7).

More particularly, cancers and related disorders that can be treated or prevented by methods and compositions provided herein include but are not limited to the following: Leukemias such as but not limited to, acute leukemia, acute lymphocytic leukemia, acute myelocytic leukemias such as myeloblastic, promyelocytic, myelomonocytic, monocytic, erythroleukemia leukemias and myelodysplastic syndrome (or a symptom thereof such as anemia, thrombocytopenia, neutropenia, bicytopenia or pancytopenia), refractory anemia (RA), RA with ringed sideroblasts (RARS), RA with excess blasts (RAEB), RAEB in transformation (RAEB-T), preleukemia and chronic myelomonocytic leukemia (CMML), chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, metastatic cancers, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer, including, but not limited to, adenocarcinoma, lobular (small cell) carcinoma, intraductal carcinoma, medullary breast cancer, mucinous breast cancer, tubular breast cancer, papillary breast cancer, primary cancers, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungaling (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma, gallbladder cancers such as adenocarcinoma; cholangiocarcinomas such as but not limited to pappillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell cancer, adenocarcinoma, hypernephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma,

hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

Accordingly, the methods and compositions provided herein are also useful in the treatment or prevention of a variety of cancers or other abnormal proliferative diseases, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Berketts lymphoma; hematopoietic tumors of myeloid lineage, including acute and chronic myelogenous leukemias and promyelocytic leukemia; tumors of mesenchymal orignin, including fibrosarcoma and rhabdomyoscarcoma; other tumors, including melanoma, seminoma, tetratocarcinoma, neuroblastoma and glioma; tumors of the central and peripheral nervous system, including astrocytoma, glioblastoma multiforme, neuroblastoma, glioma, and schwannomas; solid and blood born tumors; tumors of mesenchymal origin, including fibrosafcoma, rhabdomyoscarama, and osteosarcoma; and other tumors, including melanoma, xenoderma pegmentosum, keratoactanthoma, seminoma, thyroid follicular cancer and teratocarcinoma. It is also contemplated that cancers caused by aberrations in apoptosis would also be treated by the methods and compositions disclosed herein. Such cancers may include but not be limited to follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are treated or prevented in the ovary, bladder, breast, colon, lung, skin, pancreas, or uterus. In other specific embodiments, sarcoma, melanoma, or leukemia is treated or prevented.

In another embodiment, the methods and compositions provided herein are also useful for administration to patients in need of a bone marrow transplant to treat a malignant disease (e.g., patients suffering from acute lymphocytic leukemia, acute myelogenous leukemia, chronic myelogenous leukemia, chronic lymphocytic leukemia, myelodysplastic syndrome (“preleukemia”), monosomy 7 syndrome, non-Hodgkin's lymphoma, neuroblastoma, brain tumors, multiple myeloma, testicular germ cell tumors, breast cancer, lung cancer, ovarian cancer, melanoma, glioma, sarcoma or other solid tumors), those in need of a bone marrow transplant to treat a non-malignant disease (e.g., patients suffering from hematologic disorders, congenital immunodeficiences, mucopolysaccharidoses, lipidoses, osteoporosis, Langerhan's cell histiocytosis, Lesch-Nyhan syndrome or glycogen storage diseases), those undergoing chemotherapy or radiation therapy, those preparing to undergo chemotherapy or radiation therapy and those who have previously undergone chemotherapy or radiation therapy.

In another embodiment, provided herein are methods for the treatment of myeloproliferative disorders or myelodysplastic syndromes, comprising administering to a patient in need thereof an effective amount of an isotopologue of Compound 1 or a composition thereof. In certain embodiments, the myeloproliferative disorder is polycythemia rubra vera; primary thrombocythemia; chronic myelogenous leukemia; acute or chronic granulocytic leukemia; acute or chronic myelomonocytic leukemia; myelofibro-erythroleukemia; or agnogenic myeloid metaplasia.

In another embodiment, provided herein are methods for the treatment of cancer or tumors resistant to other kinase inhibitors such as imatinib mesylate (STI-571 or Gleevec™) treatment, comprising administering to a patient in need thereof an effective amount of an isotopologue of Compound 1 or a composition thereof. In a particular embodiment, provided herein are methods for the treatment of leukemias, including, but not limited to, gastrointestinal stromal tumor (GIST), acute lymphocytic leukemia or chronic myelocytic leukemia resistant to imatinib mesylate (STI-571 or Gleevec™) treatment, comprising administering to a patient in need thereof an effective amount of an isotopologue of Compound 1 or a composition thereof.

In a particular embodiment, provided herein are methods for the treatment or prevention of airway hyperresponsiveness (AHR) and lung inflammation comprising administering an effective amount of an isotopologue of Compound 1 to a patient in need thereof.

In another embodiment, provided herein are methods for the treatment or prevention of a disease or disorder caused, induced or exacerbated by an agonist including, but not limited to, ozone, cold or exercise, comprising administering an effective amount of an isotopologue of Compound 1 to a patient in need thereof. In a specific embodiment, provided herein are methods for the treatment or prevention of asthma, bronchitis, rhinitis, COPD, lung inflammation and AHR caused, induced or exacerbated by an agonist including, but not limited to, ozone, cold or exercise, comprising administering an effective amount of an isotopologue of Compound 1 to a patient in need thereof.

In another embodiment, provided herein are methods for the treatment or prevention of ozone-induced effects (e.g., adverse effects) on a lung, comprising administering an effective amount of an isotopologue of Compound 1 to a patient in need thereof.

In another embodiment, provided herein are methods for modulating inflammatory cell recruitment and/or inflammatory gene expression in the lungs comprising administering an effective amount of an isotopologue of Compound 1 to a patient in need thereof.

In another embodiment, provided herein are methods for inhibiting neutrophil accumulation in bronchoalveolar lavage fluid comprising administering an effective amount of an isotopologue of Compound 1 to a patient in need thereof.

In another embodiment, provided herein are methods for modulating (i.e., inducing or inhibiting) expression of genes involved in oxidative stress response comprising administering an effective amount of an isotopologue of Compound 1 to a patient in need thereof.

In another embodiment, provided herein are methods for modulating (i.e., inducing or inhibiting) expression of genes modulated by ozone comprising administering an effective amount of an isotopologue of Compound 1 to a patient in need thereof. In a specific embodiment, provided herein are methods for modulating (i.e., inducing or inhibiting) expression of the following genes comprising administering an effective amount of an isotopologue of Compound 1 to a patient in need thereof: Interleukin 6; Chemokine (C-X-C motif) ligand 1; A disintegrin-like and metalloprotease (reprolysin type) with thrombospondin type 1 motif, 4; Metallothionein 1; Chemokine (C-X-C motif) ligand 2; Interleukin I receptor, type II; Small chemokine (C-C motif) ligand 11; Pentaxin related gene; Hemopexin; Matrix metalloproteinase 8; Tumor necrosis factor alpha induced protein 6; Cyclin-dependent kinase inhibitor 1A (P21); FK506 binding protein 5; Protease, serine, 22; DNA-damage-inducible transcript 4; Similar to mPLZF(B)=promyelocytic leukemia zinc finger protein {alternatively spliced}(LOC235320), mRNA; Cyclin-dependent kinase inhibitor 1A (P21); Double C2, beta; Suppressor of cytokine signaling 3; Metallothionein 2; Angiopoietin; Solute carrier family 27 (fatty acid transporter), member 3; wingless-related MMTV integration site 7A; BB224790 RIKEN full-length enriched, adult male aorta and vein Mus musculus mRNA sequence; Calcitonin receptor-like; Glia maturation factor, beta; RIKEN full-length enriched library, clone:D230030K09; Mp78a12.x1 Soares_thymus_{—}2NbMT Mus musculus cDNA clone IMAGE:575326 mRNA sequence; cDNA sequence BC025076; Interleukin 6 signal transducer; Myc target 1; Tripartite motif protein 37; Frizzled homolog 2 (Drosophila); DNA segment, Chr 4, Wayne State University 53, expressed; Stromal antigen 2; HLA-B associated transcript 8; RIKEN cDNA 1110014P06 gene; GATA binding protein 3; AV218922 RIKEN full-length enriched, mRNA sequence; and PDZ domain containing, X chromosome.

In one embodiment, provided herein are methods for treating or preventing a disease or disorder treatable or preventable by modulating a kinase pathway, in one embodiment, the JNK pathway, comprising administering an effective amount of an isotopologue of Compound 1 to a patient in need of the treating or preventing. Particular diseases which are treatable or preventable by modulating, for example, inhibiting, a kinase pathway, in one embodiment, the JNK pathway, include, but are not limited to, rheumatoid arthritis; rheumatoid spondylitis; osteoarthritis; gout; asthma, bronchitis; allergic rhinitis; chronic obstructive pulmonary disease; cystic fibrosis; inflammatory bowel disease; irritable bowel syndrome; mucous colitis; ulcerative colitis; Crohn's disease; Huntington's disease; gastritis; esophagitis; hepatitis; pancreatitis; nephritis; multiple sclerosis; lupus erythematosus; Type II diabetes; obesity; atherosclerosis; restenosis following angioplasty; left ventricular hypertrophy; myocardial infarction; stroke; ischemic damages of heart, lung, gut, kidney, liver, pancreas, spleen and brain; acute or chronic organ transplant rejection; preservation of the organ for transplantation; organ failure or loss of limb (e.g., including, but not limited to, that resulting from ischemia-reperfusion injury, trauma, gross bodily injury, car accident, crush injury or transplant failure); graft versus host disease; endotoxin shock; multiple organ failure; psoriasis; burn from exposure to fire, chemicals or radiation; eczema; dermatitis; skin graft; ischemia; ischemic conditions associated with surgery or traumatic injury (e.g., vehicle accident, gunshot wound or limb crush); epilepsy; Alzheimer's disease; Parkinson's disease; immunological response to bacterial or viral infection; cachexia; angiogenic and proliferative dieseases; solid tumor; and cancers of a variety of tissues such as colon, rectum, prostate, liver, lung, bronchus, pancreas, brain, head, neck, stomach, skin, kidney, cervix, blood, larynx, esophagus, mouth, pharynx, urinary bladder, ovary or uterine.

### 4.4 Second Active Agents

A compound provided herein, or a pharmaceutically acceptable salt, solvate, prodrug, clathrate, or stereoisomer thereof, can be combined with other pharmacologically active compounds (“second active agents”) in methods and compositions provided herein. Certain combinations may work synergistically in the treatment of particular types diseases or disorders, and conditions and symptoms associated with such diseases or disorders. A compound provided herein, or a pharmaceutically acceptable salt, solvate, clathrate, stereoisomer or prodrug thereof, can also work to alleviate adverse effects associated with certain second active agents, and vice versa.

One or more second active ingredients or agents can be used in the methods and compositions provided herein. Second active agents can be large molecules (e.g., proteins) or small molecules (e.g., synthetic inorganic, organometallic, or organic molecules).

Examples of large molecule active agents include, but are not limited to, hematopoietic growth factors, cytokines, and monoclonal and polyclonal antibodies. Specific examples of the active agents are anti-CD40 monoclonal antibodies (such as, for example, SGN-40); histone deacetylyase inhibitors (such as, for example, SAHA and LAQ 824); heat-shock protein-90 inhibitors (such as, for example, 17-AAG); insulin-like growth factor-1 receptor kinase inhibitors; vascular endothelial growth factor receptor kinase inhibitors (such as, for example, PTK787); insulin growth factor receptor inhibitors; lysophosphatidic acid acyltransrerase inhibitors; IkB kinase inhibitors; p38MAPK inhibitors; EGFR inhibitors (such as, for example, gefitinib and erlotinib HCL); HER-2 antibodies (such as, for example, trastuzumab (Herceptin®) and pertuzumab (Omnitarg™)); VEGFR antibodies (such as, for example, bevacizumab (Avastin™)); VEGFR inhibitors (such as, for example, flk-1 specific kinase inhibitors, SU5416 and ptk787/zk222584); PI3K inhibitors (such as, for example, wortmannin); C-Met inhibitors (such as, for example, PHA-665752); monoclonal antibodies (such as, for example, rituximab (Rituxan®), tositumomab (Bexxar®), edrecolomab (Panorex®) and G250); and anti-TNF-α antibodies. Examples of small molecule active agents include, but are not limited to, anticancer agents and antibiotics (e.g., clarithromycin).

Specific second active compounds that can be combined with compounds provided herein vary depending on the specific indication to be treated, prevented or managed.

For instance, for the treatment, prevention or management of cancer, second active agents include, but are not limited to: semaxanib; cyclosporin; etanercept; doxycycline; bortezomib; acivicin; aclarubicin; acodazole hydrochloride; acronine; adozelesin; aldesleukin; altretamine; ambomycin; ametantrone acetate; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other second agents include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dihydrotaxol; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (Gleevec®), imiquimod; immunostimulant peptides; insulin-like growth factor-1 receptor inhibitor; interferon agonists; interferons; interleukins; iobenguane; iododoxorubicin; 4-ipomeanol; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim; Erbitux, human chorionic gonadotrophin; monophosphoryl lipid A+myobacterium cell wall sk; mopidamol; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; nilutamide; nisamycin; nitric oxide modulators; nitroxide antioxidant; nitrullyn; oblimersen (Genasense®); O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; R11 retinamide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer.

Specific second active agents include, but are not limited to, 2-methoxyestradiol, telomestatin, inducers of apoptosis in mutiple myeloma cells (such as, for example, TRAIL), statins, semaxanib, cyclosporin, etanercept, doxycycline, bortezomib, oblimersen (Genasense®), remicade, docetaxel, celecoxib, melphalan, dexamethasone (Decadron®), steroids, gemcitabine, cisplatinum, temozolomide, etoposide, cyclophosphamide, temodar, carboplatin, procarbazine, gliadel, tamoxifen, topotecan, methotrexate, Arisa®, taxol, taxotere, fluorouracil, leucovorin, irinotecan, xeloda, CPT-11, interferon alpha, pegylated interferon alpha (e.g., PEG INTRON-A), capecitabine, cisplatin, thiotepa, fludarabine, carboplatin, liposomal daunorubicin, cytarabine, doxetaxol, pacilitaxel, vinblastine, IL-2, GM-CSF, dacarbazine, vinorelbine, zoledronic acid, palmitronate, biaxin, busulphan, prednisone, bisphosphonate, arsenic trioxide, vincristine, doxorubicin (Doxil®), paclitaxel, ganciclovir, adriamycin, estramustine sodium phosphate (Emcyt®), sulindac, and etoposide.

In another embodiment, examples of specific second agents according to the indications to be treated, prevented, or managed can be found in the following references, all of which are incorporated herein in their entireties: U.S. Pat. Nos. 6,281,230 and 5,635,517; U.S. publication nos. 2004/0220144, 2004/0190609, 2004/0087546, 2005/0203142, 2004/0091455, 2005/0100529, 2005/0214328, 2005/0239842, 2006/0154880, 2006/0122228, and 2005/0143344; and U.S. provisional application No. 60/631,870.

Examples of second active agents that may be used for the treatment, prevention and/or management of pain include, but are not limited to, conventional therapeutics used to treat or prevent pain such as antidepressants, anticonvulsants, antihypertensives, anxiolytics, calcium channel blockers, muscle relaxants, non-narcotic analgesics, opioid analgesics, anti-inflammatories, cox-2 inhibitors, immunomodulatory agents, alpha-adrenergic receptor agonists or antagonists, immunosuppressive agents, corticosteroids, hyperbaric oxygen, ketamine, other anesthetic agents, NMDA antagonists, and other therapeutics found, for example, in the Physician's Desk Reference 2003. Specific examples include, but are not limited to, salicylic acid acetate (Aspirin®), celecoxib (Celebrex®), Enbrel®, ketamine, gabapentin (Neurontin®), phenyloin (Dilantin®), carbamazepine (Tegretol®), oxcarbazepine (Trileptal®), valproic acid (Depakene®), morphine sulfate, hydromorphone, prednisone, griseofulvin, penthonium, alendronate, dyphenhydramide, guanethidine, ketorolac (Acular®), thyrocalcitonin, dimethylsulfoxide (DMSO), clonidine (Catapress®), bretylium, ketanserin, reserpine, droperidol, atropine, phentolamine, bupivacaine, lidocaine, acetaminophen, nortriptyline (Pamelor®), amitriptyline (Elavil®), imipramine (Tofranil®), doxepin (Sinequan®), clomipramine (Anafranil®), fluoxetine (Prozac®), sertraline (Zoloft®), naproxen, nefazodone (Serzone®), venlafaxine (Effexor®), trazodone (Desyrel®), bupropion (Wellbutrin®), mexiletine, nifedipine, propranolol, tramadol, lamotrigine, vioxx, ziconotide, ketamine, dextromethorphan, benzodiazepines, baclofen, tizanidine and phenoxybenzamine.

Examples of second active agents that may be used for the treatment, prevention and/or management of macular degeneration and related syndromes include, but are not limited to, a steroid, a light sensitizer, an integrin, an antioxidant, an interferon, a xanthine derivative, a growth hormone, a neutrotrophic factor, a regulator of neovascularization, an anti-VEGF antibody, a prostaglandin, an antibiotic, a phytoestrogen, an anti-inflammatory compound or an antiangiogenesis compound, or a combination thereof. Specific examples include, but are not limited to, verteporfin, purlytin, an angiostatic steroid, rhuFab, interferon-2α, pentoxifylline, tin etiopurpurin, motexafin, lucentis, lutetium, 9-fluoro-11,21-dihydroxy-16,17-1-methylethylidinebis(oxy)pregna-1,4-diene-3,20-dione, latanoprost (see U.S. Pat. No. 6,225,348), tetracycline and its derivatives, rifamycin and its derivatives, macrolides, metronidazole (U.S. Pat. Nos. 6,218,369 and 6,015,803), genistein, genistin, 6′-O-Mal genistin, 6′-O-Ac genistin, daidzein, daidzin, 6′-O-Mal daidzin, 6′-O-Ac daidzin, glycitein, glycitin, 6′-O-Mal glycitin, biochanin A, formononetin (U.S. Pat. No. 6,001,368), triamcinolone acetomide, dexamethasone (U.S. Pat. No. 5,770,589), glutathione (U.S. Pat. No. 5,632,984), basic fibroblast growth factor (bFGF), transforming growth factor b (TGF-b), brain-derived neurotrophic factor (BDNF), plasminogen activator factor type 2 (PAI-2), EYE101 (Eyetech Pharmaceuticals), LY333531 (Eli Lilly), Miravant, and RETISERT implant (Bausch & Lomb). All of the references cited herein are incorporated in their entireties by reference.

Examples of second active agents that may be used for the treatment, prevention and/or management of skin diseases include, but are not limited to, keratolytics, retinoids, α-hydroxy acids, antibiotics, collagen, botulinum toxin, interferon, steroids, and immunomodulatory agents. Specific examples include, but are not limited to, 5-fluorouracil, masoprocol, trichloroacetic acid, salicylic acid, lactic acid, ammonium lactate, urea, tretinoin, isotretinoin, antibiotics, collagen, botulinum toxin, interferon, corticosteroid, transretinoic acid and collagens such as human placental collagen, animal placental collagen, Dermalogen, AlloDerm, Fascia, Cymetra, Autologen, Zyderm, Zyplast, Resoplast, and Isolagen.

Examples of second active agents that may be used for the treatment, prevention and/or management of pulmonary hepertension and related disorders include, but are not limited to, anticoagulants, diuretics, cardiac glycosides, calcium channel blockers, vasodilators, prostacyclin analogues, endothelin antagonists, phosphodiesterase inhibitors (e.g., PDE V inhibitors), endopeptidase inhibitors, lipid lowering agents, thromboxane inhibitors, and other therapeutics known to reduce pulmonary artery pressure. Specific examples include, but are not limited to, warfarin (Coumadin®), a diuretic, a cardiac glycoside, digoxin-oxygen, diltiazem, nifedipine, a vasodilator such as prostacyclin (e.g., prostaglandin 12 (PGI2), epoprostenol (EPO, Floran®), treprostinil (Remodulin®), nitric oxide (NO), bosentan (Tracleer®), amlodipine, epoprostenol (Floran®), treprostinil (Remodulin®), prostacyclin, tadalafil (Clalis®), simvastatin (Zocor®), omapatrilat (Vanlev®), irbesartan (Avapro®), pravastatin (Pravachol®), digoxin, L-arginine, iloprost, betaprost, and sildenafil (Viagra®).

Examples of second active agents that may be used for the treatment, prevention and/or management of asbestos-related disorders include, but are not limited to, anthracycline, platinum, alkylating agent, oblimersen (Genasense®), cisplatinum, cyclophosphamide, temodar, carboplatin, procarbazine, gliadel, tamoxifen, topotecan, methotrexate, taxotere, irinotecan, capecitabine, cisplatin, thiotepa, fludarabine, carboplatin, liposomal daunorubicin, cytarabine, doxetaxol, pacilitaxel, vinblastine, IL-2, GM-CSF, dacarbazine, vinorelbine, zoledronic acid, palmitronate, biaxin, busulphan, prednisone, bisphosphonate, arsenic trioxide, vincristine, doxorubicin (Doxil®), paclitaxel, ganciclovir, adriamycin, bleomycin, hyaluronidase, mitomycin C, mepacrine, thiotepa, tetracycline and gemcitabine.

Examples of second active agents that may be used for the treatment, prevention and/or management of parasitic diseases include, but are not limited to, chloroquine, quinine, quinidine, pyrimethamine, sulfadiazine, doxycycline, clindamycin, mefloquine, halofantrine, primaquine, hydroxychloroquine, proguanil, atovaquone, azithromycin, suramin, pentamidine, melarsoprol, nifurtimox, benznidazole, amphotericin B, pentavalent antimony compounds (e.g., sodium stiboglucuronate), interferon gamma, itraconazole, a combination of dead promastigotes and BCG, leucovorin, corticosteroids, sulfonamide, spiramycin, IgG (serology), trimethoprim, and sulfamethoxazole.

Examples of second active agents that may be used for the treatment, prevention and/or management of immunodeficiency disorders include, but are not limited to: antibiotics (therapeutic or prophylactic) such as, but not limited to, ampicillin, tetracycline, penicillin, cephalosporins, streptomycin, kanamycin, and erythromycin; antivirals such as, but not limited to, amantadine, rimantadine, acyclovir, and ribavirin; immunoglobulin; plasma; immunologic enhancing drugs such as, but not limited to, levami sole and isoprinosine; biologics such as, but not limited to, gammaglobulin, transfer factor, interleukins, and interferons; hormones such as, but not limited to, thymic; and other immunologic agents such as, but not limited to, B cell stimulators (e.g., BAFF/BlyS), cytokines (e.g., IL-2, IL-4, and IL-5), growth factors (e.g., TGF-α), antibodies (e.g., anti-CD40 and IgM), oligonucleotides containing unmethylated CpG motifs, and vaccines (e.g., viral and tumor peptide vaccines).

Examples of second active agents that may be used for the treatment, prevention and/or management of CNS disorders include, but are not limited to: opioids; a dopamine agonist or antagonist, such as, but not limited to, Levodopa, L-DOPA, cocaine, α-methyl-tyrosine, reserpine, tetrabenazine, benzotropine, pargyline, fenodolpam mesylate, cabergoline, pramipexole dihydrochloride, ropinorole, amantadine hydrochloride, selegiline hydrochloride, carbidopa, pergolide mesylate, Sinemet CR, and Symmetrel; a MAO inhibitor, such as, but not limited to, iproniazid, clorgyline, phenelzine and isocarboxazid; a COMT inhibitor, such as, but not limited to, tolcapone and entacapone; a cholinesterase inhibitor, such as, but not limited to, physostigmine saliclate, physostigmine sulfate, physostigmine bromide, meostigmine bromide, neostigmine methylsulfate, ambenonim chloride, edrophonium chloride, tacrine, pralidoxime chloride, obidoxime chloride, trimedoxime bromide, diacetyl monoxim, endrophonium, pyridostigmine, and demecarium; an anti-inflammatory agent, such as, but not limited to, naproxen sodium, diclofenac sodium, diclofenac potassium, celecoxib, sulindac, oxaprozin, diflunisal, etodolac, meloxicam, ibuprofen, ketoprofen, nabumetone, refecoxib, methotrexate, leflunomide, sulfasalazine, gold salts, Rho-D Immune Globulin, mycophenylate mofetil, cyclosporine, azathioprine, tacrolimus, basiliximab, daclizumab, salicylic acid, acetylsalicylic acid, methyl salicylate, diflunisal, salsalate, olsalazine, sulfasalazine, acetaminophen, indomethacin, sulindac, mefenamic acid, meclofenamate sodium, tolmetin, ketorolac, dichlofenac, flurbinprofen, oxaprozin, piroxicam, meloxicam, ampiroxicam, droxicam, pivoxicam, tenoxicam, phenylbutazone, oxyphenbutazone, antipyrine, aminopyrine, apazone, zileuton, aurothioglucose, gold sodium thiomalate, auranofin, methotrexate, colchicine, allopurinol, probenecid, sulfinpyrazone and benzbromarone or betamethasone and other glucocorticoids; and an antiemetic agent, such as, but not limited to, metoclopromide, domperidone, prochlorperazine, promethazine, chlorpromazine, trimethobenzamide, ondansetron, granisetron, hydroxyzine, acetylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinol, thiethylperazine, thioproperazine, tropisetron, and a mixture thereof.

Examples of second active agents that may be used for the treatment, prevention and/or management of CNS injuries and related syndromes include, but are not limited to, immunomodulatory agents, immunosuppressive agents, antihypertensives, anticonvulsants, fibrinolytic agents, antiplatelet agents, antipsychotics, antidepressants, benzodiazepines, buspirone, amantadine, and other known or conventional agents used in patients with CNS injury/damage and related syndromes. Specific examples include, but are not limited to: steroids (e.g., glucocorticoids, such as, but not limited to, methylprednisolone, dexamethasone and betamethasone); an anti-inflammatory agent, including, but not limited to, naproxen sodium, diclofenac sodium, diclofenac potassium, celecoxib, sulindac, oxaprozin, diflunisal, etodolac, meloxicam, ibuprofen, ketoprofen, nabumetone, refecoxib, methotrexate, leflunomide, sulfasalazine, gold salts, RHo-D Immune Globulin, mycophenylate mofetil, cyclosporine, azathioprine, tacrolimus, basiliximab, daclizumab, salicylic acid, acetylsalicylic acid, methyl salicylate, diflunisal, salsalate, olsalazine, sulfasalazine, acetaminophen, indomethacin, sulindac, mefenamic acid, meclofenamate sodium, tolmetin, ketorolac, dichlofenac, flurbinprofen, oxaprozin, piroxicam, meloxicam, ampiroxicam, droxicam, pivoxicam, tenoxicam, phenylbutazone, oxyphenbutazone, antipyrine, aminopyrine, apazone, zileuton, aurothioglucose, gold sodium thiomalate, auranofin, methotrexate, colchicine, allopurinol, probenecid, sulfinpyrazone and benzbromarone; a cAMP analog including, but not limited to, db-cAMP; an agent comprising a methylphenidate drug, which comprises 1-threo-methylphenidate, d-threo-methylphenidate, dl-threo-methylphenidate, 1-erythro-methylphenidate, d-erythro-methylphenidate, dl-erythro-methylphenidate, and a mixture thereof; and a diuretic agent such as, but not limited to, mannitol, furosemide, glycerol, and urea.

Examples of second active agent that may be used for the treatment, prevention and/or management of dysfunctional sleep and related syndromes include, but are not limited to, a tricyclic antidepressant agent, a selective serotonin reuptake inhibitor, an antiepileptic agent (gabapentin, pregabalin, carbamazepine, oxcarbazepine, levitiracetam, topiramate), an antiaryhthmic agent, a sodium channel blocking agent, a selective inflammatory mediator inhibitor, an opioid agent, a second immunomodulatory compound, a combination agent, and other known or conventional agents used in sleep therapy. Specific examples include, but are not limited to, Neurontin, oxycontin, morphine, topiramate, amitryptiline, nortryptiline, carbamazepine, Levodopa, L-DOPA, cocaine, α-methyl-tyrosine, reserpine, tetrabenazine, benzotropine, pargyline, fenodolpam mesylate, cabergoline, pramipexole dihydrochloride, ropinorole, amantadine hydrochloride, selegiline hydrochloride, carbidopa, pergolide mesylate, Sinemet CR, Symmetrel, iproniazid, clorgyline, phenelzine, isocarboxazid, tolcapone, entacapone, physostigmine saliclate, physostigmine sulfate, physostigmine bromide, meostigmine bromide, neostigmine methylsulfate, ambenonim chloride, edrophonium chloride, tacrine, pralidoxime chloride, obidoxime chloride, trimedoxime bromide, diacetyl monoxim, endrophonium, pyridostigmine, demecarium, naproxen sodium, diclofenac sodium, diclofenac potassium, celecoxib, sulindac, oxaprozin, diflunisal, etodolac, meloxicam, ibuprofen, ketoprofen, nabumetone, refecoxib, methotrexate, leflunomide, sulfasalazine, gold salts, RHo-D Immune Globulin, mycophenylate mofetil, cyclosporine, azathioprine, tacrolimus, basiliximab, daclizumab, salicylic acid, acetylsalicylic acid, methyl salicylate, diflunisal, salsalate, olsalazine, sulfasalazine, acetaminophen, indomethacin, sulindac, mefenamic acid, meclofenamate sodium, tolmetin, ketorolac, dichlofenac, flurbinprofen, oxaprozin, piroxicam, meloxicam, ampiroxicam, droxicam, pivoxicam, tenoxicam, phenylbutazone, oxyphenbutazone, antipyrine, aminopyrine, apazone, zileuton, aurothioglucose, gold sodium thiomalate, auranofin, methotrexate, colchicine, allopurinol, probenecid, sulfinpyrazone, benzbromarone, betamethasone and other glucocorticoids, metoclopromide, domperidone, prochlorperazine, promethazine, chlorpromazine, trimethobenzamide, ondansetron, granisetron, hydroxyzine, acetylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinol, thiethylperazine, thioproperazine, tropisetron, and a mixture thereof.

Examples of second active agents that may be used for the treatment, prevention and/or management of hemoglobinopathy and related disorders include, but are not limited to: interleukins, such as IL-2 (including recombinant IL-II (“rIL2”) and canarypox IL-2), IL-10, IL-12, and IL-18; interferons, such as interferon alfa-2a, interferon alfa-2b, interferon alfa-n1, interferon alfa-n3, interferon beta-I a, and interferon gamma-I b; and G-CSF; hydroxyurea; butyrates or butyrate derivatives; nitrous oxide; hydroxy urea; HEMOXIN™ (NIPRISAN™; see U.S. Pat. No. 5,800,819); Gardos channel antagonists such as clotrimazole and triaryl methane derivatives; Deferoxamine; protein C; and transfusions of blood, or of a blood substitute such as Hemospan™ or Hemospan™ PS (Sangart).

Administration of a compound provided herein, or a pharmaceutically acceptable salt, solvate, clathrate, stereoisomer or prodrug thereof, and the second active agents to a patient can occur simultaneously or sequentially by the same or different routes of administration. The suitability of a particular route of administration employed for a particular active agent will depend on the active agent itself (e.g., whether it can be administered orally without decomposing prior to entering the blood stream) and the disease being treated. One of administration for compounds provided herein is oral. Routes of administration for the second active agents or ingredients are known to those of ordinary skill in the art. See, e.g., Physicians' Desk Reference (60^{th} ed., 2006).

In one embodiment, the second active agent is administered intravenously or subcutaneously and once or twice daily in an amount of from about 1 to about 1000 mg, from about 5 to about 500 mg, from about 10 to about 350 mg, or from about 50 to about 200 mg. The specific amount of the second active agent will depend on the specific agent used, the type of disease being treated or managed, the severity and stage of disease, and the amount(s) of compounds provided herein and any optional additional active agents concurrently administered to the patient.

As discussed elsewhere herein, also encompassed is a method of reducing, treating and/or preventing adverse or undesired effects associated with conventional therapy including, but not limited to, surgery, chemotherapy, radiation therapy, hormonal therapy, biological therapy and immunotherapy. Compounds provided herein and other active ingredients can be administered to a patient prior to, during, or after the occurrence of the adverse effect associated with conventional therapy.

### 4.5 Pharmaceutical Compositions and Dosage Forms

The isotopologues of Compound 1 can be administered to a patient orally or parenterally in the conventional form of preparations, such as capsules, microcapsules, tablets, granules, powder, troches, pills, suppositories, injections, suspensions and syrups. Suitable formulations can be prepared by methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g, sodium benzoate, sodium bisulfite, methylparaben or propylparaben), a stabilizer (e.g., citric acid, sodium citrate or acetic acid), a suspending agent (e.g., methylcellulose, polyvinyl pyrroliclone or aluminum stearate), a dispersing agent (e.g., hydroxypropylmethylcellulose), a diluent (e.g., water), and base wax (e.g., cocoa butter, white petrolatum or polyethylene glycol). The effective amount of the isotopologues of Compound 1 in the pharmaceutical composition may be at a level that will exercise the desired effect; for example, about 0.005 mg/kg of a patient's body weight to about 10 mg/kg of a patient's body weight in unit dosage for both oral and parenteral administration.

The dose of an isotopologue of Compound 1 to be administered to a patient is rather widely variable and can be subject to the judgment of a health-care practitioner. In general, the isotopologues of Compound 1 can be administered one to four times a day in a dose of about 0.005 mg/kg of a patient's body weight to about 10 mg/kg of a patient's body weight in a patient, but the above dosage may be properly varied depending on the age, body weight and medical condition of the patient and the type of administration. In one embodiment, the dose is about 0.01 mg/kg of a patient's body weight to about 5 mg/kg of a patient's body weight, about 0.05 mg/kg of a patient's body weight to about 1 mg/kg of a patient's body weight, about 0.1 mg/kg of a patient's body weight to about 0.75 mg/kg of a patient's body weight or about 0.25 mg/kg of a patient's body weight to about 0.5 mg/kg of a patient's body weight. In one embodiment, one dose is given per day. In any given case, the amount of the isotopologue of Compound 1 administered will depend on such factors as the solubility of the active component, the formulation used and the route of administration.

In another embodiment, provided herein are methods for the treatment or prevention of a disease or disorder comprising the administration of about 0.375 mg/day to about 750 mg/day, about 0.75 mg/day to about 375 mg/day, about 3.75 mg/day to about 75 mg/day, about 7.5 mg/day to about 55 mg/day or about 18 mg/day to about 37 mg/day of an isotopologue of Compound 1 to a patient in need thereof.

In another embodiment, provided herein are methods for the treatment or prevention of a disease or disorder comprising the administration of about 1 mg/day to about 1200 mg/day, about 10 mg/day to about 1200 mg/day, about 100 mg/day to about 1200 mg/day, about 400 mg/day to about 1200 mg/day, about 600 mg/day to about 1200 mg/day, about 400 mg/day to about 800 mg/day or about 600 mg/day to about 800 mg/day of an isotopologue of Compound 1 to a patient in need thereof. In a particular embodiment, the methods disclosed herein comprise the administration of 400 mg/day, 600 mg/day or 800 mg/day of an isotopologue of Compound 1 to a patient in need thereof.

In another embodiment, provided herein are unit dosage formulations that comprise between about 1 mg and 200 mg, about 35 mg and about 1400 mg, about 125 mg and about 1000 mg, about 250 mg and about 1000 mg, or about 500 mg and about 1000 mg of an isotopologue of Compound 1.

In a particular embodiment, provided herein are unit dosage formulation comprising about 100 mg or 400 mg of an isotopologue of Compound 1.

In another embodiment, provided herein are unit dosage formulations that comprise 1 mg, 5 mg, 10 mg, 15 mg, 20 mg, 30 mg, 35 mg, 50 mg, 70 mg, 100 mg, 125 mg, 140 mg, 175 mg, 200 mg, 250 mg, 280 mg, 350 mg, 500 mg, 560 mg, 700 mg, 750 mg, 1000 mg or 1400 mg of an isotopologue of Compound 1.

An isotopologue of Compound 1 can be administered once, twice, three, four or more times daily. In a particular embodiment, doses of 600 mg or less are administered as a once daily dose and doses of more than 600 mg are administered twice daily in an amount equal to one half of the total daily dose.

An isotopologue of Compound 1 can be administered orally for reasons of convenience. In one embodiment, when administered orally, an isotopologue of Compound 1 is administered with a meal and water. In another embodiment, the isotopologue of Compound 1 is dispersed in water or juice (e.g., apple juice or orange juice) and administered orally as a suspension.

The isotopologue of Compound 1 can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. The mode of administration is left to the discretion of the health-care practitioner, and can depend in-part upon the site of the medical condition.

In one embodiment, provided herein are capsules containing an isotopologue of Compound 1 without an additional carrier, excipient or vehicle.

In another embodiment, provided herein are compositions comprising an effective amount of an isotopologue of Compound 1 and a pharmaceutically acceptable carrier or vehicle, wherein a pharmaceutically acceptable carrier or vehicle can comprise an excipient, diluent, or a mixture thereof. In one embodiment, the composition is a pharmaceutical composition.

The compositions can be in the form of tablets, chewable tablets, capsules, solutions, parenteral solutions, troches, suppositories and suspensions and the like. Compositions can be formulated to contain a daily dose, or a convenient fraction of a daily dose, in a dosage unit, which may be a single tablet or capsule or convenient volume of a liquid. In one embodiment, the solutions are prepared from water-soluble salts, such as the hydrochloride salt. In general, all of the compositions are prepared according to known methods in pharmaceutical chemistry. Capsules can be prepared by mixing an isotopologue of Compound 1 with a suitable carrier or diluent and filling the proper amount of the mixture in capsules. The usual carriers and diluents include, but are not limited to, inert powdered substances such as starch of many different kinds, powdered cellulose, especially crystalline and microcrystalline cellulose, sugars such as fructose, mannitol and sucrose, grain flours and similar edible powders.

Tablets can be prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant might be necessary in a tablet formulation to prevent the tablet and punches from sticking in the die. The lubricant can be chosen from such slippery solids as talc, magnesium and calcium stearate, stearic acid and hydrogenated vegetable oils. Tablet disintegrators are substances that swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethyl cellulose, for example, can be used as well as sodium lauryl sulfate. Tablets can be coated with sugar as a flavor and sealant, or with film-forming protecting agents to modify the dissolution properties of the tablet. The compositions can also be formulated as chewable tablets, for example, by using substances such as mannitol in the formulation.

When it is desired to administer an isotopologue of Compound 1 as a suppository, typical bases can be used. Cocoa butter is a traditional suppository base, which can be modified by addition of waxes to raise its melting point slightly. Water-miscible suppository bases comprising, particularly, polyethylene glycols of various molecular weights are in wide use.

The effect of the isotopologue of Compound 1 can be delayed or prolonged by proper formulation. For example, a slowly soluble pellet of the isotopologue of Compound 1 can be prepared and incorporated in a tablet or capsule, or as a slow-release implantable device. The technique also includes making pellets of several different dissolution rates and filling capsules with a mixture of the pellets. Tablets or capsules can be coated with a film that resists dissolution for a predictable period of time. Even the parenteral preparations can be made long-acting, by dissolving or suspending the isotopologue of Compound 1 in oily or emulsified vehicles that allow it to disperse slowly in the serum.

### 5. EXAMPLES

General:

Isotopically enriched analogs of the compounds provided herein may generally be prepared according known procedures for the synthesis of Compound 1, wherein one or more of the reagents, starting materials, precursors, or intermediates used is replaced by one or more isotopically enriched reagents, starting materials, precursors, or intermediates. Isotopically enriched reagents, starting materials, precursors, or intermediates are commercially available or may be prepared by routine procedures known to one of skill in the art. Schemes for the preparation of exemplary isotopically enriched compounds are illustrated below.

### Abbreviations

AcOH: acetic acid

AP: area purity

CP: chemical purity

DCM: dichloromethane

DIEA: diisopropylamine

DMSO: dimethylsulfoxide

EDC: 1-ethyl-3-(3-dimethylaminopropyl)carbodiimide

EtOAc: ethyl acetate

EtOH: ethanol

HPLC: high performance liquid chromatography

i-PrOH: isopropanol

LC-MS: liquid chromatography/mass spectrometry

MeCN: acetonitrile

MeOH: methanol

MTBE: methyl tert-butyl ether

NMR: nuclear magnetic resonance

PMA: phosphomolybdic acid

RCP: radiochemical purity

RT: retention time

TEA: triethylamine

TFA: trifluoroacetic acid

THF: tetrahydrofuran

TLC: thin layer chromatography

Trt: trityl

### 5.1 Example 1

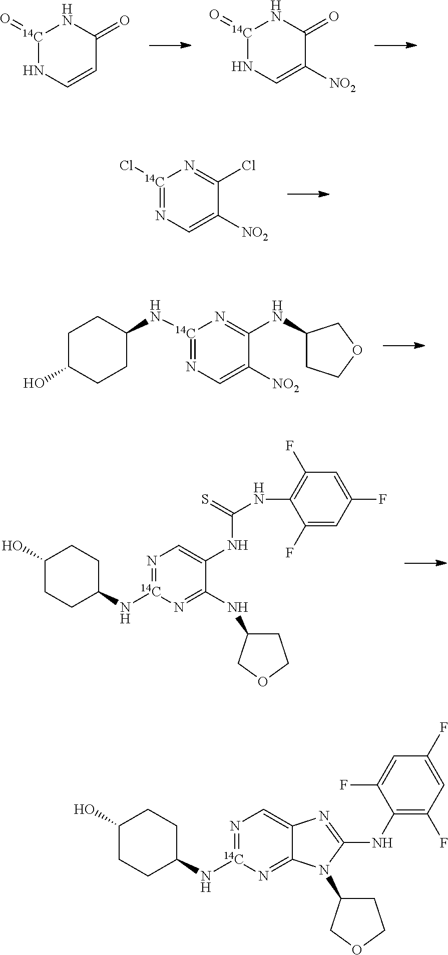 [SMILES: O=c1cc[nH][14c](=O)[nH]1 ; O=c1[nH][14c](=O)[nH]cc1[N+](=O)[O-] ; O=[N+]([O-])c1cn[14c](Cl)nc1Cl ; O=[N+]([O-])c1cn[14c](N[C@H]2CC[C@H](O)CC2)nc1N[C@@H]1CCOC1 ; O[C@H]1CC[C@H](N[14c]2ncc(NC(=S)Nc3c(F)cc(F)cc3F)c(N[C@H]3CCOC3)n2)CC1 ; O[C@H]1CC[C@H](N[14c]2ncc3nc(Nc4c(F)cc(F)cc4F)n([C@H]4CCOC4)c3n2)CC1]

All reagents and solvents were used as received from commercial sources unless otherwise stated. Cold synthesis of the chemical steps was performed on scale prior to the hot synthesis. The various reactions were run under nitrogen atmosphere, unless stated otherwise and anhydrous solvents were transferred under nitrogen gas via syringe. Uracil[2-14C], 198 mCi, specific activity 56.0 mCi/mmol was utilized to incorporate the carbon-14 label. Flash chromatography was performed using silica gel 60 (230-400 mesh). Thin layer chromatography (TLC) was performed on pre-coated (250 glass-backed Silica Gel GF Uniplates (2.5×10 cm) and 250 m Silica Gel 60 F₂₅₄ plates (5×10 cm) from EM Science. TLC spots were visualized under ultraviolet light (254 nm) and/or heating with phosphomolybdic acid stain (PMA): 3-5% PMA in methanol. Radio TLC plates were scanned on a BIOSCAN System AR2000 Imaging Scanner using P-10 gas (90% argon, 10% methane). The identities of the products were confirmed by co-migration with standards. via TLC and/or HPLC retention times. Radio-HPLC analyses of intermediates (condition A) were conducted on a Waters 600 Multisolvent Delivery System equipped with a Waters 996 Photodiode Array, Beta-Ram Radioactive Flow-Through Monitor System, Model 2 (IN/US Systems Inc.) and Millennium 32 software. The preparative HPLC purification of the target compound (condition B) was performed on a Waters Delta Prep 4000 Preparative Chromatography System equipped with a Waters 4000 System Controller and a Waters Tunable Absorbance Detector. HPLC radiochemical and chemical purity of the target compound for release were performed (condition C) using a Waters 2695 Separations Module, Waters 2487 Absorbance Detector, Beta-Ram Radioactive Flow-Through Monitor System, Model 2 (IN/US Systems, Inc.) and Empower software. Radioactivity quantification was obtained with a Packard Minaxi Tri-Carb Liquid Scintillation Analyzer (Model 1600 TR) using Bio-Safe II scintillation cocktail.

HPLC Conditions A:

Luna C₁₈ (2) column, 3μ, 4.6×150 mm, Mobile phases: A water (0.05% TFA), B acetonitrile. Timetable (% A/% B): 0 min 90/10, 12 min 10/90, 13 min 5/95, 20 min 5/95. Flow rate 1.0 mL/min. Scintillant flow rate 1.0 mL/min. UV detection at 254 nm. Column temperature: room temperature.

HPLC Conditions B:

Luna C₁₈ (2) column, 3μ, 21.2×250 mm. Mobile phases: A 0.8 mM NH₄OH/water, B acetonitrile. Timetable (% A/% B) 0 min 80/20, 6 min 80/20, 22 min 5/95, 25 min 5/95, 26 min 80/20. Flow rate 12.0 mL/min. UV detection at 243 nm. Column temperature: room temperature.

HPLC Conditions C:

Luna C₁₈ (2) column, 5μ, 4.6×150 mm. Mobile phases: A water/acetonitrile (95:5) (0.1% TFA), B water/acetonitrile (10/90) (0.1% TFA). Timetable (% A/% B) 0 min 97/3, 2 min 97/3, 8 min 85/15, 30 min 78/22, 50 min 35/65, 51 min 97/3, 55 min 97/3. Flow rate 1.0 mL/min. Scintillant flow rate 1.0 mL/min. UV detection at 243 nm. Column temperature: 40° C.

5-Nitrouracil [¹⁴C]:

A mixture of uracil[2-¹⁴C](198 mCi, specific activity 56 mCi/mmol, 403 mg, 3.5 mmol), fuming nitric acid (1.5 mL) and concentrated sulfuric acid (0.2 mL) was warmed (55° C. bath) with stirring in a round bottom flask with a drierite tube. The temperature was maintained for approximately 4 h, then the reaction was cooled to room temperature. The reaction mixture was poured over ice (5 g), the solids were filtered and washed with water (15 mL) and dried under reduced pressure to provide the target compound as a white solid (441 mg, 78%). TLC [MeOH/DCM (1:3)] Rf=0.41. The compound was used in the next step without further purification.

2,4-Dichloro-5-nitropyrimidine[¹⁴C].

A mixture of the compound obtained above (441 mg, 2.8 mmol), POCl₃ (2.5 mL, 26.8 mmol) and dimethylaniline (0.6 mL, 4.7 mmol) was warmed (110° C.) under an atmosphere of nitrogen. After approximately 4 h, excess POCl₃ was removed under reduced pressure and the crude reaction mixture dissolved in DCM (5 mL). The mixture was pulled through a plug of silica and the plug was washed with DCM (35 mL). The filtrates were combined and evaporated to dryness under vacuum to afford the target compound as a dark viscous oil. TLC [EtOAc/hexanes (1:9)] Rf=0.39. The compound was used in the next step without further purification.

4-[5-Nitro-4-((R)-tetrahydrofuran-3-ylamino)-pyrimidin-2-ylamino]-cyclohexanol[¹⁴C].

A mixture of the compound obtained above (2.8 mmol), (S)-aminotetrahydrofuran hydrochloride (346 mg, 2.8 mmol) and DIEA (1.0 mL, 2.1 mmol) in DCM (6 mL) was stirred at −70° C. (CO₂/i-PrOH) under an atmosphere of nitrogen. After minutes the cooling bath was removed and the mixture was stirred overnight at room temperature. TLC analysis of the crude reaction mixture ([EtOAc/hexanes (3:7)] Rf=0.56) showed the reaction to be complete. To the brown solution was then added DCM (2 mL), trans-4-aminocyclohexanol (323 mg, 2.8 mmol) and DIEA (1.0 mL, 2.1 mmol), and the reaction flask was sealed and stirred at 45° C. overnight. After cooling to room temperature the contents of the reaction flask was added to a saturated NH₄Cl solution (25 mL) and water (10 mL). The product was extracted [2×DCM (10 mL)] from the aqueous solution, dried (Na₂SO₄) and the solvent was removed under reduced pressure. The crude product was purified by flash chromatography over silica gel [EtOAc/heptane (9:1), EtOAc] to provide 0.49 g (54%) of the target compound as a light yellow solid. TLC (EtOAc) Rf=6.27. HPLC conditions C, RT=13.63 min (CP: 95.9%, radiochemical purity (RCP): 98.1%).

4-[(S)-9-(Tetrahydrofuran-3-yl)-8-(2,4,6-trifluorophenylamino)-9H-purin-2-ylaminocyclohexanol[¹⁴C].

A mixture of the compound obtained above (0.493 g, 1.52 mmol), 10% palladium on carbon (0.09 g, 0.08 mmol) and hydrazine hydrate (55%) (0.5 mL, 8.8 mmol) in MeOH (15 mL) was stirred overnight at 45° C. under nitrogen. 10% Palladium on carbon (0.09 g, 0.08 mmol) and hydrazine hydrate (55%) (0.5 mL, 8.8 mmol) was then added and the mixture was stirred at 45° C. for an additional 2 h. The reaction mixture was then cooled to room temperature and filtered through a pad of Celite. The celite pad was rinsed with MeOH (20 mL) and the combined filtrates were evaporated to dryness. Anhydrous THF was added to the residue and evaporated to dryness under reduced pressure (3×5 mL). The resulting product was dissolved in anhydrous THF (6 mL), 2,4,6-trifluoroisothiocyanate (0.31 g, 1.65 mmol) was added and the mixture was stirred at room temperature under nitrogen. The reaction was monitored via TLC ([MeOH/DCM (1:9)]). After approximately 2 h the reaction was complete, EDC (0.26 g, 1.7 mmol) was added and the reaction mixture was stirred for an additional 22 h. The reaction mixture was evaporated to dryness under reduced pressure and the crude product was purified by flash chromatography over silica gel [MeOH/DCM (1:24), MeOH/DCM (1:19)] to provide 0.30 g (44%) of the target compound as a tan solid: TLC (MeOH/DCM (1:9)) Rf: 0.38. The material was subjected to a second purification using semi-preparative reverse phase HPLC, condition B, RT˜14.8 min. The product was dissolved in a mixture of MeOH, CH₃CN and water (2.5:0.5:0.25) prior to loading and provided 0.27 g (40%) of target compound as a tan solid. HPLC condition C, RT=21.47 min (CP: 99.9%, RCP: 99.9%). ¹H NMR (300 MHz, CDCl₃) δ (ppm) 8.27 (s, 1H), 7.90 (s, 1H), 6.79 (t, J=8.0 Hz, 2H), 5.58-5.50 (m, 1H), 4.68 (d, J=8.2 Hz, 1H), 4.50-4.42 (m, 1H), 4.40 (d, J=11.0 Hz, 1H), 3.97-3.62 (comp, 3H), 2.77-2.62 (m, 1H), 2.35-1.98 (comp, 5H), 1.70-1.20 (comp, 5H).

Specific Activity.

Two samples of the compound obtained above (4.05 mg, and 4.71 mg) were weighed into individual vials and quantitatively transferred to 25 mL volumetric flasks with MeOH. Ten 100 μL aliquots were assayed for each sample using the Bio Safe II™ liquid scintillation cocktail. The average of decays per minute (dpm) value for the two samples was 4253467±32622 and 4728279±30787, resulting in a specific activity of 119.3 μCi/mg (53.8 mCi/mmol, 1990.6 MBq/mmol) and 114.1 μCi/mg (51.4 mCi/mmol, 1901.8 MBq/mmol). The average of the two calculations gives a specific activity of 116.7 μCi/mg (52.6 mCi/mmol, 1946.2 MBq/mmol).

### 5.2 Example 2

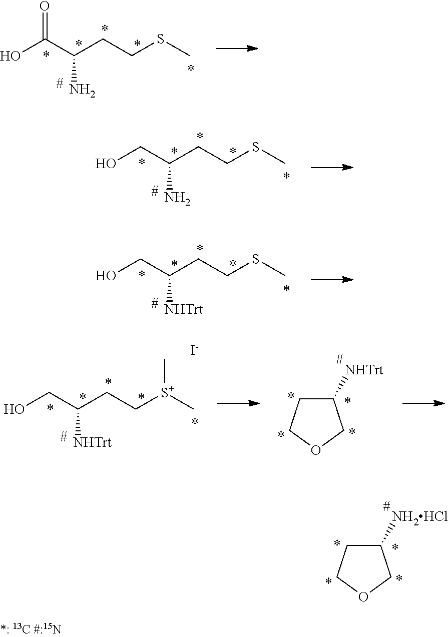

¹³C₅,¹⁵N-L-methioninol.

A 250 mL 3-neck round bottomed flask was charged with ¹³C₅,¹⁵N-L-Methionine (4.6 g, 29.65 mmol) and NaBH₄ (2.7 g, 71.46 mmol). Dry THF (106 mL) was added and the mixture was cooled to 0-5° C. (ice-MeOH bath) under N₂. A solution of I₂ (7.53 g, 29.65 mmol) in THF (50 mL) was added maintaining the temperature <2° C. The reaction mixture was allowed to warm to room temperature (20-25° C.) and then heated to 63-67° C. for 24 h. The mixture was cooled to 0-5° C. and MeOH (27.6 mL) was charged. The mixture was warmed to room temperature and stirred for 0.5 h followed by the addition of KOH (20% aq) (64.4 mL). After stirring at room temperature for 4 h, the mixture was charged to a separating funnel and the product was extracted with DCM (3×150 mL). The DCM layer was separated and washed with brine (1×30 mL), dried (Na₂SO₄) and concentrated giving ¹³C₅,¹⁵N-L-methioninol (3.57 g, 85%) as a colorless oil. ¹H NMR (300 MHz, D₂O) δ (ppm) 3.80-3.47 (m, 1H), 3.32-2.99 (m, 2H), 2.88-2.53 (m, 2H), 2.42-2.17 (m, 2H), 1.94-1.60 (m, 2H), 1.56-1.17 (m, 1H)

N-Trt-¹³C₅,¹⁵N-Methioninol.

A 250 mL 3-neck round bottomed flask was charged with the compound obtained above (3.54 g, 25.07 mmol)) and DCM (35.4 mL). The mixture was cooled to 0-5° C. (ice-MeOH bath) under N₂ and TEA (5.07 g, 50.14 mmol) in DCM (5 vol) was added followed by a solution of Trt-Cl (6.99 g, 25.07 mmol) in DCM (5 vol). The reaction mixture was allowed to warm to room temperature (20-25° C.) and then stirred for 20 h. The mixture was transferred to a separating funnel, washed with 10% citric acid (3×100 mL), water (1×100 mL) and brine (1×50 mL), then dried (Na₂SO₄) and concentrated to give N-Trt-¹³C₅,¹⁵N-methioninol (9.61 g, 100%) as a viscous oil. ¹H NMR (300 MHz, DMSO-d₆) δ (ppm) 7.50 (d, J=7.6 Hz, 5H), 7.38-7.06 (m, 100H), 4.38 (br. s., 1H), 3.60 (t, J=6.5 Hz, 3H), 3.31-3.19 (m, 1H), 2.96 (br.s., 1H), 2.85-2.55 (m, 1H), 2.44-2.04 (m, 2H), 2.02-1.85 (m, 1H), 1.82-1.51 (m, 2H). LC-MS ES⁺ (M+1) 142 (parent), 243 (+Trt).

N-Trt-¹³C₅,¹⁵N-Methioninol sulfenyl iodide.

A 250 mL 3-neck round bottomed flask was charged with the compound obtained above (9.61 g, 25.07 mmol) and MeCN (67 mL) under N₂. MeI (14.23 g, 100.3 mmol) was added, followed by MeCN (2 vol) and the mixture was heated to 40° C. for 18 h. The mixture was cooled to 0-10° C. for 1 h. The product was collected by filtration, washed with MeCN (2 vol) then dried in vacuo for 6 h to give N-Trt-¹³C₅,¹⁵N-methioninol sulfenyl iodide (9.41 g, 71%) as an off-white solid. ¹H NMR (300 MHz, DMSO-d₆) (ppm) 7.67-7.10 (m, 15H), 4.56 (br. s., 1H), 3.61-3.37 (m, 1H), 3.26-2.55 (m, 8H), 2.38 (br. s., 1H), 1.74 (br. s., 2H), 1.30 (br. s., 2H).

N-Trt-¹³C₄,¹⁵N-3-(s)-Aminotetrahydrofuran.

A 250 mL 3-neck round bottomed flask was charged with the compound obtained above (10.15 g, 19.32 mmol) and THF (132 mL), then cooled to −15 to −10° C. under N₂. t-BuOK (7.16 g, 63.76 mmol) was charged, maintaining the temperature <−5° C. After the addition was complete, the mixture was warmed to room temperature over 1.5 h. The mixture was cooled to −15 to −10° C. and quenched with glacial AcOH (3.83 g, 63.76 mmol), maintaining the temperature <−5° C. The mixture was allowed to warm to room temperature and then diluted with H₂O (4 vol) and EtOAc (15 vol). The organic layer was separated, washed with 5% aq. Na₂S₂O₃ (3×100 mL), water (4×100 mL) and brine (1×50 mL). The organic layer was dried (Na₂SO₄) and concentrated to a slurry. DCM (3×50 mL) was charged to afford a solid, which was collected by filtration, dried in vacuo at room temperature for 18 h to give N-Trt-¹³C₄,¹⁵N-3-(s)-aminotetrahydrofuran (6.41 g, 99.2%) as an off-white solid. HPLC: Hypersil DBS C8, 4.6×250 mm, 5, 35° C., gradient (1/99 to 85/15: MeCN/10 mmol aq. KH₂PO₄), 1.0 mL/min, 20 min: RT 17.20 min, 87.32% AP at 240 nm. ¹H NMR (300 MHz, DMSO-d₆) δ (ppm) 7.68-7.04 (m, 15H), 3.90-3.52 (m, 1H), 3.28-3.01 (m, 2H), 2.91-2.56 (m, 2H), 1.65 (br. s., 1H), 1.32-1.04 (m, 1H).

¹³C₄,¹⁵N-3-(s)-Aminotetrahydrofuran.

A 250 mL 3-neck round bottomed flask was charged with the compound obtained above (6.25 g, 18.7 mmol) and iPrOH (55 mL) under N₂. The mixture was heated to 75-77° C. for 1.5 h. The mixture was cooled to room temperature and concentrated to remove ˜3 vol solvent. MTBE (10 vol) was charged and the mixture stirred at room temperature for 4 h. The product was collected by filtration, washed with cold (5-10° C.) MTBE then dried in vacuo at room temperature for 18 h to give ¹³C₄,¹⁵N-3-(s)-aminotetrahydrofuran (1.74 g, 73%) as an off-white solid. ¹H NMR (300 MHz, DMSO-d₆) δ (ppm) 8.69-8.00 (m, 3H), 4.22-3.82 (m, 3H), 3.72-3.39 (m, 2H), 2.47-2.03 (m, 1H), 2.03-1.57 (m, 1H).

Other isotopically enriched aminotetrahydrofuran derivatives (enriched with deuterium, ¹⁵N, ¹⁴C, ¹³C, ¹⁸O and combinations thereof) can similarly be prepared from the corresponding isotopically enriched methioninol starting materials that are either commercially available or can be readily synthesized by one of ordinary skilled in the art. Incorporation of the isotopically enriched aminotetrahydrofuran derivatives is achieved as exemplified below.

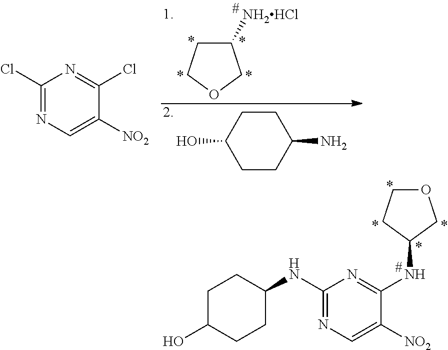

¹³C₄,¹⁵N-4-(5-Nitro-4-((S)-tetrahydrofuran-3-ylamino)pyrimidin-2-ylamino)cyclohexanol. A 250 mL 3-neck round bottomed flask was charged with 2,4-dichloro-5-nitropyrimidine (2.54 g, 13.07 mmol) and DCM (25 mL) and cooled to −30 to −20° C. under N₂. ¹³C₅, ¹⁵N-3-(s)-aminotetrahydrofuran was charged followed by DIEA (3.55 g, 27.45 mmol) maintaining the temperature <−15° C. The reaction mixture was allowed to warm to room temperature (20-25° C.) and stirred for 4 h. trans-4-Aminocyclohexanol (1.51 g, 13.07 mmol) was charged together with DIEA (2.03 g, 15.7 mmol) in one portion. The mixture was stirred at room temperature for 0.5-1 h and then heated to 35-38° C. for 18 h. The DCM was distilled off until ˜6 vol remained and MeCN (3 vol) was charged. The mixture was cooled to 0-5° C. and water (5 vol) was charged. The mixture was allowed to stand for 1-2 h and then the product was collected by filtration, washed with water (2×5 vol), MTBE (2×5 vol) and dried in vacuo at 35-40° C. for 15 h, giving the target compound (3.4 g, 79%) as a yellow solid. HPLC: DBS C8, 4.6×250 mm, 5μ, 35° C., gradient (1/99 to 85/15: MeCN/10 mmol aq. KH₂PO₄), 1.0 mL/min, 20 min: RT 10.06 min, 96.73% AP at 240 nm. LC-MS ES⁺ (M+1) 329.

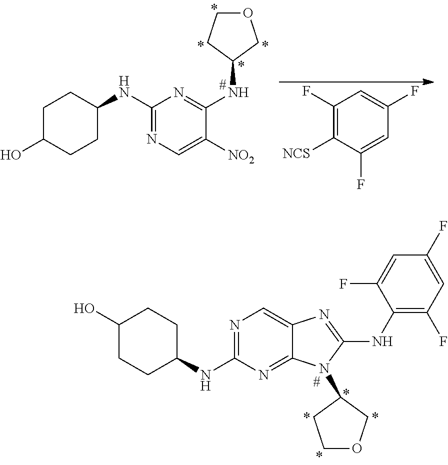

¹³C₄,¹⁵N-4-[(S)-9-(Tetrahydrofuran-3-yl)-8-(2,4,6-trifluorophenylamino)-9H-purin-2-ylaminocyclohexanol.

A Parr hydrogenation container was charged with the compound obtained above (3.26 g, 9.92 mmol), 10% Pd/C (50% wet) (0.326 g, 10 wt %) and MeOH (65 mL). The mixture was hydrogenated at 40-50 psi, 30-35° C. for 3 h. The catalyst was removed by filtration through celite, which was then washed with warm (30-35° C.) MeOH (12 vol). The filtrate was concentrated to an oil and then redissolved in THF (65 mL). The product was carried through to the next stage as a solution in THF (100% yield, 2.96 g was assumed). HPLC: Hypersil DBS C8, 4.6×250 mm, 5p, 35° C., gradient (1/99 to 85/15: MeCN/10 mmol aq. KH₂PO₄), 1.0 mL/min, 20 min: RT 6.84 min, 98.73% AP at 240 nm. LC-MS ES⁺ (M+1) 299.

A 250 mL 3-neck round bottomed flask was charged with the compound obtained above (2.96 g, 9.92 mmol) and THF (45 mL) under N₂. 1,3,5-Trifluoro-2-thiocyanatobenzene (1.88 g, 9.92 mmol) was charged in one portion maintaining 18-20° C. using a cold water bath. After 18 h at room temperature (20-25° C.), EDC was charged and the mixture heated to 58-60° C. After 3-4 h, AcOH and water were charged and stirring continued at 60-62° C. for 1.5 h. The mixture was cooled to room temperature and EtOAc was Added. The reaction mixture was charged to a separating funnel and the organic layer was separated and washed with water (2×2 vol), aq. Na₂CO₃ (3×2 vol) then water (2×2 vol). The organic layer was concentrated, redissolved in EtOH (8 vol) and heated to 80-83° C. Water (25 vol) was charged maintaining the temperature >75° C. and the mixture was cooled to room temperature over 3 h. The product was collected by filtration, washed with water (2×5 vol) and dried in vacuo for 6 h, giving crude target compound (3.2 g, 71%) as an off-white solid. HPLC: Hypersil DBS C8, 4.6×250 mm, 5μ, 35° C., gradient (1/99 to 85/15: MeCN/10 mmol aq. KH₂PO₄), 1.0 mL/min, 20 min: RT 9.93 min, 98.13% AP at 240 nm. LC-MS ES⁺ (M+1) 454.

A 250 mL 3-neck round bottomed flask was charged with crude compound from above (3.0 g, 6.61 mmol) and EtOH (25.5 mL) and heated to 78-83° C. The solution was polish filtered, the filtrate charged to a clean 250 mL 3-neck round bottomed flask and reheated to 78-83° C. Water was charged maintaining the temperature >75° C. Once the addition was complete, the mixture was cooled to room temperature (21-23° C.) over 2.5 h. The product was collected by filtration, washed with water (2×5 vol) and dried at 35-40° C. in vacuo for 49 h, giving the target compound (2.63 g, 88% recovery) as an off-white solid. HPLC: Hypersil DBS C8, 4.6×250 mm, 5μ, 35° C., gradient (1/99 to 85/15: MeCN/10 mmol aq. KH₂PO₄), 1.0 mL/min, 20 min: RT 9.93 min, 99.70% AP at 240 nm. ¹H-NMR (300 MHz, METHANOL-d₄) δ (ppm) 8.03 (br s, 1H), 7.00 (br s, 2H), 5.54 (br s, 0.5H), 5.05 (br s, 0.5H), 4.66-4.55 (br d, 1H), 4.35-4.05 (m, 2H), 3.85-3.56 (m, 3H), 2.82 (br s, 1H), 2.38 (br s, 1H), 2.23-1.93 (m, 4H), 1.51-1.36 (m, 4H). ¹³C-NMR (75 MHz, DMSO-d₆) δ (ppm) 157.36-156.97 (t), 154.04-153.83 (m), 153.57-153.30 (d), 152.01-149.59 (d), 147.90-147.60 (d), 143.57 (s), 133.60 (s), 125.15-125.07 (d), 144.63 (s), 101.35-100.16 (m), 69.48-67.36 (m), 53.52-50.86 (m), 34.31 (s), 30.26-27.84 (m). LC-MS ES⁺ (M+1) 454. CHN-Analysis, calcd for C₁₇¹³C₄H₂₃F₃N₅¹⁵NO₂: C, 55.62%; H, 5.11%; N, 18.54%. Found: C, 55.81%; H, 5.23%; N, 18.54%. m.p. 226° C.

### 5.3 Example 3

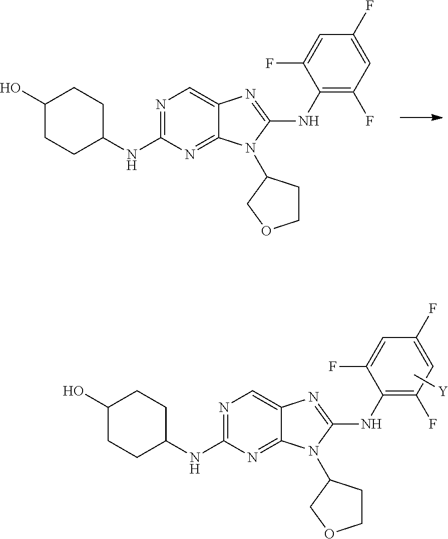

The isoindoline portion of Compound 1 is deuterated by subjecting Compound 1 to conditions suitable for aromatic deuteration, which are known in the art, including for example, those disclosed in the following references, each of which are incorporated herein by reference in their entireties: U.S. Publication No. 2007/0255076; March, J. “Advanced Organic Chemistry, Reactions, Mechanisms, and Structure,” Fourth Ed., Wiley, New York, 1992; Larsen et al., J. Org. Chem., 43(18), 3602, 1978; Blake et al., J. Chem. Soc., Chem. Commun., 930, 1975; and references cited therein. For example, Compound 1 is treated with D₂O over 5% Pt/C under hydrogen gas to provide an isotopologue of compound 1, as depicted in the scheme above.

### 5.4 Example 4

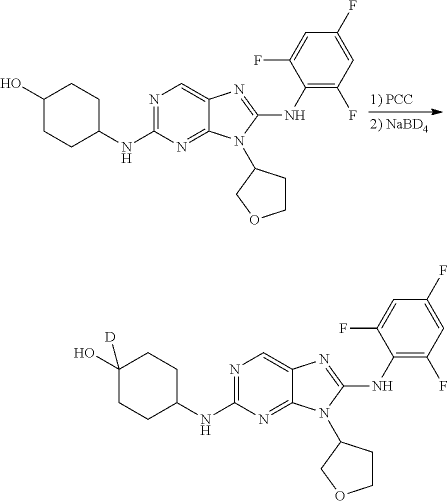 [SMILES: OC1CCC(Nc2ncc3nc(Nc4c(F)cc(F)cc4F)n(C4CCOC4)c3n2)CC1 ; [2H]C1(O)CCC(Nc2ncc3nc(Nc4c(F)cc(F)cc4F)n(C4CCOC4)c3n2)CC1]

The aminocyclohexanol portion of Compound 1 can be deuterated by oxidation of the hydroxyl moiety (with for example, pyridinium chlorochromate) followed by reduction in the presence of NaBD₄, to afford the isotopologue of Compound 1 as shown above.

### 5.5 Example 5

The following isotopically enriched starting materials can be used in the methods described herein to afford additional isotopologues of Compound 1.

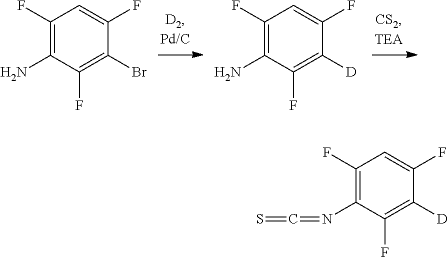 [SMILES: Nc1c(F)cc(F)c(Br)c1F ; [2H]c1c(F)cc(F)c(N)c1F ; [2H]c1c(F)cc(F)c(N=C=S)c1F]

Deuterated trifluoroaniline starting materials for deuterium enriched isotopologues of Compound 1 can be prepared by methods known in the art. For example, as shown in the scheme above, 1,3,5-trifluoro-2-isothiocyanatobenzene-²H can be prepared from commercially available 3-bromo-2,4,6-trifluoroaniline by hydrogenation with D₂ in the presence of Pd/C, followed by conversion to the isothiocyanate derivative by treatment with carbon disulfide in the presence of a base such as TEA (J. Li et. al. Bioorganic & Medicinal Chemistry, 17(8): 3177-3188 (2009)).

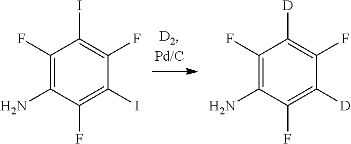 [SMILES: Nc1c(F)c(I)c(F)c(I)c1F ; [2H]c1c(F)c([2H])c(F)c(N)c1F]

Similarly, 2,4,6-trifluoro-3,5-diiodoaniline (W. Sander, et. al. J. Org. Chem. 72(3): 715-724 (2007)), can be bis-deuterated by treatment with D₂ in the presence of Pd/C. As above, conversion to the isothiocyanate provides the precursor for the synthesis of the bis-deuterated isotopologue of Compound 1.

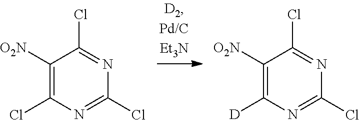 [SMILES: O=[N+]([O-])c1c(Cl)nc(Cl)nc1Cl ; [2H]c1nc(Cl)nc(Cl)c1[N+](=O)[O-]]

Deuterated halogenated nitropyrimidine starting materials can also be prepared using methods similar to those known in the art (e.g. Baasner, B. et al, J. Fluorine Chem. 45(3): 417-30 (1989)). For example, as shown above, 2,4,6-trichloro-5-nitropyrimidine can be treated with D₂ in the presence of Pd/C to provide the deuterated starting material, to be used in the synthesis methods described herein.

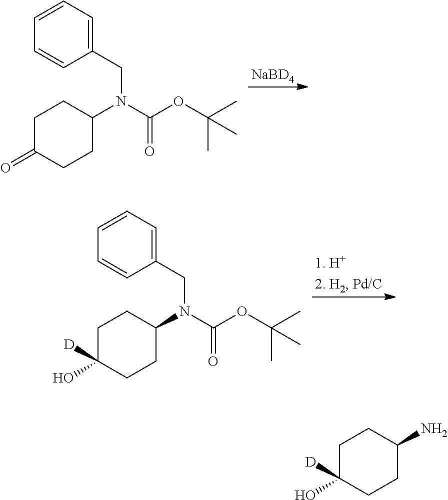 [SMILES: CC(C)(C)OC(=O)N(Cc1ccccc1)C1CCC(=O)CC1 ; "NaBD4" ; [2H][C@]1(O)CC[C@H](N(Cc2ccccc2)C(=O)OC(C)(C)C)CC1 ; [2H][C@]1(O)CC[C@H](N)CC1]

Deuterated aminocychohexyl starting materials can be prepared using methods similar to those known in the art (e.g. Quirante, J. et al, J. Org. Chem. 67(7): 2323-2328 (2002)). For example, as shown above, tert-butyl benzyl(4-oxocyclohexyl)-carbamate can be reduced with NaBD₄ to provide deuterated tert-butyl benzyl((1r,4r)-4-hydroxy-cyclohexyl)carbamate which, after deprotection of the tert-butyloxycarbonyl and benzyl protecting groups, can be incorporated by the synthesis methods described herein.

### 5.6 Determination of Isotopic Enrichment

Isotopic enrichment may be confirmed may be confirmed and quantified by mass spectrometry and/or NMR, including, for example, proton-NMR; carbon-13NMR; or nitrogen-15NMR.

Isotopic enrichment may also be confirmed by single-crystal neutron diffraction. For example, the isotopic ratio at a particular hydrogen/deuterium position in a deuterated Compound 1 can be determined using single-crystal neutron diffraction. Neutron diffraction is advantageous because neutrons are scattered by the nucleus of an atom, therefore allowing for discrimination between isotopes, such as hydrogen and deuterium, which differ in the number of neutrons in the nucleus.

A single crystal of suitable size and quality comprising the deuterated Compound 1 is grown using standard methods of crystal growth. For single-crystal neutron diffraction experiments, crystals of several cubic millimeters are generally required for suitable data collection. A minimum size for a single crystal is typically about 1 cubic millimeter. Suitable single crystals are obtained by dissolving the deuterated Compound 1 in a solvent with appreciable solubility, then slowly evaporating or cooling the solution to yield crystals of suitable size and quality. Alternatively, suitable single crystals are obtained by dissolving the deuterated Compound 1 in a solvent with appreciable solubility, then slowly diffusing into the solution of antisolvent (i.e., a solvent in which the deuterated Compound 1 is not appreciably soluble) to yield crystals of suitable size and quality. These and other suitable methods of crystal growth are known in the art and are described, e.g., in George H. Stout & Lyle H. Jensen, X-Ray Structure Determination: A Practical Guide 74-92 (John Wiley & Sons, Inc. 2nd ed. 1989) (the entirety of which is incorporated herein).

After isolating a suitable single crystal comprising the deuterated Compound 1, the crystal is mounted in a neutron beam, neutron diffraction data is collected, and the crystal structure is solved and refined. Different neutron sources can be used, including steady-state sources and pulsed spallation sources. Examples of steady-state sources include the Grenoble ILL High Flux Reactor (Grenoble, France) and the Oak Ridge High Flux Isotope Reactor (Oak Ridge, Tenn.). Examples of pulsed spallation sources include ISIS, the spallation neutron source at Rutherford Appleton Laboratory (Oxfordshire, UK); the Intense Pulsed Neutron Source (IPNS) at Argonne National Laboratory (Argonne, Ill.), the Los Alamos Neutron Science Center (LANSCE) at Los Alamos National Laboratory (Los Alamos, N. Mex.), and the Neutron Science Laboratory (KENS) at KEK (Tsukuba, Ibaraki, Japan).

For a steady-state neutron source, four-circle diffractometer techniques are used with a monochromatic beam and a single detector, rotating the crystal and detector to measure each reflection sequentially. Diffractometer control software and step-scanning methods for intensity extraction can be adopted from routine four-circle X-ray diffractometry methods. One or more area detectors, including area detector arrays, may alternatively be used to increase the region of reciprocal space accessed in a single measurement. A broad band (white) beam used with an area detector allows for Laue or quasi-Laue diffraction with a stationary crystal and detector.

For a pulse source with a white neutron beam, time-of-flight Laue diffraction techniques are used, which allow for the determination of the velocity, energy, and wavelength of each neutron detected. This approach combines wavelength sorting with large area position-sensitive detectors, and allows for fixed scattering geometries (i.e., a stationary crystal and detector). Pulse source data collected in this fashion allows for rapid collection of data sets and good accuracy and precision in standard structural refinements. Additional details regarding steady-state and pulse source neutron diffraction experiments are well known in the art. See, e.g., Chick C. Wilson, Neutron Single Crystal Diffraction, 220 Z. Kristallogr. 385-98 (2005) (incorporated by reference herein in its entirety).

Crystal structure data, including particular isotopic ratios, are obtained from neutron diffraction data following routine structure solution and refinement processes. Structure solution is carried out using one of several methods, including direct methods and Patterson methods. For convenience, atomic coordinates from prior single crystal X-ray diffraction experiments may be used as a starting point for structure refinement using neutron diffraction data; this approach permits additional refinement of atomic positions, including hydrogen and deuterium positions. Refinement is conducted using full-matrix least-squares methods to achieve optimal agreement between the observed diffraction intensities and those calculated from the structural model. Ideally, full anisotropic refinement is carried out on all atoms, including the H/D atomic positions of interest. Data collection, structure solution and structure refinement methods, both for X-ray and neutron diffraction data, are well known in the art. See, e.g., Chick C. Wilson, Single Crystal Neutron Diffraction from Molecular Materials (World Scientific Publishing Co. 2000); George H. Stout & Lyle H. Jensen, X-Ray Structure Determination: A Practical Guide (John Wiley & Sons, Inc. 2nd ed. 1989) (both of which are incorporated herein in their entireties).

The isotopic ratio for a particular position on a deuterated Compound 1 is calculated by examining the neutron scattering cross sections for the H/D atomic position of interest. The scattering cross section is obtained as part of the refinement process discussed above. An example of determining the isotopic ratio for a partially deuterated compound is provided by G. A. Jeffrey et al., Neutron Diffraction Refinement of Partially Deuterated β-D-Arabinopyranose and α-L-Xylopyranose at 123 K, B36 Acta Crystallographica 373-77 (1980) (incorporated by reference herein in its entirety). Jeffrey et al. used single-crystal neutron diffraction to determine the percentage deuterium substitution for hydroxyl groups on two sugar compounds of interest. Employing the methods discussed by Jeffrey et al., one may similarly ascertain the isotopic ratio for a particular H/D position on a deuterated Compound 1.

All of the cited references are incorporated herein by reference in their entirety.

What is claimed is:

1. A compound, wherein the compound is an isotopologue of Compound 1 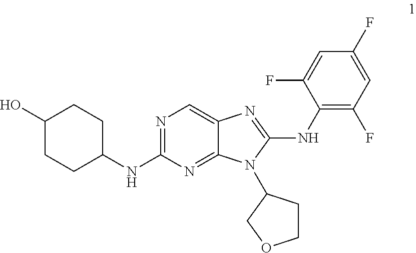 [SMILES: OC1CCC(Nc2ncc3nc(Nc4c(F)cc(F)cc4F)n(C4CCOC4)c3n2)CC1]
or a pharmaceutically acceptable salt or solvate thereof.

2. The compound of claim 1, wherein the isotopologue is an isotopologue of

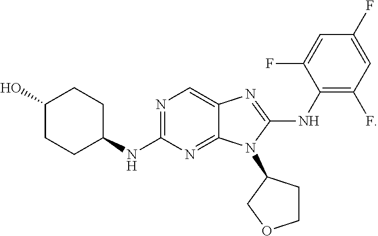 [SMILES: O[C@H]1CC[C@H](Nc2ncc3nc(Nc4c(F)cc(F)cc4F)n([C@H]4CCOC4)c3n2)CC1]

3. The compound of claim 1, wherein the isotopologues are deuterium, carbon-13, or nitrogen-15 enriched, or combinations thereof.

4. The compound of claim 1, wherein the isotopologue is a compound of formula: 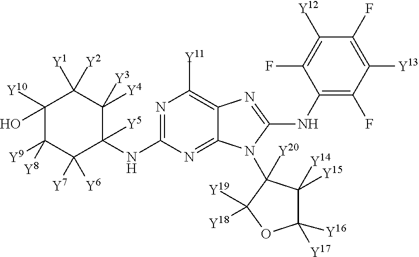
wherein one or more of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷, Y¹⁸, Y¹⁹, and Y²⁰ is a hydrogen that is isotopically enriched with deuterium, and the others of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷, Y¹⁸, Y¹⁹, and Y²⁰ are non-enriched hydrogen atoms.

5. The compound of claim 4, wherein one of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷, Y¹⁸, Y¹⁹, and Y²⁰ is isotopically enriched with deuterium, and the others are non-enriched hydrogens.

6. The compound of claim 4, wherein two of Y¹, Y², Y³, Y⁴, Y⁵, Y⁶, Y⁷, Y⁸, Y⁹, Y¹⁰, Y¹¹, Y¹², Y¹³, Y¹⁴, Y¹⁵, Y¹⁶, Y¹⁷, Y¹⁸, Y¹⁹, and ²⁰ are isotopically enriched with deuterium, and the others are non-enriched hydrogens.

7. The compound of claim 4, wherein the compound is selected from

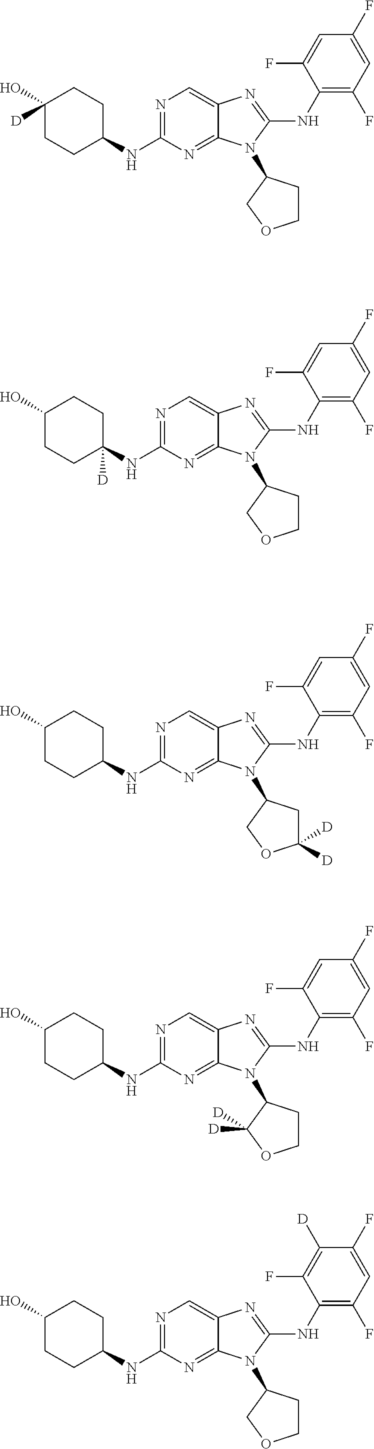

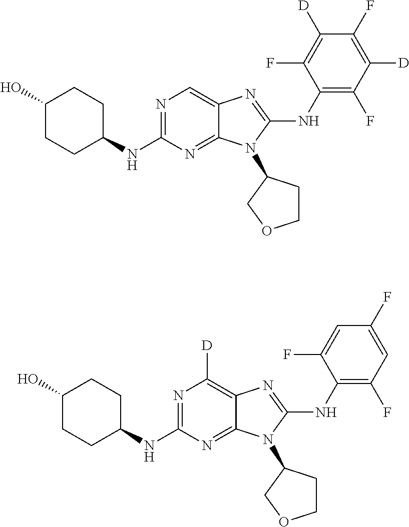 [SMILES: [2H]c1c(F)c([2H])c(F)c(Nc2nc3cnc(N[C@H]4CC[C@H](O)CC4)nc3n2[C@H]2CCOC2)c1F ; [2H]c1nc(N[C@H]2CC[C@H](O)CC2)nc2c1nc(Nc1c(F)cc(F)cc1F)n2[C@H]1CCOC1]

8. The compound of claim 1, wherein the isotopologue is a compound of formula: 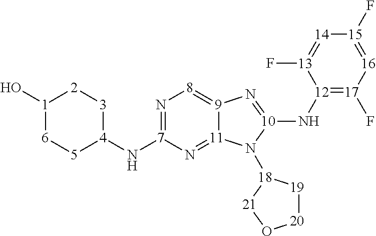
wherein 1, 2, 3, 4, 5, 6, 7, 8, 9, 10, 11, 12, 13, 14, 15, 16, 17, 18, 19, 20 and 21 are carbon atoms; and at one or more of 1, 2, 3, 4, 5, 6, 7, 8, 9, 10, 11, 12, 13, 14, 15, 16, 17, 18, 19, 20 or 21 is isotopically enriched with carbon-13.

9. The compound of claim 8, wherein the compound is

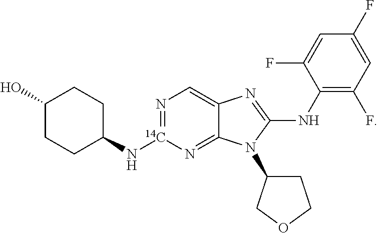 [SMILES: O[C@H]1CC[C@H](N[14c]2ncc3nc(Nc4c(F)cc(F)cc4F)n([C@H]4CCOC4)c3n2)CC1]

10. The compound of claim 1, wherein the isotopologue is a compound of formula: 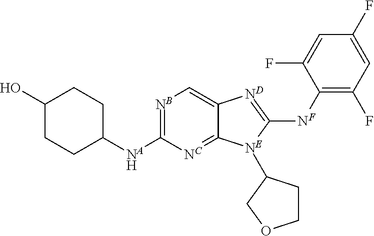
wherein N^{A}, N^{B}, N^{C}, N^{D}, N^{E} and N^{F} are nitrogen atoms; and one or more of N^{A}, N^{B}, N^{C}, N^{D}, N^{E} or N^{F} is isotopically enriched with nitrogen-15.

11. The compound of claim 1, wherein the compound is

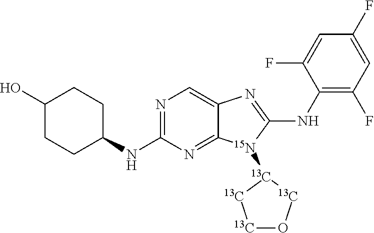

12. A pharmaceutical composition comprising a compound of claim 1, or a pharmaceutically acceptable salt or solvate thereof.

13. A method of treating, managing or preventing a disease or disorder comprising administering to a patient a compound of claim 1, or a pharmaceutically acceptable salt or solvate thereof, wherein the disease or disorder is cancer, cardiovascular disease, inflammatory disease, autoimmune disease or a metabolic disorder.

14. The method of claim 13, further comprising administering a second active agent.
